(19) United States
(12) Patent Application Publication
(10) Pub. No.: US 2026/0209189 A1
(43) Pub. Date: Jul. 23, 2026

(54) ISOXAZOLINE-SUBSTITUTED BENZAMIDE COMPOUND AND APPLICATION THEREOF

(71) Applicant: QINGDAO KINGAGROOT CHEMICAL COMPOUND CO., LTD., Qingdao, Shandong (CN)

(72) Inventors: Lei LIAN, Qingdao, Shandong (CN); Rongbao HUA, Qingdao, Shandong (CN); Xuegang PENG, Qingdao, Shandong (CN)

(21) Appl. No.: 19/116,599

(22) PCT Filed: Jan. 4, 2023
(86) PCT No.: PCT/CN2023/070433

§ 371 Date: Mar. 28, 2025

(30) Foreign Application Priority Data
Oct. 1, 2022 (CN) 202211217148.1
Oct. 29, 2022 (CN) 202211339356.9

(51) Int. Cl. C07D 261/04; A01N 43/80; A01P 7/04; C07D 413/04

(52) U.S. Cl. CPC C07D 261/04; A01N 43/80; C07D 413/04; A01P 7/04

(57) ABSTRACT

The present invention provides an isoxazoline-substituted benzamide compound, a stereoisomer and an agriculturally or veterinarily acceptable salt thereof. The compound is as shown in Formula I:

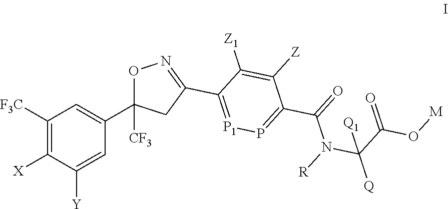

wherein, X represents hydrogen, halogen, alkoxy or alkylthio; Y represents halogen or haloalkyl; Z and Z₁ each independently represent hydrogen, halogen, etc.; P and P₁ each independently represent CH or N; Q and Q₁ each independently represent alkyl, etc.; M represents hydrogen, alkyl, etc.; R represents hydrogen, alkyl, etc. The compound has excellent control effects on pest insects such as fall armyworm (Spodoptera frugiperda), beet armyworm (Spodoptera exigua), groundnut aphid (Aphis craccivora), two-spotted spider mite (Tetranychus urticae), striped rice stem borer (Chilo suppressalis), cotton aphid (Aphis gossypii), etc.

### TECHNICAL FIELD

The present invention belongs to the technical field of pesticides, and in particular relates to an isoxazoline-substituted benzamide compound and an application thereof.

### BACKGROUND OF THE INVENTION

In recent years, owing to the long-term use of pest control agents such as insecticides or fungicides, diseases and pests have acquired drug resistance and become difficult to control by currently used insecticides or fungicides. In addition, part of the known pest control agents is highly toxic, or some of which damage the ecosystem by their long-term residue. In this case, although a large number of insecticides are known, for example, WO2015/128358 discloses azoline compounds and their application as insecticides, it is still necessary to develop new pest control agents with low toxicity and low residue.

### BRIEF SUMMARY OF THE INVENTION

In order to solve the above-mentioned problems existing in the prior art, the present invention provides an isoxazoline-substituted benzamide compound, a stereoisomer and an agriculturally or veterinarily acceptable salt thereof, and the compound has excellent control effects on pest insects such as fall armyworm (Spodoptera frugiperda), beet armyworm (Spodoptera exigua), groundnut aphid (Aphis craccivora), two-spotted spider mite (Tetranychus urticae), striped rice stem borer (Chilo suppressalis), cotton aphid (Aphis gossypii), etc.

### DETAILED DESCRIPTION OF THE INVENTION

The technical scheme adopted in the present invention is as follows:
An isoxazoline-substituted benzamide compound as shown in Formula I, a stereoisomer and an agriculturally or veterinarily acceptable salt thereof:

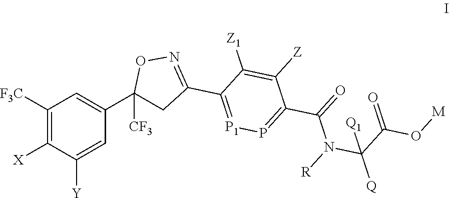

wherein, X represents hydrogen, halogen, alkoxy or alkylthio;
Y represents halogen or haloalkyl;
Z and Z₁ each independently represent hydrogen, halogen, alkyl, alkenyl, alkynyl, cycloalkyl, haloalkyl or cyano;
P and P₁ each independently represent CH or N;
Q and Q₁ each independently represent hydrogen, halogen, hydroxyl, cyano, amino, nitro, formyl, cyanoalkyl, hydroxyalkyl, alkyl, alkenyl, alkynyl, cycloalkyl, cycloalkylalkyl, alkoxy, alkylthio, alkylamino, haloalkoxy, haloalkylthio, alkylcarbonyl, alkoxycarbonyl, alkoxyalkyl, haloalkoxyalkyl, alkylaminoalkyl, aryl, heterocyclyl, arylalkyl or heterocyclylalkyl; wherein, the “alkyl”, “alkenyl” or “alkynyl” are each independently unsubstituted or substituted by at least one group of halogen, —OR₁, —SR₁, —(CO)OR₁, —(CO)N(R₁)₂, —(SO₂)R₁ or —N(R₁)₂; the “cycloalkyl”, “cycloalkylalkyl”, “aryl”, “heterocyclyl”, “arylalkyl” or “heterocyclylalkyl” are each independently unsubstituted or substituted by at least one group selected from oxo, halogen, cyano, nitro, alkyl, alkenyl, alkynyl, cycloalkyl, haloalkyl, haloalkenyl, haloalkynyl, halocycloalkyl, cycloalkyl substituted by alkyl, —OR₁, —SR₁, —(CO)OR₁, —(SO₂)R₁, —N(R₁)₂ or —O— alkyl-(CO)OR₁, or two adjacent carbon atoms on the ring form a fused ring with —OCH₂CH₂— or —OCH₂O— that is unsubstituted or substituted by halogen;
M represents hydrogen, alkyl, alkenyl, alkynyl, cycloalkyl, cycloalkenyl, heterocyclyl, aryl,

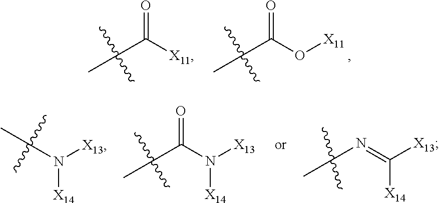

wherein, the “alkyl”, “alkenyl” or “alkynyl” are each independently unsubstituted or substituted by at least one group selected from halogen, cyano, nitro, cycloalkyl, trialkylsilyl, cycloalkenyl, heterocyclyl, aryl,

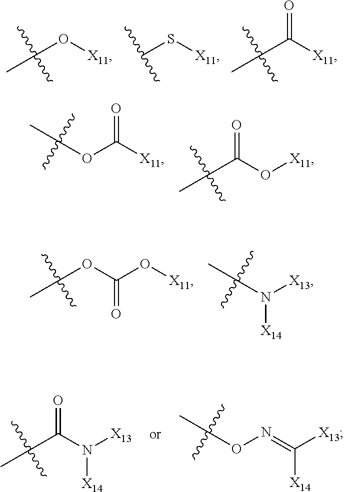

the “cycloalkyl”, “cycloalkenyl”, “heterocyclyl” or “aryl” are each independently unsubstituted or substituted by at least one group selected from oxo, halogen, cyano, nitro, alkyl, alkenyl, alkynyl, cycloalkyl, haloalkyl, haloalkenyl, haloalkynyl, halocycloalkyl, cycloalkyl substituted by alkyl, —OR₁, —SR₁, —(CO)OR₁, —(SO₂)R₁, —N(R₁)₂ or —O-alkyl-(CO)OR₁, or two adjacent carbon atoms on the ring form a fused ring with —OCH₂CH₂— or —OCH₂O— that is unsubstituted or substituted by halogen;
R represents hydrogen, alkyl, alkenyl, alkynyl, cycloalkyl, cycloalkenyl, heterocyclyl, aryl,

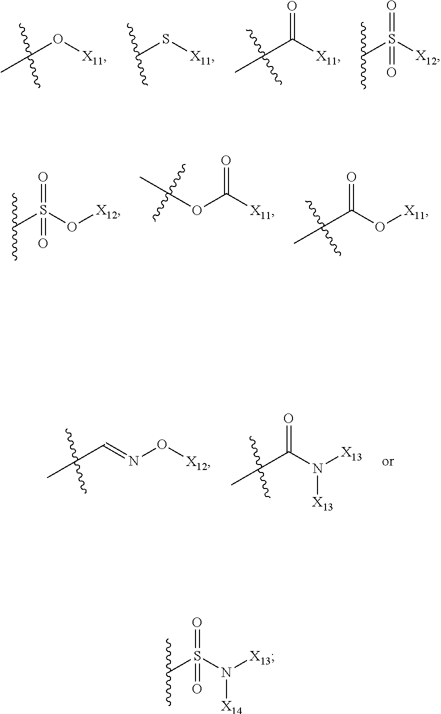

wherein, the “alkyl”, “alkenyl” or “alkynyl” are each independently unsubstituted or substituted by at least one group selected from halogen, cyano, nitro, cycloalkyl, trialkylsilyl, cycloalkenyl, heterocyclyl, aryl,

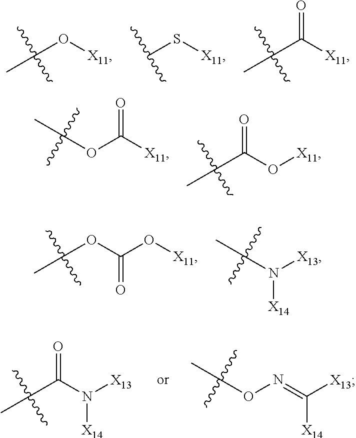

the “cycloalkyl”, “cycloalkenyl”, “heterocyclyl” or “aryl” are each independently unsubstituted or substituted by at least one group selected from oxo, halogen, cyano, nitro, alkyl, alkenyl, alkynyl, cycloalkyl, haloalkyl, haloalkenyl, haloalkynyl, halocycloalkyl, cycloalkyl substituted by alkyl, —OR₁, —SR₁, —(CO)OR₁, —(SO₂)R₁, —N(R₁)₂ or —O— alkyl-(CO)OR₁, or two adjacent carbon atoms on the ring form a fused ring with —OCH₂CH₂— or —OCH₂O— that is unsubstituted or substituted by halogen;
X₁₁ each independently represents hydrogen, alkyl, alkenyl, alkynyl, cycloalkyl, cycloalkylalkyl, cycloalkenyl, cycloalkenylalkyl, heterocyclyl, heterocyclylalkyl, aryl or arylalkyl; wherein, the “alkyl”, “alkenyl” or “alkynyl” are each independently unsubstituted or substituted by at least one group of halogen, —OR₁, —SR₁, —(CO)OR₁, —(SO₂)R₁ or —N(R₁)₂; the “cycloalkyl”, “cycloalkylalkyl”, “cycloalkenyl”, “cycloalkenylalkyl”, “heterocyclyl”, “heterocyclylalkyl”, “aryl” or “arylalkyl” are each independently unsubstituted or substituted by at least one group selected from oxo, halogen, cyano, nitro, alkyl, alkenyl, alkynyl, cycloalkyl, haloalkyl, haloalkenyl, haloalkynyl, halocycloalkyl, cycloalkyl substituted by alkyl, —OR₁, —SR₁, —(CO)OR₁, —(SO₂)R₁, —N(R₁)₂ or —O-alkyl-(CO)OR₁, or two adjacent carbon atoms on the ring form a fused ring with —OCH₂CH₂— or —OCH₂O— that is unsubstituted or substituted by halogen;
X₁₂ each independently represents alkyl, alkenyl, alkynyl, cycloalkyl, cycloalkylalkyl, cycloalkenyl, cycloalkenylalkyl, heterocyclyl, heterocyclylalkyl, aryl or arylalkyl; wherein, the “alkyl”, “alkenyl” or “alkynyl” are each independently unsubstituted or substituted by at least one group of halogen, —OR₁, —SR₁, —(CO)OR₁, —(SO₂)R₁ or —N(R₁)₂; the “cycloalkyl”, “cycloalkylalkyl”, “cycloalkenyl”, “cycloalkenylalkyl”, “heterocyclyl”, “heterocyclylalkyl”, “aryl” or “arylalkyl” are each independently unsubstituted or substituted by at least one group selected from oxo, halogen, cyano, nitro, alkyl, alkenyl, alkynyl, cycloalkyl, haloalkyl, haloalkenyl, haloalkynyl, halocycloalkyl, cycloalkyl substituted by alkyl, —OR₁, —SR₁, —(CO)OR₁, —(SO₂)R₁, —N(R₁)₂ or —O-alkyl-(CO)OR₁, or two adjacent carbon atoms on the ring form a fused ring with —OCH₂CH₂— or —OCH₂O— that is unsubstituted or substituted by halogen;
X₁₃ and X₁₄ each independently represent hydrogen, halogen, cyano, alkoxy, alkoxyalkyl, alkylcarbonyl, alkoxycarbonyl, alkylsulfonyl, alkyl, alkenyl, alkynyl, cycloalkyl, cycloalkylalkyl, cycloalkenyl, cycloalkenylalkyl, aryl, arylalkyl, heterocyclyl or heterocyclylalkyl, or the CX₁₃X₁₄ group together forms an unsubstituted or substituted ring structure, or the NX₁₃X₁₄ group together forms an unsubstituted or substituted heterocyclyl with nitrogen atom at 1-position; wherein, the “alkyl”, “alkenyl” or “alkynyl” are each independently unsubstituted or substituted by at least one group of halogen, —OR₁, —SR₁, —(CO)OR₁, —(SO₂)R₁ or —N(R₁)₂; the “cycloalkyl”, “cycloalkylalkyl”, “cycloalkenyl”, “cycloalkenylalkyl”, “aryl”, “arylalkyl”, “heterocyclyl” or “heterocyclylalkyl” are each independently unsubstituted or substituted by at least one group selected from oxo, halogen, cyano, nitro, alkyl, alkenyl, alkynyl, cycloalkyl, haloalkyl, haloalkenyl, haloalkynyl, halocycloalkyl, cycloalkyl substituted by alkyl, —OR₁, —SR₁, —(CO)OR₁, —(SO₂)R₁, —N(R₁)₂ or —O-alkyl-(CO)OR₁, or two adjacent carbon atoms on the ring form a fused ring with —OCH₂CH₂— or —OCH₂O— that is unsubstituted or substituted by halogen;
R₁ each independently represents hydrogen, alkyl, haloalkyl, phenyl or phenyl substituted by at least one group selected from halogen, cyano, nitro, alkyl, haloalkyl, alkoxycarbonyl, alkylthio, alkylsulfonyl, alkoxy or haloalkoxy.

Preferably, X represents hydrogen, halogen, C1-C8 alkoxy or C1-C8 alkylthio;
Y represents halogen or halo C1-C8 alkyl;
Z and Z₁ each independently represent hydrogen, halogen, C1-C8 alkyl, C2-C8 alkenyl, C2-C8 alkynyl, C3-C8 cycloalkyl, halo C1-C8 alkyl or cyano;
Q and Q₁ each independently represent hydrogen, halogen, hydroxyl, cyano, amino, nitro, formyl, cyano C1-C8 alkyl, hydroxyl C1-C8 alkyl, C1-C8 alkyl, C2-C8 alkenyl, C2-C8 alkynyl, C3-C8 cycloalkyl, C3-C8 cycloalkyl C1-C8 alkyl, C1-C8 alkoxy, C1-C8 alkylthio, C1-C8 alkylamino, halo C1-C8 alkoxy, halo C1-C8 alkylthio, C1-C8 alkylcarbonyl, C1-C8 alkoxycarbonyl, C1-C8 alkoxy C1-C8 alkyl, halo C1-C8 alkoxy C1-C8 alkyl, C1-C8 alkylamino C1-C8 alkyl, aryl, heterocyclyl, aryl C1-C8 alkyl or heterocyclyl C1-C8 alkyl; wherein, the “C1-C8 alkyl”, “C2-C8 alkenyl” or “C2-C8 alkynyl” are each independently unsubstituted or substituted by at least one group of halogen, —OR₁, —SR₁, —(CO)OR₁, —(CO)N(R₁)₂, —(SO₂)R₁ or —N(R₁)₂; the “C3-C8 cycloalkyl”, “C3-C8 cycloalkyl C1-C8 alkyl”, “aryl”, “heterocyclyl”, “aryl C1-C8 alkyl” or “heterocyclyl C1-C8 alkyl” are each independently unsubstituted or substituted by at least one group selected from oxo, halogen, cyano, nitro, C1-C8 alkyl, C2-C8 alkenyl, C2-C8 alkynyl, C3-C8 cycloalkyl, halo C1-C8 alkyl, halo C2-C8 alkenyl, halo C2-C8 alkynyl, halo C3-C8 cycloalkyl, C3-C8 cycloalkyl substituted by C1-C8 alkyl, —OR₁, —SR₁, —(CO)OR₁, —(SO₂)R₁, —N(R₁)₂ or —O—(C1-C8 alkyl)-(CO)OR₁, or two adjacent carbon atoms on the ring form a fused ring with —OCH₂CH₂— or —OCH₂O— that is unsubstituted or substituted by halogen;
M represents hydrogen, C1-C8 alkyl, C2-C8 alkenyl, C2-C8 alkynyl, C3-C8 cycloalkyl, C3-C8 cycloalkenyl, heterocyclyl, aryl,

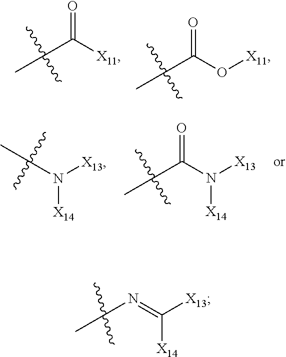

wherein, the “C1-C8 alkyl”, “C2-C8 alkenyl” or “C2-C8 alkynyl” are each independently unsubstituted or substituted by at least one group selected from halogen, cyano, nitro, C3-C8 cycloalkyl, tri(C1-C8)alkylsilyl, C3-C8 cycloalkenyl, heterocyclyl, aryl,

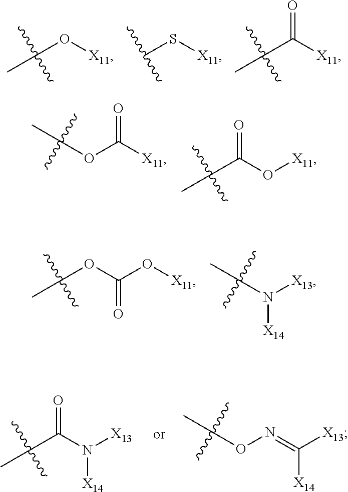

the “C3-C8 cycloalkyl”, “C3-C8 cycloalkenyl”, “heterocyclyl” or “aryl” are each independently unsubstituted or substituted by at least one group selected from oxo, halogen, cyano, nitro, C1-C8 alkyl, C2-C8 alkenyl, C2-C8 alkynyl, C3-C8 cycloalkyl, halo C1-C8 alkyl, halo C2-C8 alkenyl, halo C2-C8 alkynyl, halo C3-C8 cycloalkyl, C3-C8 cycloalkyl substituted by C1-C8 alkyl, —OR₁, —SR₁, —(CO)OR₁, —(SO₂)R₁, —N(R₁)₂ or —O—(C1-C8 alkyl)-(CO)OR₁, or two adjacent carbon atoms on the ring form a fused ring with —OCH₂CH₂— or —OCH₂O— that is unsubstituted or substituted by halogen;
R represents hydrogen, C1-C8 alkyl, C2-C8 alkenyl, C2-C8 alkynyl, C3-C8 cycloalkyl, C3-C8 cycloalkenyl, heterocyclyl, aryl,

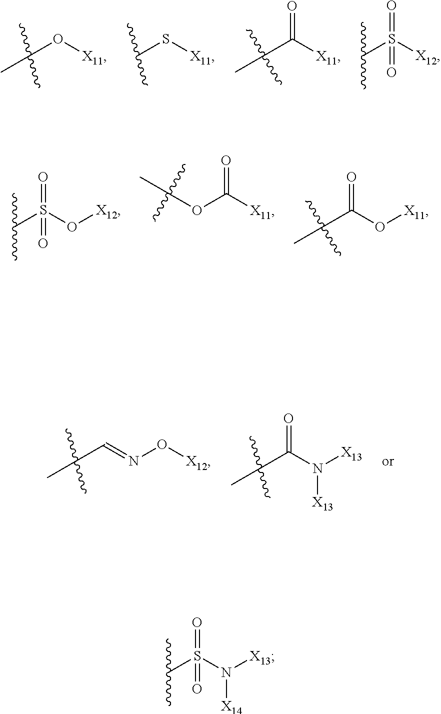

wherein, the “C1-C8 alkyl”, “C2-C8 alkenyl” or “C2-C8 alkynyl” are each independently unsubstituted or substituted by at least one group selected from halogen, cyano, nitro, C3-C8 cycloalkyl, tri(C1-C8)alkylsilyl, C3-C8 cycloalkenyl, heterocyclyl, aryl,

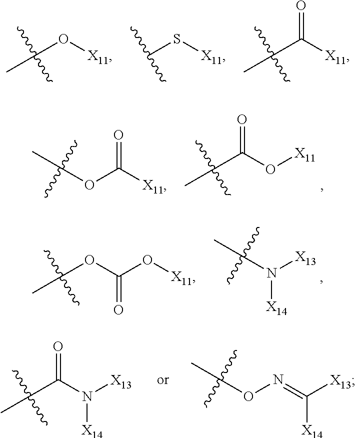

the “C3-C8 cycloalkyl”, “C3-C8 cycloalkenyl”, “heterocyclyl” or “aryl” are each independently unsubstituted or substituted by at least one group selected from oxo, halogen, cyano, nitro, C1-C8 alkyl, C2-C8 alkenyl, C2-C8 alkynyl, C3-C8 cycloalkyl, halo C1-C8 alkyl, halo C2-C8 alkenyl, halo C2-C8 alkynyl, halo C3-C8 cycloalkyl, C3-C8 cycloalkyl substituted by C1-C8 alkyl, —OR₁, —SR₁, —(CO)OR₁, —(SO₂)R₁, —N(R₁)₂ or —O—(C1-C8 alkyl)-(CO)OR₁, or two adjacent carbon atoms on the ring form a fused ring with —OCH₂CH₂— or —OCH₂O— that is unsubstituted or substituted by halogen;
X₁₁ each represents hydrogen, C1-C8 alkyl, C2-C8 alkenyl, C2-C8 alkynyl, C3-C8 cycloalkyl, C3-C8 cycloalkyl C1-C8 alkyl, C3-C8 cycloalkenyl, C3-C8 cycloalkenyl C1-C8 alkyl, heterocyclyl, heterocyclyl C1-C8 alkyl, aryl or aryl C1-C8 alkyl; wherein, the “C1-C8 alkyl”, “C2-C8 alkenyl” or “C2-C8 alkynyl” are each independently unsubstituted or substituted by at least one group of halogen, —OR₁, —SR₁, —(CO)OR₁, —(SO₂)R₁ or —N(R₁)₂; the “C3-C8 cycloalkyl”, “C3-C8 cycloalkyl C1-C8 alkyl”, “C3-C8 cycloalkenyl”, “C3-C8 cycloalkenyl C1-C8 alkyl”, “heterocyclyl”, “heterocyclyl C1-C8 alkyl”, “aryl” or “aryl C1-C8 alkyl” are each independently unsubstituted or substituted by at least one group selected from oxo, halogen, cyano, nitro, C1-C8 alkyl, C2-C8 alkenyl, C2-C8 alkynyl, C3-C8 cycloalkyl, halo C1-C8 alkyl, halo C2-C8 alkenyl, halo C2-C8 alkynyl, halo C3-C8 cycloalkyl, C3-C8 cycloalkyl substituted by C1-C8 alkyl, —OR₁, —SR₁, —(CO)OR₁, —(SO₂)R₁, —N(R₁)₂ or —O—(C1-C8 alkyl)-(CO)OR₁, or two adjacent carbon atoms on the ring form a fused ring with —OCH₂CH₂— or —OCH₂O— that is unsubstituted or substituted by halogen;
X₁₂ each independently represents C1-C8 alkyl, C2-C8 alkenyl, C2-C8 alkynyl, C3-C8 cycloalkyl, C3-C8 cycloalkyl C1-C8 alkyl, C3-C8 cycloalkenyl, C3-C8 cycloalkenyl C1-C8 alkyl, heterocyclyl, heterocyclyl C1-C8 alkyl, aryl or aryl C1-C8 alkyl; wherein, the “C1-C8 alkyl”, “C2-C8 alkenyl” or “C2-C8 alkynyl” are each independently unsubstituted or substituted by at least one group of halogen, —OR₁, —SR₁, —(CO)OR₁, —(SO₂)R₁ or —N(R₁)₂; the “C3-C8 cycloalkyl”, “C3-C8 cycloalkyl C1-C8 alkyl”, “C3-C8 cycloalkenyl”, “C3-C8 cycloalkenyl C1-C8 alkyl”, “heterocyclyl”, “heterocyclyl C1-C8 alkyl”, “aryl” or “aryl C1-C8 alkyl” are each independently unsubstituted or substituted by at least one group selected from oxo, halogen, cyano, nitro, C1-C8 alkyl, C2-C8 alkenyl, C2-C8 alkynyl, C3-C8 cycloalkyl, halo C1-C8 alkyl, halo C2-C8 alkenyl, halo C2-C8 alkynyl, halo C3-C8 cycloalkyl, C3-C8 cycloalkyl substituted by C1-C8 alkyl, —OR₁, —SR₁, —(CO)OR₁, —(SO₂)R₁, —N(R₁)₂ or —O—(C1-C8 alkyl)-(CO)OR₁, or two adjacent carbon atoms on the ring form a fused ring with —OCH₂CH₂— or —OCH₂O— that is unsubstituted or substituted by halogen;
X₁₃ and X₁₄ each independently represent hydrogen, halogen, cyano, C1-C8 alkoxy, C1-C8 alkoxy C1-C8 alkyl, C1-C8 alkylcarbonyl, C1-C8 alkoxycarbonyl, C1-C8 alkylsulfonyl, C1-C8 alkyl, C2-C8 alkenyl, C2-C8 alkynyl, C3-C8 cycloalkyl, C3-C8 cycloalkyl C1-C8 alkyl, C3-C8 cycloalkenyl, C3-C8 cycloalkenyl C1-C8 alkyl, aryl, aryl C1-C8 alkyl, heterocyclyl or heterocyclyl C1-C8 alkyl, or the CX₁₃X₁₄ group together forms a 5-8 membered carbocycle or oxygen-, sulfur- or nitrogen-containing heterocycle, or the NX₁₃X₁₄ group together forms

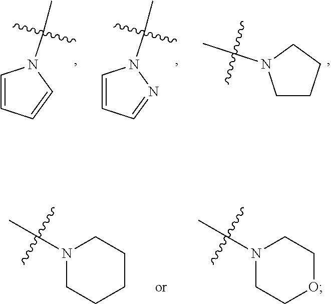

wherein, the “C1-C8 alkyl”, “C2-C8 alkenyl” or “C2-C8 alkynyl” are each independently unsubstituted or substituted by at least one group of halogen, —OR₁, —SR₁, —(CO)OR₁, —(SO₂)R₁ or —N(R₁)₂; the “C3-C8 cycloalkyl”, “C3-C8 cycloalkyl C1-C8 alkyl”, “C3-C8 cycloalkenyl”, “C3-C8 cycloalkenyl C1-C8 alkyl”, “aryl”, “aryl C1-C8 alkyl”, “heterocyclyl” or “heterocyclyl C1-C8 alkyl” are each independently unsubstituted or substituted by at least one group selected from oxo, halogen, cyano, nitro, C1-C8 alkyl, C2-C8 alkenyl, C2-C8 alkynyl, C3-C8 cycloalkyl, halo C1-C8 alkyl, halo C2-C8 alkenyl, halo C2-C8 alkynyl, halo C3-C8 cycloalkyl, C3-C8 cycloalkyl substituted by C1-C8 alkyl, —OR₁, —SR₁, —(CO)OR₁, —(SO₂)R₁, —N(R₁)₂ or —O—(C1-C8 alkyl)-(CO)OR₁, or two adjacent carbon atoms on the ring form a fused ring with —OCH₂CH₂— or —OCH₂O— that is unsubstituted or substituted by halogen; the “5-8 membered carbocycle or oxygen-, sulfur- or nitrogen-containing heterocycle” is unsubstituted or substituted by at least one group selected from C1-C8 alkyl, C1-C8 alkoxycarbonyl or benzyl, or forms a fused ring structure with aryl or heterocyclyl; the

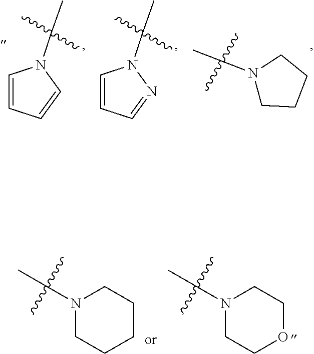

is unsubstituted or substituted by at least one group selected from oxo, C1-C8 alkyl or C1-C8 alkoxycarbonyl;
R₁ each independently represents hydrogen, C1-C8 alkyl, halo C1-C8 alkyl, phenyl or phenyl substituted by at least one group selected from halogen, cyano, nitro, C1-C8 alkyl, halo C1-C8 alkyl, C1-C8 alkoxycarbonyl, C1-C8 alkylthio, C1-C8 alkylsulfonyl, C1-C8 alkoxy or halo C1-C8 alkoxy.

More preferably, X represents hydrogen, halogen, C1-C6 alkoxy or C1-C6 alkylthio;
Y represents halogen or halo C1-C6 alkyl;
Z and Z₁ each independently represent hydrogen, halogen, C1-C6 alkyl, C2-C6 alkenyl, C2-C6 alkynyl, C3-C6 cycloalkyl, halo C1-C6 alkyl or cyano;
Q and Q₁ each independently represent hydrogen, halogen, hydroxyl, cyano, amino, nitro, formyl, cyano C1-C6 alkyl, hydroxyl C1-C6 alkyl, C1-C6 alkyl, C2-C6 alkenyl, C2-C6 alkynyl, C3-C6 cycloalkyl, C3-C6 cycloalkyl C1-C6 alkyl, C1-C6 alkoxy, C1-C6 alkylthio, C1-C6 alkylamino, halo C1-C6 alkoxy, halo C1-C6 alkylthio, C1-C6 alkylcarbonyl, C1-C6 alkoxycarbonyl, C1-C6 alkoxy C1-C6 alkyl, halo C1-C6 alkoxy C1-C6 alkyl, C1-C6 alkylamino C1-C6 alkyl, aryl, heterocyclyl, aryl C1-C6 alkyl or heterocyclyl C1-C6 alkyl; wherein, the “C1-C6 alkyl”, “C2-C6 alkenyl” or “C2-C6 alkynyl” are each independently unsubstituted or substituted by at least one group of halogen, —OR₁, —SR₁, —(CO)OR₁, —(CO)N(R₁)₂, —(SO₂)R₁ or —N(R₁)₂; the “C3-C6 cycloalkyl”, “C3-C6 cycloalkyl C1-C6 alkyl”, “aryl”, “heterocyclyl”, “aryl C1-C6 alkyl” or “heterocyclyl C1-C6 alkyl” are each independently unsubstituted or substituted by at least one group selected from oxo, halogen, cyano, nitro, C1-C6 alkyl, C2-C6 alkenyl, C2-C6 alkynyl, C3-C6 cycloalkyl, halo C1-C6 alkyl, halo C2-C6 alkenyl, halo C2-C6 alkynyl, halo C3-C6 cycloalkyl, C3-C6 cycloalkyl substituted by C1-C6 alkyl, —OR₁, —SR₁, —(CO)OR₁, —(SO₂)R₁, —N(R₁)₂ or —O—(C1-C6 alkyl)-(CO)OR₁, or two adjacent carbon atoms on the ring form a fused ring with —OCH₂CH₂— or —OCH₂O— that is unsubstituted or substituted by halogen;
M represents hydrogen, C1-C6 alkyl, C2-C6 alkenyl, C2-C6 alkynyl, C3-C6 cycloalkyl, C3-C6 cycloalkenyl, heterocyclyl, aryl,

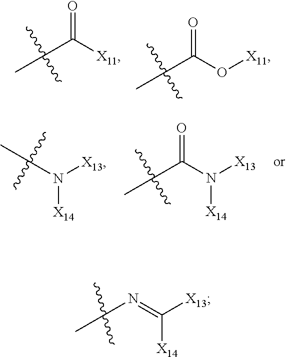

wherein, the “C1-C6 alkyl”, “C2-C6 alkenyl” or “C2-C6 alkynyl” are each independently unsubstituted or substituted by at least one group selected from halogen, cyano, nitro, C3-C6 cycloalkyl, tri(C1-C6)alkylsilyl, C3-C6 cycloalkenyl, heterocyclyl, aryl,

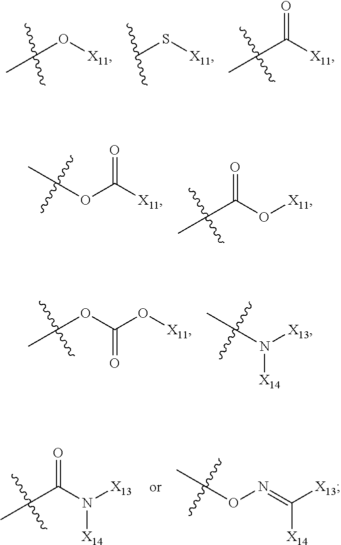

the “C3-C6 cycloalkyl”, “C3-C6 cycloalkenyl”, “heterocyclyl” or “aryl” are each independently unsubstituted or substituted by at least one group selected from oxo, halogen, cyano, nitro, C1-C6 alkyl, C2-C6 alkenyl, C2-C6 alkynyl, C3-C6 cycloalkyl, halo C1-C6 alkyl, halo C2-C6 alkenyl, halo C2-C6 alkynyl, halo C3-C6 cycloalkyl, C3-C6 cycloalkyl substituted by C1-C6 alkyl, —OR₁, —SR₁, —(CO)OR₁, —(SO₂)R₁, —N(R₁)₂ or —O—(C1-C6 alkyl)-(CO)OR₁, or two adjacent carbon atoms on the ring form a fused ring with —OCH₂CH₂— or —OCH₂O— that is unsubstituted or substituted by halogen;
R represents hydrogen, C1-C6 alkyl, C2-C6 alkenyl, C2-C6 alkynyl, C3-C6 cycloalkyl, C3-C6 cycloalkenyl, heterocyclyl, aryl,

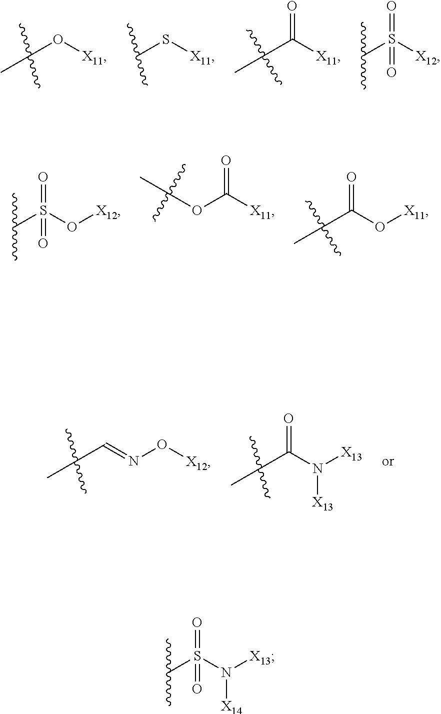

wherein, the “C1-C6 alkyl”, “C2-C6 alkenyl” or “C2-C6 alkynyl” are each independently unsubstituted or substituted by at least one group selected from halogen, cyano, nitro, C3-C6 cycloalkyl, tri(C1-C6)alkylsilyl, C3-C6 cycloalkenyl, heterocyclyl, aryl,

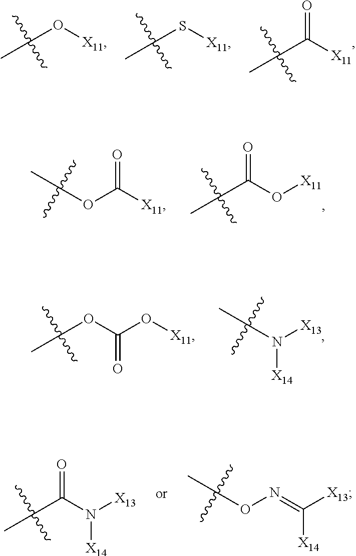

the “C3-C6 cycloalkyl”, “C3-C6 cycloalkenyl”, “heterocyclyl” or “aryl” are each independently unsubstituted or substituted by at least one group selected from oxo, halogen, cyano, nitro, C1-C6 alkyl, C2-C6 alkenyl, C2-C6 alkynyl, C3-C6 cycloalkyl, halo C1-C6 alkyl, halo C2-C6 alkenyl, halo C2-C6 alkynyl, halo C3-C6 cycloalkyl, C3-C6 cycloalkyl substituted by C1-C6 alkyl, —OR₁, —SR₁, —(CO)OR₁, —(SO₂)R₁, —N(R₁)₂ or —O—(C1-C6 alkyl)-(CO)OR₁, or two adjacent carbon atoms on the ring form a fused ring with —OCH₂CH₂— or —OCH₂O— that is unsubstituted or substituted by halogen;
X₁₁ each independently represents hydrogen, C1-C6 alkyl, C2-C6 alkenyl, C2-C6 alkynyl, C3-C6 cycloalkyl, C3-C6 cycloalkyl C1-C6 alkyl, C3-C6 cycloalkenyl, C3-C6 cycloalkenyl C1-C6 alkyl, heterocyclyl, heterocyclyl C1-C6 alkyl, aryl or aryl C1-C6 alkyl; wherein, the “C1-C6 alkyl”, “C2-C6 alkenyl” or “C2-C6 alkynyl” are each independently unsubstituted or substituted by at least one group of halogen, —OR₁, —SR₁, —(CO)OR₁, —(SO₂)R₁ or —N(R₁)₂; the “C3-C6 cycloalkyl”, “C3-C6 cycloalkyl C1-C6 alkyl”, “C3-C6 cycloalkenyl”, “C3-C6 cycloalkenyl C1-C6 alkyl”, “heterocyclyl”, “heterocyclyl C1-C6 alkyl”, “aryl” or “aryl C1-C6 alkyl” are each independently unsubstituted or substituted by at least one group selected from oxo, halogen, cyano, nitro, C1-C6 alkyl, C2-C6 alkenyl, C2-C6 alkynyl, C3-C6 cycloalkyl, halo C1-C6 alkyl, halo C2-C6 alkenyl, halo C2-C6 alkynyl, halo C3-C6 cycloalkyl, C3-C6 cycloalkyl substituted by C1-C6 alkyl, —OR₁, —SR₁, —(CO)OR₁, —(SO₂)R₁, —N(R₁)₂ or —O—(C1-C6 alkyl)-(CO)OR₁, or two adjacent carbon atoms on the ring form a fused ring with —OCH₂CH₂ or —OCH₂O— that is unsubstituted or substituted by halogen;
X₁₂ each independently represents C1-C6 alkyl, C2-C6 alkenyl, C2-C6 alkynyl, C3-C6 cycloalkyl, C3-C6 cycloalkyl C1-C6 alkyl, C3-C6 cycloalkenyl, C3-C6 cycloalkenyl C1-C6 alkyl, heterocyclyl, heterocyclyl C1-C6 alkyl, aryl or aryl C1-C6 alkyl; wherein, the “C1-C6 alkyl”, “C2-C6 alkenyl” or “C2-C6 alkynyl” are each independently unsubstituted or substituted by at least one group of halogen, —OR₁, —SR₁, —(CO)OR₁, —(SO₂)R₁ or —N(R₁)₂; the “C3-C6 cycloalkyl”, “C3-C6 cycloalkyl C1-C6 alkyl”, “C3-C6 cycloalkenyl”, “C3-C6 cycloalkenyl C1-C6 alkyl”, “heterocyclyl”, “heterocyclyl C1-C6 alkyl”, “aryl” or “aryl C1-C6 alkyl” are each independently unsubstituted or substituted by at least one group selected from oxo, halogen, cyano, nitro, C1-C6 alkyl, C2-C6 alkenyl, C2-C6 alkynyl, C3-C6 cycloalkyl, halo C1-C6 alkyl, halo C2-C6 alkenyl, halo C2-C6 alkynyl, halo C3-C6 cycloalkyl, C3-C6 cycloalkyl substituted by C1-C6 alkyl, —OR₁, —SR₁, —(CO)OR₁, —(SO₂)R₁, —N(R₁)₂ or —O—(C1-C6 alkyl)-(CO)OR₁, or two adjacent carbon atoms on the ring form a fused ring with —OCH₂CH₂— or —OCH₂O— that is unsubstituted or substituted by halogen;
X₁₃ and X₁₄ each independently represent hydrogen, halogen, cyano, C1-C6 alkoxy, C1-C6 alkoxy C1-C6 alkyl, C1-C6 alkylcarbonyl, C1-C6 alkoxycarbonyl, C1-C6 alkylsulfonyl, C1-C6 alkyl, C2-C6 alkenyl, C2-C6 alkynyl, C3-C6 cycloalkyl, C3-C6 cycloalkyl C1-C6 alkyl, C3-C6 cycloalkenyl, C3-C6 cycloalkenyl C1-C6 alkyl, aryl, aryl C1-C6 alkyl, heterocyclyl or heterocyclyl C1-C6 alkyl, or the group CX₁₃X₁₄ together forms a 5-8 membered carbocycle or oxygen-, sulfur- or nitrogen-containing heterocycle, or the group NX₁₃X₁₄ together forms

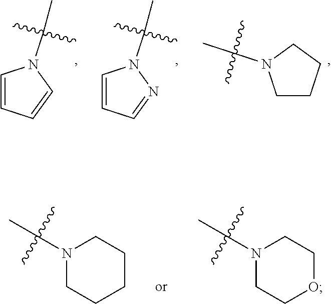

wherein, the “C1-C6 alkyl”, “C2-C6 alkenyl” or “C2-C6 alkynyl” are each independently unsubstituted or substituted by at least one group of halogen, —OR₁, —SR₁, —(CO)OR₁, —(SO₂)R₁ or —N(R₁)₂; the “C3-C6 cycloalkyl”, “C3-C6 cycloalkyl C1-C6 alkyl”, “C3-C6 cycloalkenyl”, “C3-C6 cycloalkenyl C1-C6 alkyl”, “aryl”, “aryl C1-C6 alkyl”, “heterocyclyl” or “heterocyclyl C1-C6 alkyl” are each independently unsubstituted or substituted by at least one group selected from oxo, halogen, cyano, nitro, C1-C6 alkyl, C2-C6 alkenyl, C2-C6 alkynyl, C3-C6 cycloalkyl, halo C1-C6 alkyl, halo C2-C6 alkenyl, halo C2-C6 alkynyl, halo C3-C6 cycloalkyl, C3-C6 cycloalkyl substituted by C1-C6 alkyl, —OR₁, —SR₁, —(CO)OR₁, —(SO₂)R₁, —N(R₁)₂ or —O—(C1-C6 alkyl)-(CO)OR₁, or two adjacent carbon atoms on the ring form a fused ring with —OCH₂CH₂— or —OCH₂O— that is unsubstituted or substituted by halogen; the “5-8 membered carbocycle or oxygen-, sulfur- or nitrogen-containing heterocycle” is unsubstituted or substituted by at least one group selected from C1-C6 alkyl, C1-C6 alkoxycarbonyl or benzyl, or forms a fused ring structure with aryl or heterocyclyl; the

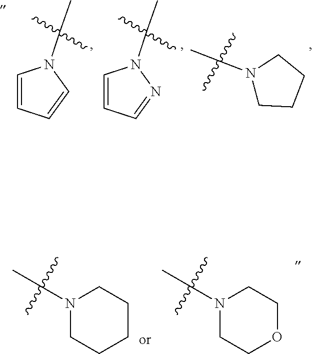

is unsubstituted or substituted by at least one group selected from oxo, C1-C6 alkyl or C1-C6 alkoxycarbonyl;
R₁ each independently represents hydrogen, C1-C6 alkyl, halo C1-C6 alkyl, phenyl or phenyl substituted by at least one group selected from halogen, cyano, nitro, C1-C6 alkyl, halo C1-C6 alkyl, C1-C6 alkoxycarbonyl, C1-C6 alkylthio, C1-C6 alkylsulfonyl, C1-C6 alkoxy or halo C1-C6 alkoxy.

In one specific embodiment, X represents hydrogen, fluorine, chlorine, methoxy or methylthio;
Y represents chlorine or fluorine;
Z represents hydrogen, methyl, ethyl, isopropyl, chlorine, trifluoromethyl or vinyl;
Z₁ represents hydrogen or chlorine;
Q represents hydrogen, methyl, isopropyl, cyclopropyl, methoxy, trifluoromethyl, benzyl,

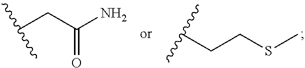

Q₁ represents hydrogen;
P represents CH or N;
P₁ represents CH or N;
M represents hydrogen, methyl, ethyl, allyl, propargyl,

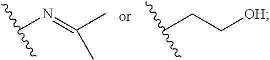

R represents hydrogen or methyl.

In another specific embodiment, P and P₁ are not simultaneously N.

In another specific embodiment, P represents CH; P₁ represents CH; Q₁ represents hydrogen; Z₁ represents hydrogen.

In another specific embodiment, P₁ represents CH; Q₁ represents hydrogen; Z₁ represents hydrogen.

In the definitions of the compounds represented by the above-mentioned general formulas and all of the following structural formulas, the technical terms used, whether used alone or used in compound words, represent the following substituent groups: an alkyl group which has more than two carbon atoms and may be linear or branched. For example, the alkyl in the compound word “—O-(alkyl)-COOR₁” may be —CH₂—, —CH₂CH₂—, —CH(CH₃)—, —C(CH₃)₂—, etc. An alkyl group is, for example, C1 alkyl: methyl; C2 alkyl: ethyl; C3 alkyl: propyl such as n-propyl or isopropyl; C4 alkyl: butyl such as n-butyl, isobutyl, tert-butyl or 2-butyl; C5 alkyl: pentyl such as n-pentyl; C6 alkyl: hexyl such as n-hexyl, isohexyl and 1,3-dimethylbutyl. Similarly, an alkenyl is, for example, vinyl, allyl, 1-methylprop-2-en-1-yl, 2-methylprop-2-en-1-yl, but-2-en-1-yl, but-3-en-1-yl, 1-methylbut-3-en-1-yl and 1-methylbut-2-en-1-yl. An alkynyl is, for example, ethynyl, propargyl, but-2-yn-1-yl, but-3-yn-1-yl, 1-methylbut-3-yn-1-yl. A multiple bond may be placed at any position of each unsaturated group. A cycloalkyl is a saturated carbocyclic ring system having, for example, three to six carbon atoms, such as cyclopropyl, cyclobutyl, cyclopentyl or cyclohexyl. Similarly, a cycloalkenyl is a monocyclic alkenyl having, for example, three to six carbon ring members, such as cyclopropenyl, cyclobutenyl, cyclopentenyl, and cyclohexenyl, wherein a double bond can be at any position. A halogen is fluorine, chlorine, bromine or iodine.

Unless otherwise specified, the “aryl” of the present invention includes, but is not limited to, phenyl, naphthyl,

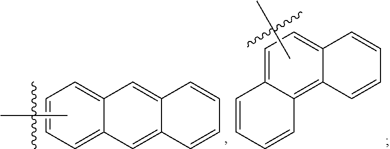

the “heterocyclyl” not only includes, but is not limited to, saturated or unsaturated non-aromatic cyclic groups

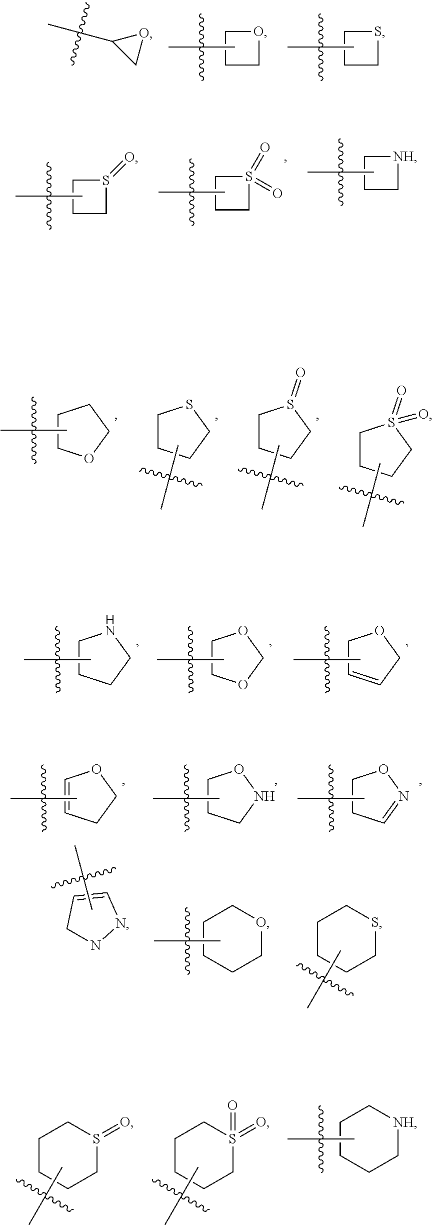

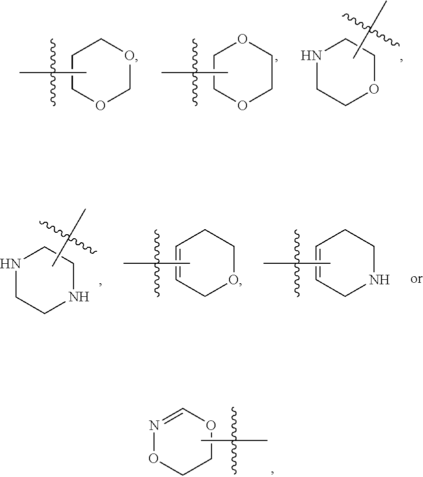

etc., but also includes, but is not limited to, “heteroaryl”, that is an aromatic cyclic group which has, for example, 3 to 6 ring atoms and a benzo ring fused with optionally. And 1 to 4 (for example, 1, 2, 3 or 4 heteroatoms of the ring atoms are selected from oxygen, nitrogen and sulfur, for example,

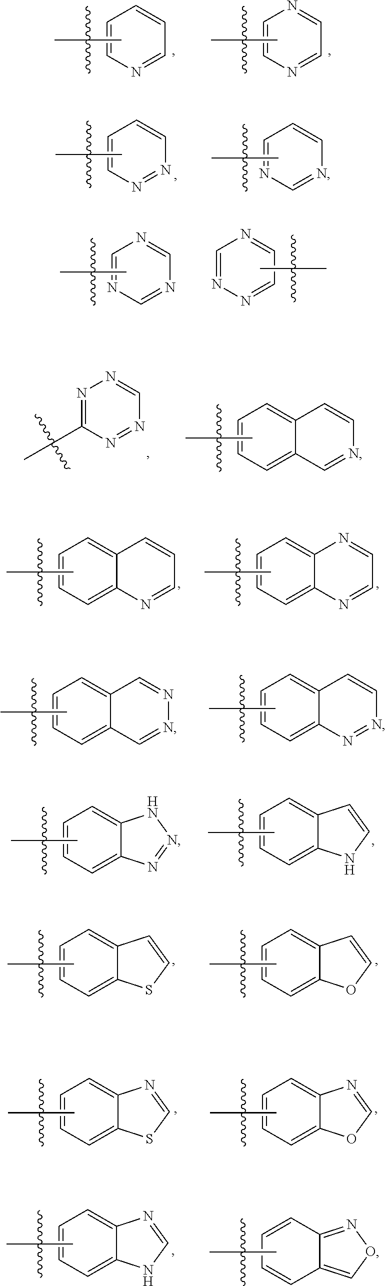

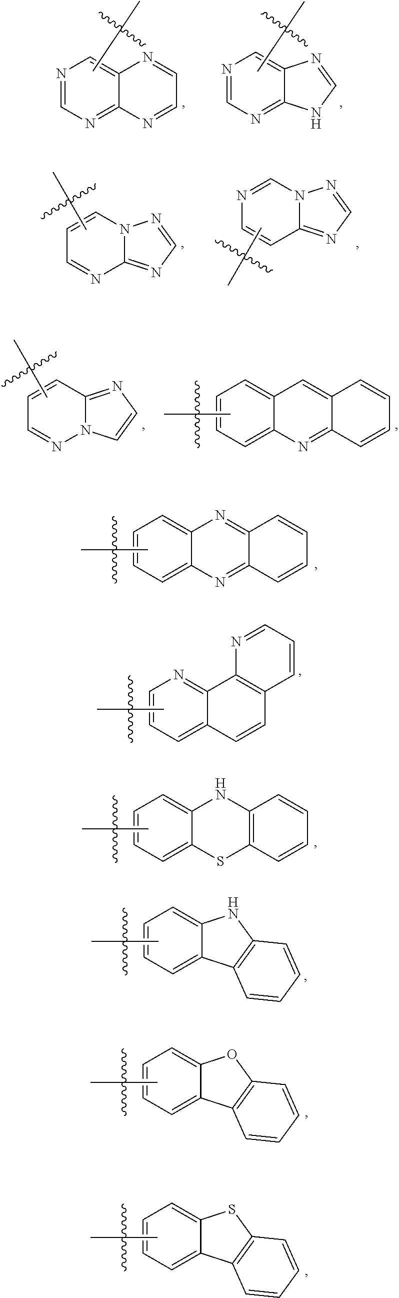

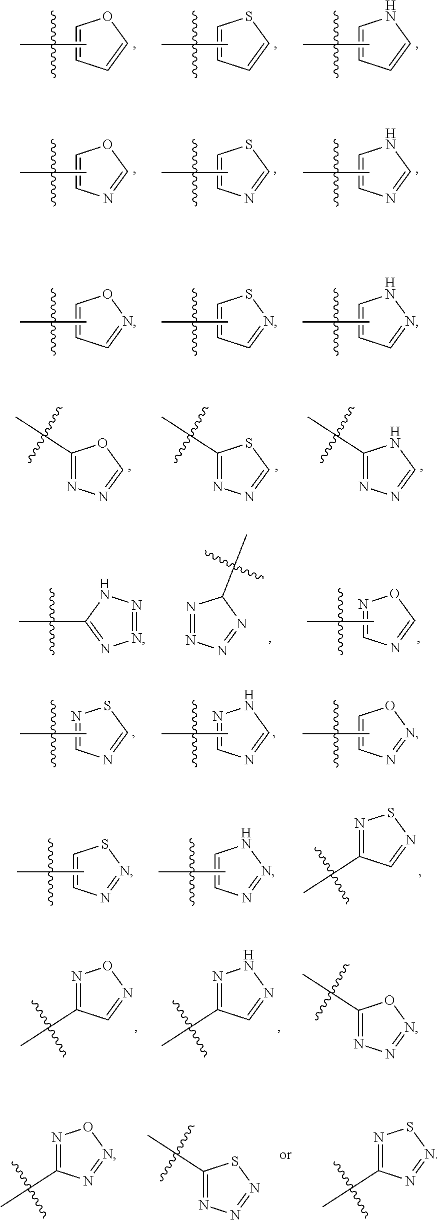

If one group is substituted by a group, which should be understood to mean that the group is substituted by one or more same or different groups selected from the mentioned groups. In addition, the same or different substitution characters contained in the same or different substituent groups are independently selected, which may be the same or different. This is also applicable to ring systems formed by different atoms and units. Meanwhile, the scope of the claims will exclude those compounds chemically unstable under standard conditions which are known to those skilled in the art.

In addition, unless specifically defined, the term “substituted by at least one group” herein refers to being substituted by, for example, 1, 2, 3, 4 or 5 groups; a group (including heterocyclyl, aryl, etc.) without being specified a linking site may be connected at any site, including a site connected to C or N; if it is substituted, the substituent group may also be substituted at any site as long as the valence bond theory is complied with. For example, a heteroaryl substituted by 1 methyl

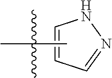

may represent

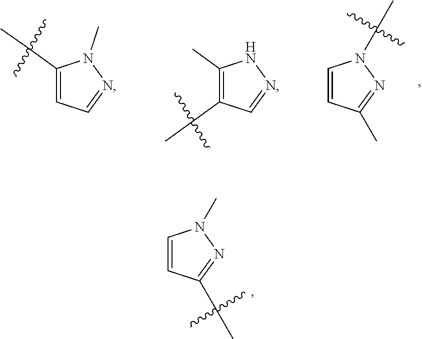

etc.

The present invention provides an isoxazoline-substituted benzamide compound with a chiral center as shown in Formula I′, a stereoisomer and an agriculturally or veterinarily acceptable salt thereof:

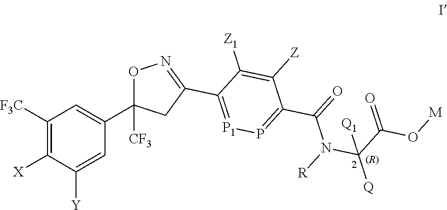

wherein, the definitions of substituent groups X, Y, Z, Z₁, P, P₁, R, Q, Q₁ and M are as described above, and Q and Q₁ are different; the carbon atom at position 2 is a chiral center; based on the contents of stereoisomers that have R- and S-configurations at this position, it has a stereochemical purity of 60-100% (R), preferably 70-100% (R), more preferably 80-100% (R), further preferably 90-100% (R), even further preferably 95-100% (R).

The present invention also provides an isoxazoline-substituted benzamide with a chiral center as shown in Formula I″, a stereoisomer and an agriculturally or veterinarily acceptable salt thereof:

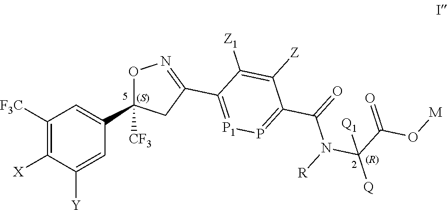

wherein, the definitions of substituent groups X, Y, Z, Z₁, P, P₁, R, Q, Q₁ and M are as described above, and Q and Q₁ are different;
based on the contents of stereoisomers that have R- and S-configurations at position 5, it has a stereochemical purity of 60-100% (S), preferably 70-100% (S), more preferably 80-100% (S), further preferably 90-100% (S), even further preferably 95-100% (S);
and the carbon atom at position 2 is a chiral center; based on the contents of stereoisomers that have R- and S-configurations at this position, it has a stereochemical purity of 60-100% (R), preferably 70-100% (R), more preferably 80-100% (R), further preferably 90-100% (R), even further preferably 95-100% (R).

Among them, “stereochemical purity” refers to the percentage of the amount of the stereoisomers to the total amount of stereoisomers with chiral centers.

In the present invention, the stereochemical configurations at position 5 and 2 in Formula I′ and Formula I″ are respectively determined as the main (5S) and (2R) according to Cahn-Ingold-Prelog system. However, the subject matter of the present invention also relates to all the stereoisomers at other positions encompassed by Formula I, I′ and I″, and mixtures thereof. Such compounds of Formula I, I′ and I″ contain, for example, one or more additional asymmetric carbon atoms or other double bonds that are not specified in Formula I, I′ and I″. It is to be understood that the present invention comprises pure isomers and mixtures thereof of pure isomers enriched to varying degrees, wherein, the asymmetric carbon atom at the labeled position 5 is in S-configuration and/or the asymmetric carbon atom at the labeled position 2 is in R-configuration; or in the mixture, a compound or a compound of the same chemical structure has the configuration at the labeled position, or is present in such a proportion where the compound with the configuration is predominantly present (at least 60% with the configuration); meanwhile, other asymmetric carbon atoms may exist in racemic forms, or may be resolved to varying degrees. As long as the stereochemical configuration conditions at the labeled position are met, possible stereoisomers defined by particular spatial forms, such as enantiomers, diastereoisomers, Z- and E-isomers, are all included in Formula I, I′ and I″, and may be obtained from mixtures of stereoisomers using conventional methods, or may be prepared by stereoselective reactions in combination with the use of stereochemically pure starting materials.

The present invention also includes any keto-enol tautomeric form, a mixture and salt thereof, if various functional groups are present.

Stereoisomers may be obtained from the mixtures prepared by optical resolution. Stereoisomers can also be selectively prepared by employing stereoselective reactions and using optically active starting materials and/or auxiliaries. For optical resolution, conventional methods can often be employed (see Textbooks of Stereochemistry), such as those described below for resolving the mixtures into diastereoisomers, for example, physical methods, e.g., crystallization, chromatography, particularly column chromatography and high pressure liquid chromatography, distillation methods carried out under reduced pressure as needed, extraction and other methods. Chromatographic separation on a chiral solid phase is usually adopted to separate residual mixtures of enantiomers. Those suitable for preparation amount or for use on an industrial scale are methods such as crystallization of diastereomeric salts, which can be obtained from compounds using optically active acids, and if acidic groups are present, optically active bases can be utilized as needed.

Salts of compounds of Formula I, I′ and I″ are preferably agriculturally or veterinary acceptable salts. They may be formed by conventional manners, for example, by reacting the compounds with the acids of the anion if the compounds of Formula I, I′ and I″ have basic functional groups, or by reacting the acidic compounds of Formula I, I′ and I″ with suitable bases.

Suitable agriculturally acceptable salts are especially the salts of those cations or the acid addition salts of those acids whose cations and anions, respectively, do not have any adverse effect on the action on the compounds according to the present invention. Suitable cations are in particular ions of alkali metals, preferably lithium, sodium and potassium; ions of alkaline earth metals, preferably calcium, magnesium and barium; ions of transition metals, preferably manganese, copper, zinc and iron; and also ammonium (NH₄⁺) and substituted ammonium in which 1 to 4 of the hydrogen atoms are replaced with C₁-C₄ alkyl, C₁-C₄ hydroxyalkyl, C₁-C₄ alkoxy, C₁-C₄ alkoxy-C₁-C₄ alkyl, hydroxy-C₁-C₄ alkoxy-C₁-C₄ alkyl, phenyl or benzyl. Examples of substituted ammonium include methylammonium, isopropylammonium, dimethylammonium, diisopropylammonium, trimethylammonium, tetramethylammonium, tetraethylammonium, tetrabutylammonium, 2-hydroxyethylammonium, 2-(2-hydroxyethoxy)ethylammonium, bis(2-hydroxyethyl)ammonium, benzyltrimethylammonium and benzyltriethylammonium; in addition, phosphonium ions, sulfonium ions, preferably tris(C₁-C₄ alkyl)sulfonium, and sulfoxonium ions, preferably tris(C₁-C₄ alkyl)sulfoxonium.

Anions of useful acid addition salts are mainly chloride, bromide, fluoride, hydrogen sulfate, sulfate, dihydrogen phosphate, hydrogen phosphate, phosphate, nitrate, hydrogen carbonate, carbonate, hexafluorosilicate, hexafluorophosphate, benzoate and anions of C₁-C₄ alkanoic acids, preferably formate, acetate, propionate and butyrate. They can be formed by reacting the compounds of Formula I, I′ and I″ with an acid of the corresponding anion, preferably hydrochloric acid, hydrobromic acid, sulfuric acid, phosphoric acid or nitric acid.

The term “veterinarily acceptable salts” refers to those salts of cations or anions known and accepted in the established field of veterinary salts. For example, suitable acid addition salts formed by the compounds of Formula I, I′ and I″ containing a basic nitrogen atom, such as amino, include salts of inorganic acids such as hydrochloride, sulfate, phosphate and nitrate, as well as salts of organic acids such as salts of acetic acid, maleic acid, dimaleic acid, fumaric acid, difumaric acid, methanesulfenic acid, methanesulfonic acid and succinic acid.

The compounds of the present invention, including its salts, stereoisomers and tautomers, are especially suitable for effectively controlling invertebrate pests. The “invertebrate pest” includes arthropods, gastropods, nematodes and helminths of economic importance as pests. The term “arthropod” includes insects, mites, spiders, scorpions, centipedes, millipedes, pill bugs and animals in the Class Symphyla. The term “gastropod” includes snails, slugs and other animals in the Order Stylommatophora. The term “nematode” includes all members in the Class Nematoda. It is particularly suitable for effectively controlling or killing the following pests: lepidopterans (Lepidoptera), beetles (Coleoptera), flies, mosquitoes (Diptera), thrips (Thysanoptera), termites (Isoptera), cockroaches (Blattaria-Blattodea), bedbugs, aphids, leafhoppers, whiteflies, scale insects, cicadae (Hemiptera), ants, honeybees, wasps, sawflies (Hymenoptera), crickets, grasshoppers, locusts (Orthoptera), Arachnida, fleas (Siphonaptera), silverfish, firebrat (Thysanura), centipedes (Chilopoda), millipedes (Diplopoda), earwigs (Dermaptera), louse (Phthiraptera), springtails (Collembola), etc. They may attack plants, thereby causing significant damage to the attacked plants; and may infest animals, in particular warm-blooded animals such as mammals or birds or other higher animals such as reptiles, amphibians or fish, thereby resulting in ectoparasites that cause significant damage to the infested animals.

The present invention also relates a preparation method of the isoxazoline-substituted benzamide compound, its stereoisomer and agriculturally or veterinary acceptable salt, comprising the following steps:

In the presence of a condensing agent, the compound as shown in Formula I is prepared by reacting the compound shown in Formula II with the compound shown in Formula III, and the chemical reaction equation is as follows:

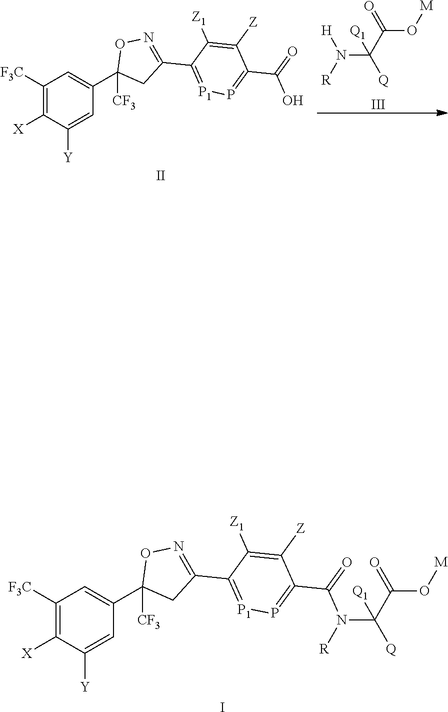

wherein, the substituent groups X, Y, Z, Z₁, P, P₁, R, Q, Q₁ and M are as defined before.

Preferably, the condensing agent is PyBOP, HATU, HOBt-EDCI, CDI, DCC or DBU, etc.; the reaction is carried out in the presence of a base and a solvent.

The solvent is selected from one or more mixed solvents of dichloromethane, dichloroethane, acetonitrile, N,N-dimethylformamide, N,N-dimethylacetamide, dimethylsulfoxide, tetrahydrofuran, toluene and xylene; the base is selected from one or more of triethylamine, trimethylamine, DIPEA and NMM.

In one specific embodiment, the compound shown in Formula II is prepared by hydrolysis of the compound shown in Formula IV, and the chemical reaction equation is as follows:

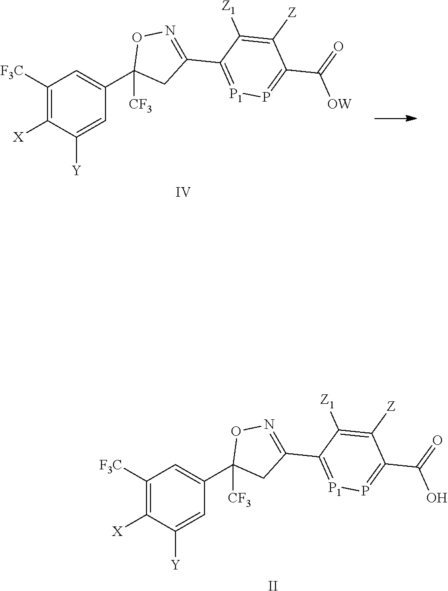

The compound shown in Formula IV is prepared by cycloaddition reaction between the compound shown in Formula V and the compound shown in Formula VI, and the chemical reaction equation is as follows:

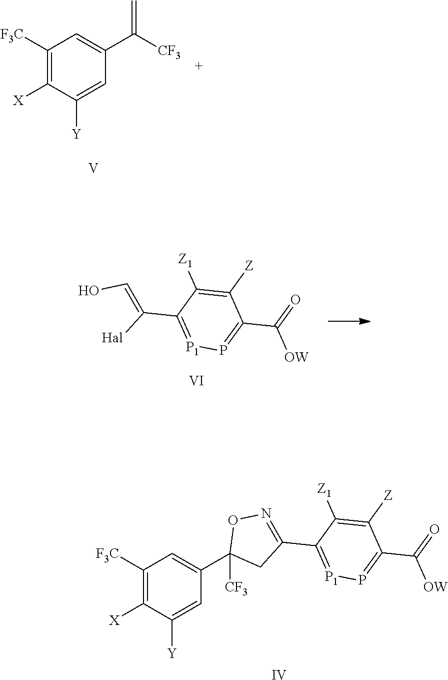

wherein, Hal represents halogen, and W represents C1-C6 alkyl or phenyl.

In addition, the compounds of the above-mentioned general formulas can be prepared with reference to the methods set forth in CN111936492A, WO2005/085216, etc.

The present invention also relates to an insecticidal composition, comprising biologically effective doses of at least one of the isoxazoline-substituted benzamide compound, its stereoisomer, and agriculturally or veterinary acceptable salt. Preferably, a formulation auxiliary is included as well.

Such compositions may comprise an individual active compound of the present invention or a mixture of several active compounds of the present invention. The composition of the present invention may comprise an individual isomer, or a mixture of isomers, or salts, as well as an individual tautomer or a mixture of tautomers.

Furthermore, the composition of the present invention may also comprise an insecticide or acaricide having a mode of action classified according to the Insecticide Resistance Action Committee (IRAC) regulations as an active ingredient, and apparent synergistic and enhanced effects will be produced by applying in combination.

Examples of insecticides or acaricides having modes of action classified according to the IRAC regulations include (1A) acetylcholinesterase (AChE) inhibitors (carbamates), (1B) acetylcholinesterase (AChE) inhibitors (organophosphates), (2) GABA-gated chloride channel blockers, (3A) sodium channel modulators (pyrethroids), (3B) sodium channel modulators (DDT), (4) nicotinic acetylcholine receptor (nAChR) competitive modulators, (5) nicotinic acetylcholine receptor (nAChR) allosteric modulators, (6) glutamate-gated chloride channel (GluCl) allosteric modulators, (7) juvenile hormone mimics, (8) miscellaneous non-specific (multi-site) inhibitors, (9) chordotonal organ TRPV channel modulators, (10) mite growth inhibitors, (11) microbial disruptors of insect midgut membranes, (12) inhibitors of mitochondrial ATP synthase, (13) uncouplers of oxidative phosphorylation via disruption of the proton gradient, (14) nicotinic acetylcholine receptor (nAChR) channel blockers, (15) inhibitors of chitin biosynthesis, type 0, (16) inhibitors of chitin biosynthesis, type 1, (17) moulting disruptors, (18) ecdysone receptor agonists, (19) octopamine receptor agonists, (20) mitochondrial complex III electron transport inhibitors, (21) mitochondrial complex I electron transport inhibitors (METI), (22) voltage-dependent sodium channel blockers, (23) inhibitors of acetyl CoA carboxylase, (24) mitochondrial complex IV electron transport inhibitors, (25) mitochondrial complex II electron transport inhibitors, (28) ryanodine receptor modulators, (29) chordotonal organ modulators-undefined target site, (30) GABA-gated chloride channel allosteric modulators, and compounds of unknown or uncertain modes of action (UN).

Specific examples of ingredient B are illustrated below.

### (1A) Acetylcholinesterase (AChE) Inhibitors (Carbamates)

Alanycarb, Aldicarb, Bendiocarb, Benfuracarb, Butocarboxim, Butoxycarboxim, Carbaryl, Carbofuran, Carbosulfan, Ethiofencarb, Fenobucarb, Formetanate, Furathiocarb, Isoprocarb, Methiocarb, Methomyl, Metolcarb, Oxamyl, Pirimicarb, Propoxur, Thiodicarb, Thiofanox, Triazamate, Trimethacarb, XMC and Xylylcarb.

Aldoxycarb, Allyxycarb, Aminocarb, Bufencarb, Cloethocarb, Fenothiocarb and Promecarb.

### (1B) Acetylcholinesterase (AChE) Inhibitors (Organophosphates)

Acephate, Azamethiphos, Azinphos-ethyl, Azinphos-methyl, Cadusafos, Chlorethoxyfos, Chlorfenvinphos, Chlormephos, Chlorpyrifos, Chlorpyrifos-methyl, Coumaphos, Cyanophos, Demeton-S-methyl, Diazinon, Dichlorvos/DDVP, Dicrotophos, Dimethoate, Dimethylvinphos, Disulfoton, EPN, Ethion, Ethoprophos, Famphur, Fenamiphos, Fenitrothion, Fenthion, Fosthiazate, Heptenophos, Imicyafos, Isofenphos, Isopropyl O-(methoxyaminothio-phosphoryl)salicylate, Isoxathion, Malathion, Mecarbam, Methamidophos, Methidathion, Mevinphos, Monocrotophos, Naled, Omethoate, Oxydemeton-methyl, Parathion, Parathion-methyl, Phenthoate, Phorate, Phosalone, Phosmet, Phosphamidon, Phoxim, Pirimiphos-methyl, Profenofos, Propetamphos, Prothiofos, Pyraclofos, Pyridaphenthion, Quinalphos, Sulfotep, Tebupirimfos, Temephos, Terbufos, Tetrachlorvinphos, Thiometon, Triazophos, Trichlorfon, Vamidothion, Bromophos-ethyl, Cyanofenphos, Demeton-S-methylsulfone, Dialifos, Dichlofenthion, Dioxabenzofos, Etrimfos, Fensulfothion, Fonofos, Formothion, Iodofenphos, Isazofos, Isocarbofos, Methacrifos, Phosphocarb, Pirimiphos-ethyl, Propaphos, Prothoate and Sulprofos.

### (2) GABA-Gated Chloride Channel Blockers

Chlordane, Endosulfan, Ethiprole, Fipronil, Acetoprole, Camphechlor, Dienochlor, Heptachlor, Pyrafluprole, Pyriprole and Flufiprole.

### (3A) Sodium Channel Modulators (Pyrethroids)

Acrinathrin, Allethrin, d-cis-trans Allethrin, d-trans Allethrin, Bifenthrin, Bioallethrin, Bioallethrin S-cyclopentenyl-isomer, Bioresmethrin, Cycloprothrin, Cyfluthrin, beta-Cyfluthrin, Cyhalothrin, lambda-Cyhalothrin, gamma-Cyhalothrin, Cypermethrin, alpha-Cypermethrin, beta-Cypermethrin, theta-Cypermethrin, zeta-Cypermethrin, Cyphenothrin [(1R)-trans-isomers], Deltamethrin, Empenthrin [(EZ)-(1R)-isomers], Esfenvalerate, Etofenprox, Fenpropathrin, Fenvalerate, Flucythrinate, Flumethrin, tau-Fluvalinate, Halfenprox, Imiprothrin, Kadethrin, Permethrin, Phenothrin [(1R)-trans-isomer], Prallethrin, Pyrethrins, Resmethrin, Silafluofen, Tefluthrin, Tetramethrin, Tetramethrin [(1R)-isomers], Tralomethrin, Transfluthrin, kappa-Bifenthrin, Biopermethrin, Chloroprallethrin, Dimefluthrin, Fenfluthrin, Fenpirithrin, Flufenprox, Heptafluthrin, Meperfluthrin, epsilon-Metofluthrin, Momfluorothrin, epsilon-Momfluorothrin, trans-Permethrin, Profluthrin, Protrifenbute, kappa-Tefluthrin, Terallethrin, Tetramethylfluthrin and Bioethanomethrin.

### (3B) Sodium Channel Modulators (DDT)

DDT and methoxychlor.

(4) Nicotinic Acetylcholine Receptor (nAChR) Competitive Modulators

Acetamiprid, Clothianidin, Dinotefuran, Imidacloprid, Nitenpyram, Thiacloprid, Thiamethoxam, Nicotine, Sulfoxaflor, Flupyradifurone, Triflumezopyrim, Nithiazine, Dicloromezotiaz and Flupyrimin.

(5) Nicotinic Acetylcholine Receptor (nAChR) Allosteric Modulators

Spinetoram and Spinosad.

### (6) Glutamate-Gated Chloride Channel (GluCl) Allosteric Modulators

Abamectin, Emamectin, Emamectin-benzoate, Lepimectin, Milbemectin, Doramectin, Eprinomectin, Ivermectin, Moxidectin and Selamectin.

### (7) Juvenile Hormone Mimics

Hydroprene, Kinoprene, Methoprene; Fenoxycarb; Pyriproxifen, Diofenolan, Epofenonane and Triprene.

### (8) Miscellaneous Non-Specific (Multi-Site) Inhibitors

Methyl bromide, alkyl halides, Chloropicrin, Sodium aluminum fluoride, Sulfuryl fluoride, Borax, Boric acid, Disodium octaborate, Sodium borate, Sodium metaborate, Tartar emetic, Dazomet, Metam, Metam potassium and Metam sodium.

### (9) Chordotonal Organ TRPV Channel Modulators

Pymetrozine, Pyrifluquinazon and Afidopyropen.

### (10) Mite Growth Inhibitors

Clofentezine, Diflovidazin, Hexythiazox and Etoxazole.

### (11) Microbial Disruptors of Insect Midgut Membranes

B.t. subsp. israelensis, B.t. subsp. aizawai, B.t. subsp. kurstaki, B.t. subsp. tenebrionis, B.t. crop proteins: Cry1Ab, Cry1Ac, Cry1Fa, Cry1A.105, Cry2Ab, Vip3A, mCry3A, Cry3Ab, Cry3Bb, Cry34Ab1/Cry35Ab1, and Bacillus sphaericus.

### (12) Inhibitors of Mitochondrial ATP Synthase

Diafenthiuron, Azocyclotin, Cyhexatin, Fenbutatin-oxide, Propargite; Tetradifon.

### (13) Uncouplers of Oxidative Phosphorylation Via Disruption of the Proton Gradient

Chlorfenapyr, DNOC (4,6-dinitro-o-cresol), Sulfluramid, Binapacryl, Dinobuton and Dinocap.

(14) Nicotinic Acetylcholine Receptor (nAChR) Channel Blockers

Bensultap, Cartap hydrochloride, Thiocyclam and Thiosultap-sodium.

### (15) Inhibitors of Chitin Biosynthesis, Type 0

Bistrifluron, Chlorfluazuron, Diflubenzuron, Flucycloxuron, Flufenoxuron, Hexaflumuron, Lufenuron, Novaluron, Noviflumuron, Teflubenzuron, Triflumuron and Fluazuron.

### (16) Inhibitors of Chitin Biosynthesis, Type 1

Buprofezin.

### (17) Moulting Disruptors

Cyromazine.

### (18) Ecdysone Receptor Agonists

Chromafenozide, Halofenozid, Methoxyfenozide and Tebufenozide.

### (19) Octopamine Receptor Agonists

Amitraz and Chlordimeform.

### (20) Mitochondrial Complex III Electron Transport Inhibitors

Hydramethylnon, Acequinocyl, Fluacrypyrim and Bifenazate.

### (21) Mitochondrial Complex I Electron Transport Inhibitors (METI)

Fenazaquin, Fenpyroximate, Pyridaben, Pyrimidifen, Tebufenpyrad, Tolfenpyrad and Rotenone.

### (22) Voltage-Dependent Sodium Channel Blockers

Indoxacarb and Metaflumizone.

### (23) Inhibitors of Acetyl CoA Carboxylase

Spirodiclofen, Spiromesifen, Spirotetramat and Spiropidion.

### (24) Mitochondrial Complex IV Electron Transport Inhibitors

Al-phosphide, Ca-phosphide, Phosphine, Zn-phosphide; Ca-cyanide, Na-cyanide and K-cyanide.

### (25) Mitochondrial Complex II Electron Transport Inhibitors

Cyenopyrafen, Cyflumetofen, Cyetpyrafen; Pyflubumide.

### (28) Ryanodine Receptor Modulators

Chlorantraniliprole, Cyantraniliprole, Cyclaniliprole, Flubendiamide, Cyhalodiamide, Tetrachlorantraniliprole and Tetraniliprole.

### (29) Chordotonal Organ Modulator-Undefined Target Site

Flonicamid.

### (30) GABA-Gated Channel Allosteric Modulators

Broflanilide, Fluxametamide, Isocycloseram; Afoxolaner, Fluralaner, Lotilaner and Sarolaner.

(UN) Azadirachtin, Benzoximate, Bromopropylate, Chinomethionat, Dicofol, Limesulfur, Mancozeb, Pyridalyl, Sulfur, Acynonapyr, Amidoflumet, Benzomate, Benzpyrimoxan, Chlorobenzilate, Dicyclanil, Fenoxacrim, Fentrifanil, Flometoquin, Flubenzimine, Flufenzine, Fluhexafon, Fluopyram, Metoxadiazone, Oxazosulfyl, Tetrasul, Triarathene, Tyclopyrazoflor and Dimpropyridaz.

In one specific embodiment, the insecticidal composition comprises biologically effective doses of the isoxazoline-substituted benzamide compound (e.g., compounds of Formula I, I′ and I″) (ingredient A) and at least one other active ingredient (ingredient B) selected from the following compounds: chlorantraniliprole, spinetoram, lambda-cyhalothrin, emamectin-benzoate, abamectin, indoxacarb, lufenuron, chlorfenapyr, spirotetramat, triflumezopyrim, diafenthiuron, cyetpyrafen, acephate, bifenthrin, methoxyfenozide, spiromesifen, fipronil, spiropidion, buprofezin, pymetrozine, clothianidin, nitenpyram, acetamiprid, afidopyropen, cyantraniliprole, dinotefuran, ethiprole, imidacloprid, sulfoxaflor, thiamethoxam, tolfenpyrad or dimpropyridaz.

In another specific embodiments, the weight ratio of active ingredients A to B in the insecticidal composition is 1:5000~10:1, 1:4000~1:1, 1:3000~1:5, 1:2000~1:10, 1:1000~1:20, 1:800~1:30, 1:600~1:50, 1:500~1:80 or 1:200~1:100.

The term “biologically effective dose” refers to an amount of a biologically active compound (e.g., compounds of Formula I, I′ and I″) sufficient to produce the desired biological effect when applied to (i.e., contacted with) a pest insect to be controlled, or its environment, or a plant, a seed from which the plant has grown, or the location of the plant (e.g., growth medium), thereby protecting the plant from damages caused by the pest insect or achieving other desired effects (e.g., increasing activity of the plant). The compound of the present invention may also be applied prophylactically where pest insects or parasites are expected to appear.

The compound I, its stereoisomers and salts can be converted into agrochemical compositions of conventional types, such as solutions, emulsions, suspensions, dustable powders, powders, pastes, granules, pressings, capsules and mixtures thereof. Examples of composition types are suspensions (e.g., SC, OD, FS), emulsifiable concentrates (e.g., EC), emulsions (e.g., EW, EO, ES, ME), capsules (e.g., CS, ZC), pastes, pastilles, wettable powders or dustable powers (e.g., WP, SP, WS, DP, DS), pressings (e.g., BR, TB, DT), granules (e.g., WG, SG, GR, FG, GG, MG), insecticidal articles (e.g., LN) as well as gel formulations (e.g., GF) for treating plant propagation materials such as seeds.

Suitable auxiliaries are solvents, liquid carriers, solid carriers or fillers, surfactants, dispersants, emulsifiers, wetting agents, adjuvants, solubilizers, penetration enhancers, protective colloids, adhesion agents, thickeners, humectants, repellents, attractants, feeding stimulants, compatibilizers, bactericides, antifreezing agents, defoamers, colorants, tackifiers and binders.

Suitable solvents and liquid carriers are water and organic solvents, such as mineral oil fractions with medium to high boiling points, e.g., kerosene, diesel oil; oils of vegetable or animal origin; aliphatic, cyclic and aromatic hydrocarbons, e.g., toluene, paraffin, tetrahydronaphthalene, alkylated naphthalenes; alcohols, e.g., ethanol, propanol, butanol, benzyl alcohol, cyclohexanol; glycols; DMSO; ketones, e.g., cyclohexanone; esters, e.g., lactate ester, carbonate ester, fatty acid ester, gammabutyrolactone; fatty acids; phosphonates; amines; amides, e.g., N-methylpyrrolidone, fatty acid dimethylamides; and mixtures thereof.

Suitable solid carriers or fillers are mineral earths, e.g., silicate, silica gel, talc, kaolin, limestone, lime, chalk, clay, dolomite, diatomaceous earth, bentonite, calcium sulfate, magnesium sulfate, magnesium oxide; polysaccharides, e.g., cellulose, starch; fertilizers, e.g., ammonium sulfate, ammonium phosphate, ammonium nitrate, ureas; products of vegetable origin, e.g., cereal meal, tree bark meal, wood meal, nutshell meal, and mixtures thereof.

Suitable surfactants are surface active compounds, such as anionic, cationic, nonionic and amphoteric surfactants, block polymers, polyelectrolytes, and mixtures thereof. Such surfactants can be used as emulsifiers, dispersants, solubilizers, wetting agents, penetration enhancers, protective colloids, or adjuvants.

Suitable anionic surfactants are alkali metal, alkaline earth metal or ammonium salts of sulfonates, sulfates, phosphates, carboxylates, and mixtures thereof. Examples of sulfonates are alkylarylsulfonates, diphenylsulfonates, alpha-olefin sulfonates, lignin sulfonates, sulfonates of fatty acids and oils, sulfonates of ethoxylated alkylphenols, sulfonates of alkoxylated arylphenols, sulfonates of condensed naphthalenes, sulfonates of dodecyl- and tridecylbenzenes, sulfonates of naphthalenes and alkylnaphthalenes, sulfosuccinates or sulfosuccinamates. Examples of sulfates are sulfates of fatty acids and oils, sulfates of ethoxylated alkylphenols, sulfates of alcohols, sulfates of ethoxylated alcohols, or sulfates of fatty acid esters. Examples of phosphates are phosphate esters. Examples of carboxylates are alkyl carboxylates, and carboxylated alcohols or alkylphenol ethoxylates.

Suitable nonionic surfactants are alkoxylates, N-substituted fatty acid amides, amine oxides, esters, sugar-based surfactants, polymeric surfactants, and mixtures thereof. Examples of alkoxylates are compounds such as alcohols, alkylphenols, amines, amides, arylphenols, fatty acids or fatty acid esters which have been alkoxylated with 1 to 50 equivalents. Ethylene oxide and/or propylene oxide may be employed for the alkoxylation, preferably ethylene oxide. Examples of N-substituted fatty acid amides are fatty acid glucamides or fatty acid alkanolamides. Examples of esters are fatty acid esters, glycerol esters or monoglycerides. Examples of sugar-based surfactants are sorbitans, ethoxylated sorbitans, sucrose and glucose esters or alkylpolyglucosides. Examples of polymeric surfactants are homo- or copolymers of vinylpyrrolidones, vinyl alcohols, or vinyl acetates.

Suitable cationic surfactants are quaternary surfactants, for example, a quaternary ammonium compound with one or two hydrophobic groups, or salts of a long chain primary amine. Suitable amphoteric surfactants are alkylbetaines and imidazolines. Suitable block polymers are block polymers of the A-B or A-B-A type comprising blocks of polyethylene oxide and polypropylene oxide, or block polymers of the A-B-C type comprising alkanol, polyethylene oxide and polypropylene oxide. Suitable polyelectrolytes are polyacids or polybases. Examples of polyacids are alkali metal salts of polyacrylic acid or polyacid comb polymers. Examples of polybases are polyvinylamines or polyethyleneamines.

Suitable adjuvants are compounds which have neglectable or even no pesticidal activity themselves and improve the biological performance of the compound I on the target. Examples are surfactants, mineral or vegetable oils, and other auxiliaries.

Suitable thickeners are polysaccharides (e.g., xanthan gum, carboxymethylcellulose), inorganic clays (organically modified or unmodified), polycarboxylates, and silicates.

Suitable bactericides are bronopols and isothiazolinone derivatives such as alkylisothiazolinones and benzisothiazolinones.

Suitable antifreezing agents are ethylene glycol, propylene glycol, urea and glycerin.

Suitable defoamers are polysiloxanes, long chain alcohols, and salts of fatty acids.

Suitable colorants (e.g., in red, blue, or green) are pigments of low water solubility and water-soluble dyes. Examples are inorganic colorants (e.g., iron oxide, titanium oxide, iron hexacyanoferrate) and organic colorants (e.g., alizarin-, azo- and phthalocyanine colorants).

Suitable tackifiers or binders are polyvinylpyrrolidons, polyvinylacetates, polyvinyl alcohols, polyacrylates, biological or synthetic waxes, and cellulose ethers.

The present invention also provides a method for controlling pest insects, which comprises exposing a pest insect or its environment to a biologically effective dose of the isoxazoline-substituted benzamide compound, its stereoisomer and agriculturally or veterinary acceptable salt or the composition.

In one specific embodiment, a biologically effective dose of the compound of the present invention or the composition as defined above is used for treating pest insects, their food sources, habitats or breeding places; or cultivated plants, plant propagation materials (e.g., seeds), soil, areas, materials or environment where pest insects grow or are likely to grow; or materials, cultivated plants, plant propagation materials (e.g., seeds), soil, surfaces or spaces to be protected against attack or infestation by pest insects.

The present invention also relates to a use of the compound of the present invention, a stereoisomer and/or an agriculturally or veterinary acceptable salt thereof in pest control.

The term “pest control” refers to inhibiting the development of pest insects (including death, decreased food intake, and/or mating disruption), and related expressions can be defined similarly.

The term “plant propagation material” is to be understood to denote all generative parts of a plant such as seeds, as well as vegetative plant materials that can be used for propagating plants, such as cuttings and tubers (e.g., potatoes). These include seeds, roots, fruits, tubers, corms, underground stems, shoots, sprouts and other plant parts, and seedlings and young plants transplanted from soil after germination or emergence are also included. A plant propagation material may be treated prophylactically with plant protection compounds at or prior to planting or transplanting. The young plant may also be protected by complete or partial treatment by dipping or watering prior to transplanting.

The term “plant” includes any type of plant, including “non-cultivated plants” and especially “cultivated plants”.

The term “non-cultivated plant” refers to any wild-type variety or relevant variety or relevant genus of a cultivated plant.

The term “cultivated plant” is to be understood to include plants which have been modified by breeding, mutagenesis or genetic engineering, including but not limited to agricultural biotechnology products that are commercially available or developed (see http://www.bio.org/speeches/pubs/er/agri_products.asp). A genetically modified plant is a plant whose genetic material has been modified using recombinant DNA techniques that are not easily obtained by crossing, mutation or natural recombination under natural conditions. Normally one or more genes are integrated into the genetic material of a genetically modified plant to improve certain performances of the plant. Such genetic modifications also include, but are not limited to, targeted post-translational modification of proteins, oligopeptides or polypeptides, for example, by glycosylation or polymer additions such as prenylated, acetylated or farnesylated moieties or PEG moieties.

### DETAILED EMBODIMENTS OF THE INVENTION

The following examples are used to illustrate the present invention and should not be regarded as limiting the present invention in any way. The scope of rights claimed by the present invention is described by the claims.

In view of the economy and diversity of the compounds, some compounds were preferably synthesized. Among the many synthesized compounds, selected ones are listed in Table 1 below. The specific compound structure and corresponding compound information are as set forth in Table 1. The compounds in Table 1 are only to better illustrate the present invention, but do not limit the present invention. For those skilled in the art, it should not be understood that the scope of the above-mentioned subject matters of the present invention is limited to the following compounds.

TABLE 1
Compound structure and ¹H NMR thereof

| | |
|---|---|
| | I |
| 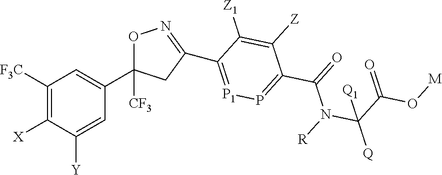 | |

| No. | Q | M | R | P | X | Y | Z | Z₁ | Q₁ | P₁ | ¹H NMR |
|---|---|---|---|---|---|---|---|---|---|---|---|
| 1 | CH₃ | CH₃ | H | CH | H | Cl | CH₃ | H | H | CH | ¹H NMR (300 MHz, Chloroform-d) δ |
| | | | | | | | | | | | 7.82 (s, 1H), 7.76 (s, 1H), 7.68 (s, |
| | | | | | | | | | | | 1H), 7.55-7.43 (m, 3H), 6.39 (d, J = |
| | | | | | | | | | | | 7.5 Hz, 1H), 4.83-4.71 (m, 1H), |
| | | | | | | | | | | | 4.10 (d, J = 17.4 Hz, 1H), 3.79 (s, |
| | | | | | | | | | | | 3H), 3.73 (d, J = 17.4 Hz, 1H), 2.47 |
| | | | | | | | | | | | (s, 3H), 1.52 (d, J = 7.2 Hz, 3H). |
| 2 | CH₃ | CH₃ | H | CH | F | Cl | CH₃ | H | H | CH | ¹H NMR (300 MHz, DMSO-d₆) δ |
| | | | | | | | | | | | 8.77 (d, J = 7.2 Hz, 1H), 8.17-8.16 |
| | | | | | | | | | | | (m, 1H), 7.88-7.86 (m, 1H), 7.60- |
| | | | | | | | | | | | 7.58 (m, 2H), 7.42 (d, J = 8.1 Hz, |
| | | | | | | | | | | | 1H), 4.45-4.38 (m, 3H), 3.65 (s, |
| | | | | | | | | | | | 3H), 2.36 (s, 3H), 1.35 (d, J = 7.2 |
| | | | | | | | | | | | Hz, 3H). |
| 3 | CH₃ | CH₃ | H | CH | Cl | Cl | CH₃ | H | H | CH | ¹H NMR (300 MHz, Chloroform-d) δ |
| | | | | | | | | | | | 7.94 (d, J = 2.1 Hz, 1H), 7.84 (d, J = |
| | | | | | | | | | | | 2.1 Hz, 1H), 7.52-7.47 (m, 2H), |
| | | | | | | | | | | | 7.43 (d, J = 8.4 Hz, 1H), 6.35(d, J = |
| | | | | | | | | | | | 7.5 Hz, 1H), 4.83-4.73 (m, 1H), |
| | | | | | | | | | | | 4.13 (d, J = 17.1 Hz, 1H), 3.8 (s, |
| | | | | | | | | | | | 3H), 3.69 (d, J = 17.1 Hz, 1H), 2.48 |
| | | | | | | | | | | | (s, 3H), 1.53 (d, J = 7.2 Hz, 3H). |
| 4 | CH₃ | CH₃ | CH₃ | CH | Cl | Cl | CH₃ | H | H | CH | ¹H NMR (300 MHz, Chloroform-d) δ |
| | | | | | | | | | | | 7.95 (d, J = 2.1 Hz, 1H), 7.84 (d, J = |
| | | | | | | | | | | | 2.1 Hz, 1H), 7.53-7.49 (m, 2H), |
| | | | | | | | | | | | 7.46 (d, J = 8.4 Hz, 1H), 5.40-5.34 |
| | | | | | | | | | | | (m, 1H), 4.17-4.09 (m, 1H), 3.80- |
| | | | | | | | | | | | 3.65 (m, 4H), 3.01 (d, J = 6.6 Hz, |
| | | | | | | | | | | | 1H), 2.75 (s, 2H), 2.37 (s, 3H), 1.53 |
| | | | | | | | | | | | (d, J = 7.5 Hz, 2H), 1.42-1.38 (m, |
| | | | | | | | | | | | 1H). |
| 5 | CH₃ | CH₃ | CH₂CH₃ | CH | Cl | Cl | CH₃ | H | H | CH | |
| 6 | CH₃ | CH₃ | 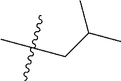 [SMILES: *CC(C)C] | CH | Cl | Cl | CH₃ | H | H | CH | |
| 7 | CH₃ | CH₃ | 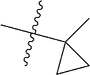 [SMILES: *C1(C)CC1] | CH | Cl | Cl | CH₃ | H | H | CH | |
| 8 | CH₃ | CH₃ | 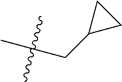 [SMILES: *CC1CC1] | CH | Cl | Cl | CH₃ | H | H | CH | |
| 9 | CH₃ | CH₃ | CH₂CF₃ | CH | Cl | Cl | CH₃ | H | H | CH | |
| 10 | CH₃ | CH₃ | OH | CH | Cl | Cl | CH₃ | H | H | CH | |
| 11 | CH₃ | CH₃ | OCH₃ | CH | Cl | Cl | CH₃ | H | H | CH | |
| 12 | CH₃ | CH₃ | 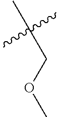 [SMILES: *COC] | CH | Cl | Cl | CH₃ | H | H | CH | |
| 13 | CH₃ | CH₃ | 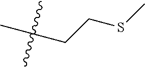 [SMILES: *CCSC] | CH | Cl | Cl | CH₃ | H | H | CH | |
| 14 | CH₃ | CH₃ | 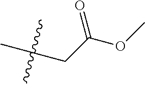 [SMILES: *CC(=O)OC] | CH | Cl | Cl | CH₃ | H | H | CH | |
| 15 | CH₃ | CH₃ | 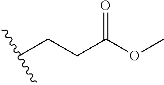 [SMILES: *CCC(=O)OC] | CH | Cl | Cl | CH₃ | H | H | CH | |
| 16 | CH₃ | CH₃ | 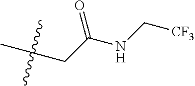 [SMILES: *CC(=O)NCC(F)(F)F] | CH | Cl | Cl | CH₃ | H | H | CH | |
| 17 | CH₃ | CH₃ | 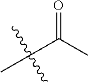 [SMILES: *C(C)=O] | CH | Cl | Cl | CH₃ | H | H | CH | |
| 18 | CH₃ | CH₃ | 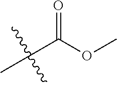 [SMILES: *C(=O)OC] | CH | Cl | Cl | CH₃ | H | H | CH | |
| 19 | CH₃ | CH₃ | 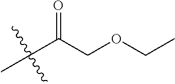 [SMILES: *C(=O)COCC] | CH | Cl | Cl | CH₃ | H | H | CH | |
| 20 | CH₃ | CH₃ | 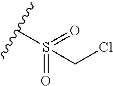 [SMILES: *S(=O)(=O)CCl] | CH | Cl | Cl | CH₃ | H | H | CH | |
| 21 | CH₃ | CH₃ | 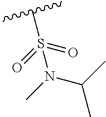 [SMILES: *S(=O)(=O)N(C)C(C)C] | CH | Cl | Cl | CH₃ | H | H | CH | |
| 22 | CH₃ | CH₃ | 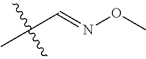 [SMILES: */C=N/OC] | CH | Cl | Cl | CH₃ | H | H | CH | |
| 23 | CH₃ | CH₃ | 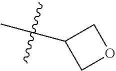 [SMILES: *C1COC1] | CH | Cl | Cl | CH₃ | H | H | CH | |
| 24 | CH₃ | CH₃ | 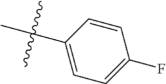 [SMILES: *c1ccc(F)cc1] | CH | Cl | Cl | CH₃ | H | H | CH | |
| 25 | CH₃ | CH₃ | 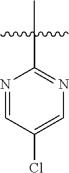 [SMILES: *c1ncc(Cl)cn1] | CH | Cl | Cl | CH₃ | H | H | CH | |
| 26 | CH₃ | CH₃ | 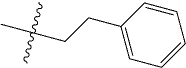 [SMILES: *CCc1ccccc1] | CH | Cl | Cl | CH₃ | H | H | CH | |
| 27 | CH₃ | CH₃ | 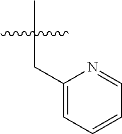 [SMILES: *Cc1ccccn1] | CH | Cl | Cl | CH₃ | H | H | CH | |
| 28 | CH₃ | CH₃ | 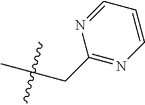 [SMILES: *Cc1ncccn1] | CH | Cl | Cl | CH₃ | H | H | CH | |
| 29 | CH₃ | CH₃ | 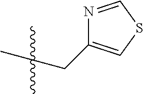 [SMILES: *Cc1cscn1] | CH | Cl | Cl | CH₃ | H | H | CH | |
| 30 | CH₃ | CH₂CH₃ | H | CH | Cl | Cl | CH₃ | H | H | CH | ¹H NMR (300 MHz, Chloroform-d) δ |
| | | | | | | | | | | | 7.94 (d, J = 2.1 Hz, 1H), 7.84 (d, J = |
| | | | | | | | | | | | 2.1 Hz, 1H), 7.52-7.47 (m, 2H), |
| | | | | | | | | | | | 7.43 (d, J = 8.4 Hz, 1H), 6.37 (d, J = |
| | | | | | | | | | | | 7.2 Hz, 1H), 4.80-4.70 (m, 1H), |
| | | | | | | | | | | | 4.25(q, J = 7.2 Hz, 2H), 4.13 (d, J = |
| | | | | | | | | | | | 17.1 Hz, 1H), 3.69 (d, J = 17.1 Hz, |
| | | | | | | | | | | | 1H), 2.48 (s, 3H), 1.53 (d, J = 7.2 |
| | | | | | | | | | | | Hz, 3H), 1.31 (t, J = 7.2 Hz, 3H). |
| 31 | CH₃ | 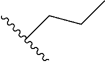 [SMILES: *CCC] | H | CH | Cl | Cl | CH₃ | H | H | CH | ¹H NMR (300 MHz, Chloroform-d) δ 7.94 (d, J = 2.1 Hz, 1H), 7.84 (d, J = 2.1 Hz, 1H), 7.52-7.47 (m, 2H), 7.43 (d, J = 8.4 Hz, 1H), 6.40 (d, J = |
| | | | | | | | | | | | 7.5 Hz, 1H), 4.83-4.73 (m, 1H), |
| | | | | | | | | | | | 4.17-4.11 (m, 3H), 3.71 (d, J = 17.4 |
| | | | | | | | | | | | Hz, 1H), 2.47 (s, 3H), 1.74-1.64 (m, |
| | | | | | | | | | | | 2H), 1.53 (d, J = 7.2 Hz, 3H), 0.96 |
| | | | | | | | | | | | (t, J = 7.5 Hz, 3H). |
| 32 | CH₃ | 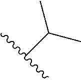 [SMILES: *C(C)C] | H | CH | Cl | Cl | CH₃ | H | H | CH | ¹H NMR (300 MHz, Chloroform-d) δ 7.95 (d, J = 2.1 Hz, 1H), 7.84 (d, J = 2.1 Hz, 1H), 7.53-7.49 (m, 2H), 7.46 (d, J = 8.4 Hz, 1H), 6.36 (d, J = 7.5 Hz, 1H), 5.13-5.04 (m, 1H), |
| | | | | | | | | | | | 4.75-4.67 (m, 1H), 4.14 (d, J = 17.1 |
| | | | | | | | | | | | Hz, 1H), 3.70 (d, J = 17.1 Hz, 1H), |
| | | | | | | | | | | | 2.49 (s, 3H), 1.51 (d, J = 7.2 Hz, |
| | | | | | | | | | | | 3H), 1.31-1.26 (m, 6H). |
| 33 | CH₃ | 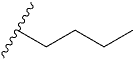 [SMILES: *CCCC] | H | CH | Cl | Cl | CH₃ | H | H | CH | |
| 34 | CH₃ | 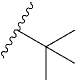 [SMILES: *C(C)(C)C] | H | CH | Cl | Cl | CH₃ | H | H | CH | |
| 35 | CH₃ | 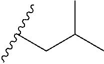 [SMILES: *CC(C)C] | H | CH | Cl | Cl | CH₃ | H | H | CH | |
| 36 | CH₃ | 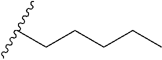 [SMILES: *CCCCC] | H | CH | Cl | Cl | CH₃ | H | H | CH | |
| 37 | CH₃ | 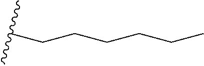 [SMILES: *CCCCCC] | H | CH | Cl | Cl | CH₃ | H | H | CH | |
| 38 | CH₃ | 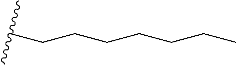 [SMILES: *CCCCCCC] | H | CH | Cl | Cl | CH₃ | H | H | CH | |
| 39 | CH₃ | 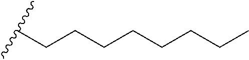 [SMILES: *CCCCCCCC] | H | CH | Cl | Cl | CH₃ | H | H | CH | |
| 40 | CH₃ | 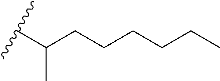 [SMILES: *C(C)CCCCCC] | H | CH | Cl | Cl | CH₃ | H | H | CH | |
| 41 | CH₃ | 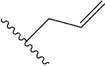 [SMILES: *CC=C] | H | CH | Cl | Cl | CH₃ | H | H | CH | ¹H NMR (300 MHz, Chloroform-d) δ 7.94 (d, J = 2.1 Hz, 1H), 7.84 (d, J = 2.1 Hz, 1H), 7.52-7.47 (m, 2H), 7.43 (d, J = 8.4 Hz, 1H), 6.40 (d, J = |
| | | | | | | | | | | | 7.5 Hz, 1H), 6.00-5.87 (m, 1H), |
| | | | | | | | | | | | 5.39-5.27 (m, 2H), 4.83-4.73 (m, |
| | | | | | | | | | | | 1H), 4.70-4.67 (m, 2H), 4.14 (d, J = |
| | | | | | | | | | | | 17.4 Hz, 1H), 3.71 (d, J = 17.4 Hz, |
| | | | | | | | | | | | 1H), 2.47 (s, 3H), 1.53 (d, J = 7.2 |
| | | | | | | | | | | | Hz, 3H). |
| 42 | CH₃ | 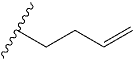 [SMILES: *CCC=C] | H | CH | Cl | Cl | CH₃ | H | H | CH | |
| 43 | CH₃ | 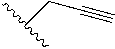 [SMILES: *CC#C] | H | CH | Cl | Cl | CH₃ | H | H | CH | ¹H NMR (300 MHz, Chloroform-d) δ 7.94 (d, J = 2.1 Hz, 1H), 7.84 (d, J = 2.1 Hz, 1H), 7.52-7.47 (m, 2H), 7.43 (d, J = 8.4 Hz, 1H), 6.33 (d, J = |
| | | | | | | | | | | | 7.5 Hz, 1H), 4.86-4.76 (m, 3H), |
| | | | | | | | | | | | 4.13 (d, J = 17.1 Hz, 1H), 3.69 (d, |
| | | | | | | | | | | | J = 17.1 Hz, 1H), 2.53-2.51 (m, 1H), |
| | | | | | | | | | | | 2.48 (s, 3H), 1.53 (d, J = 7.2 Hz, |
| | | | | | | | | | | | 3H). |
| 44 | CH₃ | 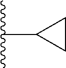 [SMILES: *C1CC1] | H | CH | Cl | Cl | CH₃ | H | H | CH | ¹H NMR (300 MHz, Chloroform-d) δ 7.95 (d, J = 2.1 Hz, 1H), 7.84 (d, J = 2.1 Hz, 1H), 7.55-7.43 (m, 3H), 6.35 (d, J = 7.5 Hz, 1H), 4.79-4.63 (m, 1H), 4.27-4.10 (m, 2H), 3.71 (d, |
| | | | | | | | | | | | J = 17.1 Hz, 1H), 2.47 (s, 3H), 1.49 |
| | | | | | | | | | | | (d, J = 7.2 Hz, 3H), 0.83-0.68 (m, |
| | | | | | | | | | | | 4H). |
| 45 | CH₃ | 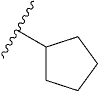 [SMILES: *C1CCCC1] | H | CH | Cl | Cl | CH₃ | H | H | CH | |
| 46 | CH₃ | 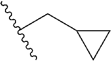 [SMILES: *CC1CC1] | H | CH | Cl | Cl | CH₃ | H | H | CH | |
| 47 | CH₃ | 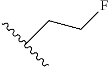 [SMILES: *CCF] | H | CH | Cl | Cl | CH₃ | H | H | CH | |
| 48 | CH₃ | 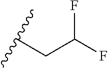 [SMILES: *CC(F)F] | H | CH | Cl | Cl | CH₃ | H | H | CH | |
| 49 | CH₃ | CH₂CF₃ | H | CH | Cl | Cl | CH₃ | H | H | CH | |
| 50 | CH₃ | 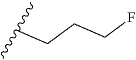 [SMILES: *CCCF] | H | CH | Cl | Cl | CH₃ | H | H | CH | |
| 51 | CH₃ | 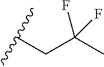 [SMILES: *CC(C)(F)F] | H | CH | Cl | Cl | CH₃ | H | H | CH | |
| 52 | CH₃ | 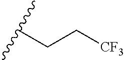 [SMILES: *CCC(F)(F)F] | H | CH | Cl | Cl | CH₃ | H | H | CH | |
| 53 | CH₃ | 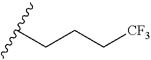 [SMILES: *CCCC(F)(F)F] | H | CH | Cl | Cl | CH₃ | H | H | CH | |
| 54 | CH₃ | 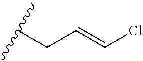 [SMILES: *C/C=C/Cl] | H | CH | Cl | Cl | CH₃ | H | H | CH | |
| 55 | CH₃ | 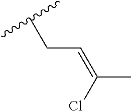 [SMILES: *C/C=C(/C)Cl] | H | CH | Cl | Cl | CH₃ | H | H | CH | |
| 56 | CH₃ | 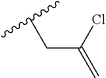 [SMILES: *CC(=C)Cl] | H | CH | Cl | Cl | CH₃ | H | H | CH | |
| 57 | CH₃ | 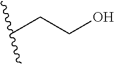 [SMILES: *CCO] | H | CH | Cl | Cl | CH₃ | H | H | CH | ¹H NMR (300 MHz, Chloroform-d) δ 7.94 (d, J = 2.1 Hz, 1H), 7.84 (d, J = 2.1 Hz, 1H), 7.52-7.47 (m, 2H), 7.43 (d, J = 8.4 Hz, 1H), 6.38 (d, J = 7.2 Hz, 1H), 4.81-4.67 (m, 1H), |
| | | | | | | | | | | | 4.42-4.24 (m, 2H), 4.13 (d, J = 17.1 |
| | | | | | | | | | | | Hz, 1H), 3.93-3.81 (m, 2H), 3.71 (d, |
| | | | | | | | | | | | J = 17.1 Hz, 1H), 2.48 (s, 3H), 1.55 |
| | | | | | | | | | | | (d, J = 7.2 Hz, 3H). |
| 58 | CH₃ | 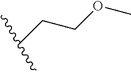 [SMILES: *CCOC] | H | CH | Cl | Cl | CH₃ | H | H | CH | |
| 59 | CH₃ | 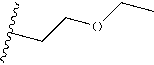 [SMILES: *CCOCC] | H | CH | Cl | Cl | CH₃ | H | H | CH | |
| 60 | CH₃ | 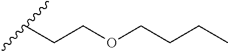 [SMILES: *CCOCCCC] | H | CH | Cl | Cl | CH₃ | H | H | CH | |
| 61 | CH₃ | 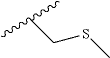 [SMILES: *CSC] | H | CH | Cl | Cl | CH₃ | H | H | CH | |
| 62 | CH₃ | 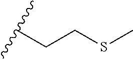 [SMILES: *CCSC] | H | CH | Cl | Cl | CH₃ | H | H | CH | |
| 63 | CH₃ | 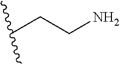 [SMILES: *CCN] | H | CH | Cl | Cl | CH₃ | H | H | CH | |
| 64 | CH₃ | 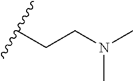 [SMILES: *CCN(C)C] | H | CH | Cl | Cl | CH₃ | H | H | CH | |
| 65 | CH₃ | 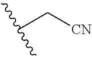 [SMILES: *CC#N] | H | CH | Cl | Cl | CH₃ | H | H | CH | ¹H NMR (300 MHz, Chloroform-d) δ 7.94 (d, J = 2.1 Hz, 1H), 7.83 (d, J = 2.1 Hz, 1H), 7.56-7.48 (m, 2H), 7.46-7.40 (m, 1H), 6.32 (d, J = 7.5 |
| | | | | | | | | | | | Hz, 1H), 4.94-4.71 (m, 3H), 4.17 (d, |
| | | | | | | | | | | | J = 17.1 Hz, 1H), 3.71 (d, J = 17.1 |
| | | | | | | | | | | | Hz, 1H), 2.46 (s, 3H), 1.57 (d, J = |
| | | | | | | | | | | | 7.2 Hz, 3H). |
| 66 | CH₃ | 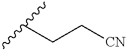 [SMILES: *CCC#N] | H | CH | Cl | Cl | CH₃ | H | H | CH | |
| 67 | CH₃ | 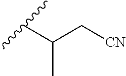 [SMILES: *C(C)CC#N] | H | CH | Cl | Cl | CH₃ | H | H | CH | |
| 68 | CH₃ | 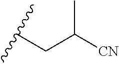 [SMILES: *CC(C)C#N] | H | CH | Cl | Cl | CH₃ | H | H | CH | |
| 69 | CH₃ | 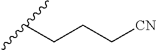 [SMILES: *CCCC#N] | H | CH | Cl | Cl | CH₃ | H | H | CH | |
| 70 | CH₃ | 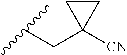 [SMILES: *CC1(C#N)CC1] | H | CH | Cl | Cl | CH₃ | H | H | CH | |
| 71 | CH₃ | 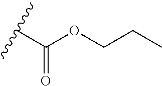 [SMILES: *C(=O)OCCC] | H | CH | Cl | Cl | CH₃ | H | H | CH | |
| 72 | CH₃ | 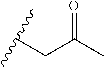 [SMILES: *CC(C)=O] | H | CH | Cl | Cl | CH₃ | H | H | CH | |
| 73 | CH₃ | 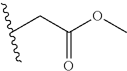 [SMILES: *CC(=O)OC] | H | CH | Cl | Cl | CH₃ | H | H | CH | ¹H NMR (300 MHz, Chloroform-d) δ 7.94 (d, J = 2.1 Hz, 1H), 7.83 (d, J = 2.1 Hz, 1H), 7.53-7.42 (m, 3H), 6.39 (d, J = 7.5 Hz, 1H), 4.93-4.83 (m, 1H), 4.12-4.04 (m, 2H), 4.16 (d, |
| | | | | | | | | | | | J = 17.1 Hz, 1H), 3.77 (s, 3H), 3.71 |
| | | | | | | | | | | | (d, J = 17.1 Hz, 1H), 2.46 (s, 3H), |
| | | | | | | | | | | | 1.60 (d, J = 7.2 Hz, 3H). |
| 74 | CH₃ | 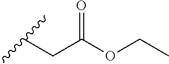 [SMILES: *CC(=O)OCC] | H | CH | Cl | Cl | CH₃ | H | H | CH | |
| 75 | CH₃ | 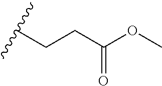 [SMILES: *CCC(=O)OC] | H | CH | Cl | Cl | CH₃ | H | H | CH | |
| 76 | CH₃ | 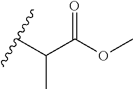 [SMILES: *C(C)C(=O)OC] | H | CH | Cl | Cl | CH₃ | H | H | CH | |
| 77 | CH₃ | 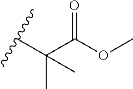 [SMILES: *C(C)(C)C(=O)OC] | H | CH | Cl | Cl | CH₃ | H | H | CH | |
| 78 | CH₃ | 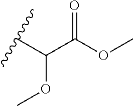 [SMILES: *C(OC)C(=O)OC] | H | CH | Cl | Cl | CH₃ | H | H | CH | |
| 79 | CH₃ | 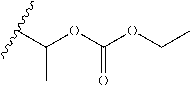 [SMILES: *C(C)OC(=O)OCC] | H | CH | Cl | Cl | CH₃ | H | H | CH | |
| 80 | CH₃ | 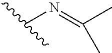 [SMILES: *N=C(C)C] | H | CH | Cl | Cl | CH₃ | H | H | CH | ¹H NMR (300 MHz, Chloroform-d) δ 7.95 (d, J = 1.8 Hz, 1H), 7.84 (d, J = 1.8 Hz, 1H), 7.55-7.44 (m, 3H), 6.42 (d, J = 7.5 Hz, 1H), 4.98-4.90 |
| | | | | | | | | | | | (m, 1H), 4.14 (d, J = 17.4 Hz, 1H), |
| | | | | | | | | | | | 3.71 (d, J = 17.4 Hz, 1H), 2.48 (s, |
| | | | | | | | | | | | 3H), 2.09 (s, 3H), 2.06 (s, 3H), 1.59 |
| | | | | | | | | | | | (d, J = 7.2 Hz, 3H). |
| 81 | CH₃ | 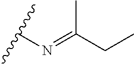 [SMILES: */N=C(\C)CC] | H | CH | Cl | Cl | CH₃ | H | H | CH | |
| 82 | CH₃ | 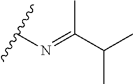 [SMILES: */N=C(\C)C(C)C] | H | CH | Cl | Cl | CH₃ | H | H | CH | |
| 83 | CH₃ | 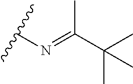 [SMILES: */N=C(\C)C(C)(C)C] | H | CH | Cl | Cl | CH₃ | H | H | CH | |
| 84 | CH₃ | 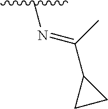 [SMILES: */N=C(\C)C1CC1] | H | CH | Cl | Cl | CH₃ | H | H | CH | |
| 85 | CH₃ | 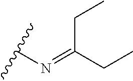 [SMILES: *N=C(CC)CC] | H | CH | Cl | Cl | CH₃ | H | H | CH | |
| 86 | CH₃ | 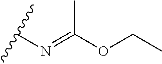 [SMILES: */N=C(\C)OCC] | H | CH | Cl | Cl | CH₃ | H | H | CH | |
| 87 | CH₃ | 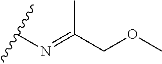 [SMILES: */N=C(\C)COC] | H | CH | Cl | Cl | CH₃ | H | H | CH | |
| 88 | CH₃ | 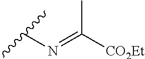 [SMILES: */N=C(\C)C(=O)OCC] | H | CH | Cl | Cl | CH₃ | H | H | CH | |
| 89 | CH₃ | 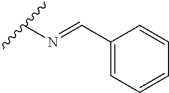 [SMILES: */N=C/c1ccccc1] | H | CH | Cl | Cl | CH₃ | H | H | CH | |
| 90 | CH₃ | 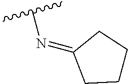 [SMILES: *N=C1CCCC1] | H | CH | Cl | Cl | CH₃ | H | H | CH | |
| 91 | CH₃ | 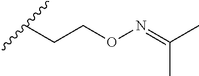 [SMILES: *CCON=C(C)C] | H | CH | Cl | Cl | CH₃ | H | H | CH | |
| 92 | CH₃ | 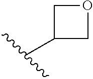 [SMILES: *C1COC1] | H | CH | Cl | Cl | CH₃ | H | H | CH | |
| 93 | CH₃ | 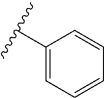 [SMILES: *c1ccccc1] | H | CH | Cl | Cl | CH₃ | H | H | CH | |
| 94 | CH₃ | 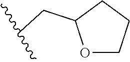 [SMILES: *CC1CCCO1] | H | CH | Cl | Cl | CH₃ | H | H | CH | |
| 95 | CH₃ | 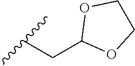 [SMILES: *CC1OCCO1] | H | CH | Cl | Cl | CH₃ | H | H | CH | |
| 96 | CH₃ | 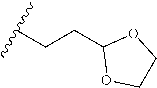 [SMILES: *CCC1OCCO1] | H | CH | Cl | Cl | CH₃ | H | H | CH | |
| 97 | CH₃ | 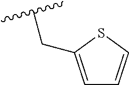 [SMILES: *Cc1cccs1] | H | CH | Cl | Cl | CH₃ | H | H | CH | |
| 98 | CH₃ | 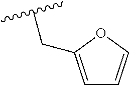 [SMILES: *Cc1ccco1] | H | CH | Cl | Cl | CH₃ | H | H | CH | |
| 99 | CH₃ | 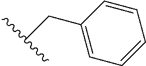 [SMILES: *Cc1ccccc1] | H | CH | Cl | Cl | CH₃ | H | H | CH | ¹H NMR (300 MHz, Chloroform-d) δ 7.94 (d, J = 2.1 Hz, 1H), 7.84 (d, J = 2.1 Hz, 1H), 7.52-7.47 (m, 2H), 7.43 (d, J = 8.4 Hz, 1H), 7.36-7.34 (m, 5H), 6.35 (d, J = 7.5 Hz, 1H), |
| | | | | | | | | | | | 5.22 (d, J = 4.5 Hz, 2H), 4.81-4.67 |
| | | | | | | | | | | | (m, 1H), 4.13 (d, J = 17.1 Hz, 1H), |
| | | | | | | | | | | | 3.71 (d, J = 17.1 Hz, 1H), 2.46 (s, |
| | | | | | | | | | | | 3H), 1.54 (d, J = 7.2 Hz, 3H). |
| 100 | CH₃ | 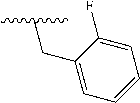 [SMILES: *Cc1ccccc1F] | H | CH | Cl | Cl | CH₃ | H | H | CH | |
| 101 | CH₃ | 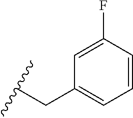 [SMILES: *Cc1cccc(F)c1] | H | CH | Cl | Cl | CH₃ | H | H | CH | |
| 102 | CH₃ | 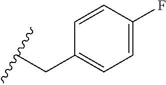 [SMILES: *Cc1ccc(F)cc1] | H | CH | Cl | Cl | CH₃ | H | H | CH | |
| 103 | CH₃ | 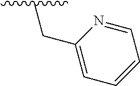 [SMILES: *Cc1ccccn1] | H | CH | Cl | Cl | CH₃ | H | H | CH | |
| 104 | CH₃ | 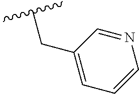 [SMILES: *Cc1cccnc1] | H | CH | Cl | Cl | CH₃ | H | H | CH | |
| 105 | CH₃ | 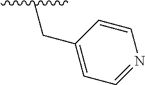 [SMILES: *Cc1ccncc1] | H | CH | Cl | Cl | CH₃ | H | H | CH | |
| 106 | CH₃ | 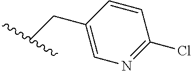 [SMILES: *Cc1ccc(Cl)nc1] | H | CH | Cl | Cl | CH₃ | H | H | CH | ¹H NMR (300 MHz, Chloroform-d) δ 8.40 (d, J = 2.4 Hz, 1H), 7.94 (d, J = 2.1 Hz, 1H), 7.83 (d, J = 2.1 Hz, 1H), 7.73-7.65 (m, 1H), 7.55-7.48 (m, 2H), 7.43 (d, J = 8.4 Hz, 1H), |
| | | | | | | | | | | | 7.35 (d, J = 8.4 Hz, 1H), 6.33 (d, J = |
| | | | | | | | | | | | 7.5 Hz, 1H), 5.20 (d, J = 2.1 Hz, |
| | | | | | | | | | | | 2H), 4.80-4.76 (m, 1H), 4.13 (d, J = |
| | | | | | | | | | | | 17.1 Hz, 1H), 3.71 (d, J = 17.1 Hz, |
| | | | | | | | | | | | 1H), 2.44 (s, 3H), 1.51 (d, J = 7.2 |
| | | | | | | | | | | | Hz, 3H). |
| 107 | CH₃ | 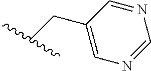 [SMILES: *Cc1cncnc1] | H | CH | Cl | Cl | CH₃ | H | H | CH | ¹H NMR (300 MHz, Chloroform-d) δ 9.22 (s, 1H), 8.79 (s, 2H), δ 7.94 (d, J = 2.1 Hz, 1H), 7.84 (d, J = 2.1 Hz, 1H), 7.52-7.47 (m, 2H), 7.43 (d, J = 8.4 Hz, 1H), 6.26 (d, J = 7.5 Hz, |
| | | | | | | | | | | | 1H), 5.25 (d, J = 4.2 Hz, 2H), 4.83- |
| | | | | | | | | | | | 4.73 (m, 1H), 4.14 (d, J = 17.4 Hz, |
| | | | | | | | | | | | 1H), 3.71 (d, J = 17.4 Hz, 1H), 2.47 |
| | | | | | | | | | | | (s, 3H), 1.53 (d, J = 7.2 Hz, 3H). |
| 108 | CH₃ | 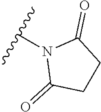 [SMILES: *N1C(=O)CCC1=O] | H | CH | Cl | Cl | CH₃ | H | H | CH | |
| 109 | CH₂CH₃ | CH₃ | H | CH | Cl | Cl | CH₃ | H | H | CH | ¹H NMR (300 MHz, CDCl₃) δ 7.95 |
| | | | | | | | | | | | (d, J = 2.1 Hz, 1H), 7.84 (d, J = 2.1 |
| | | | | | | | | | | | Hz, 1H), 7.55-7.45 (m, 3H), 6.30 (d, |
| | | | | | | | | | | | J = 8.1 Hz, 1H), 4.85-4.71 (m, 1H), |
| | | | | | | | | | | | 4.14 (d, J = 17.4 Hz, 1H), 3.80 (s, |
| | | | | | | | | | | | 3H), 3.71 (d, J = 17.4 Hz, 1H), 2.48 |
| | | | | | | | | | | | (s, 3H), 2.07-1.98 (m, 1H), 1.89- |
| | | | | | | | | | | | 1.80 (m, 1H), 0.99 (t, J = 7.5 Hz, |
| | | | | | | | | | | | 3H). |
| 110 | 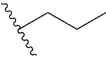 [SMILES: *CCC] | CH₃ | H | CH | Cl | Cl | CH₃ | H | H | CH | |
| 111 | 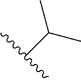 [SMILES: *C(C)C] | CH₃ | H | CH | Cl | Cl | CH₃ | H | H | CH | ¹H NMR (300 MHz, Chloroform-d) δ 7.95 (d, J = 2.1 Hz, 1H), 7.84 (d, J = 2.1 Hz, 1H), 7.55-7.44 (m, 3H), 6.25 (d, J = 8.7 Hz, 1H), 4.79-4.75 (m, 1H), 4.14 (d, J = 17.1 Hz, 1H), |
| | | | | | | | | | | | 3.79 (s, 3H), 3.71 (d, J = 17.1 Hz, |
| | | | | | | | | | | | 1H), 2.48 (s, 3H), 2.30-2.24 (m, |
| | | | | | | | | | | | 1H), 1.04 (d, J = 6.9 Hz, 3H), 0.96 |
| | | | | | | | | | | | (d, J = 6.9 Hz, 3H). |
| 112 | 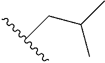 [SMILES: *CC(C)C] | CH₃ | H | CH | Cl | Cl | CH₃ | H | H | CH | |
| 113 | 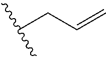 [SMILES: *CC=C] | CH₃ | H | CH | Cl | Cl | CH₃ | H | H | CH | |
| 114 | F | CH₃ | H | CH | Cl | Cl | CH₃ | H | H | CH | |
| 115 | 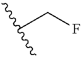 [SMILES: *CF] | CH₃ | H | CH | Cl | Cl | CH₃ | H | H | CH | |
| 116 | 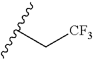 [SMILES: *CC(F)(F)F] | CH₃ | H | CH | Cl | Cl | CH₃ | H | H | CH | |
| 117 | 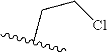 [SMILES: *CCCl] | CH₃ | H | CH | Cl | Cl | CH₃ | H | H | CH | |
| 118 | 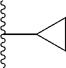 [SMILES: *C1CC1] | CH₃ | H | CH | Cl | Cl | CH₃ | H | H | CH | ¹H NMR (300 MHz, Chloroform-d) δ 7.95 (d, J = 2.1 Hz, 1H), 7.84 (d, J = 2.1 Hz, 1H), 7.57-7.43 (m, 3H), 6.34 (d, J = 7.5 Hz, 1H), 4.25- 4.20(m, 1H), 4.14 (d, J = 17.1 Hz, |
| | | | | | | | | | | | 1H), 3.81 (s, 3H), 3.71 (d, J = 17.1 |
| | | | | | | | | | | | Hz, 1H), 2.48 (s, 3H), 1.31-1.07 (m, |
| | | | | | | | | | | | 1H), 0.68-0.49 (m, 4H). |
| 119 | 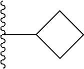 [SMILES: *C1CCC1] | CH₃ | H | CH | Cl | Cl | CH₃ | H | H | CH | |
| 120 | 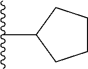 [SMILES: *C1CCCC1] | CH₃ | H | CH | Cl | Cl | CH₃ | H | H | CH | |
| 121 | 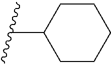 [SMILES: *C1CCCCC1] | CH₃ | H | CH | Cl | Cl | CH₃ | H | H | CH | |
| 122 | 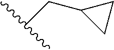 [SMILES: *CC1CC1] | CH₃ | H | CH | Cl | Cl | CH₃ | H | H | CH | |
| 123 | OH | CH₃ | H | CH | Cl | Cl | CH₃ | H | H | CH | |
| 124 | OCH₃ | CH₃ | H | CH | Cl | Cl | CH₃ | H | H | CH | ¹H NMR (300 MHz, Chloroform-d) δ |
| | | | | | | | | | | | 7.95 (d, J = 2.1 Hz, 1H), 7.84 (d, J = |
| | | | | | | | | | | | 2.1 Hz, 1H), 7.59-7.52 (m, 3H), |
| | | | | | | | | | | | 6.78 (d, J = 9.0 Hz, 1H), 5.72 (d, J = |
| | | | | | | | | | | | 9.0 Hz, 1H), 4.15 (d, J = 17.4 Hz, |
| | | | | | | | | | | | 1H), 3.87 (s, 3H), 3.71 (d, J = 17.4 |
| | | | | | | | | | | | Hz, 1H), 3.58 (s, 3H), 2.51 (s, 3H). |
| 125 | OCH₂CH₃ | CH₃ | H | CH | Cl | Cl | CH₃ | H | H | CH | |
| 126 | 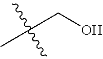 [SMILES: *CO] | CH₃ | H | CH | Cl | Cl | CH₃ | H | H | CH | |
| 127 | 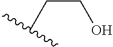 [SMILES: *CCO] | CH₃ | H | CH | Cl | Cl | CH₃ | H | H | CH | |
| 128 | 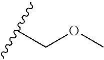 [SMILES: *COC] | CH₃ | H | CH | Cl | Cl | CH₃ | H | H | CH | |
| 129 | 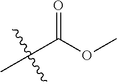 [SMILES: *C(=O)OC] | CH₃ | H | CH | Cl | Cl | CH₃ | H | H | CH | |
| 130 | 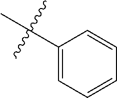 [SMILES: *c1ccccc1] | CH₃ | H | CH | Cl | Cl | CH₃ | H | H | CH | |
| 131 | 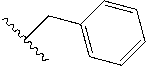 [SMILES: *Cc1ccccc1] | CH₃ | H | CH | Cl | Cl | CH₃ | H | H | CH | ¹H NMR (300 MHz, Chloroform-d) δ 7.94 (d, J = 2.1 Hz, 1H), 7.83 (d, J = 2.1 Hz, 1H), 7.52-7.43 (m, 2H), 7.32-7.25 (m, 4H), 7.16-7.10 (m, |
| | | | | | | | | | | | 2H), 6.21 (d, J = 7.8 Hz, 1H), 5.14- |
| | | | | | | | | | | | 4.98 (m, 1H), 4.12 (d, J = 17.4 Hz, |
| | | | | | | | | | | | 1H), 3.80 (s, 3H), 3.69 (d, J = 17.4 |
| | | | | | | | | | | | Hz, 1H), 3.38-3.27 (m, 1H), 3.22- |
| | | | | | | | | | | | 3.12 (m, 1H), 2.37 (s, 3H). |
| 132 | 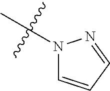 [SMILES: *n1cccn1] | CH₃ | H | CH | Cl | Cl | CH₃ | H | H | CH | |
| 133 | CH₃ | CH₃ | H | CH | Br | Cl | CH₃ | H | H | CH | |
| 134 | CH₃ | CH₃ | H | CH | I | Cl | CH₃ | H | H | CH | |
| 135 | CH₃ | CH₃ | H | CH | Cl | F | CH₃ | H | H | CH | |
| 136 | CH₃ | CH₃ | H | CH | Cl | Br | CH₃ | H | H | CH | |
| 137 | CH₃ | CH₃ | H | CH | Cl | I | CH₃ | H | H | CH | |
| 138 | CH₃ | CH₃ | H | CH | Cl | CF₃ | CH₃ | H | H | CH | |
| 139 | CH₃ | CH₃ | H | CH | Cl | Cl | H | H | H | CH | |
| 140 | CH₃ | CH₃ | H | CH | Cl | Cl | CH₂CH₃ | H | H | CH | ¹H NMR (300 MHz, Chloroform-d) δ |
| | | | | | | | | | | | 7.95 (d, J = 2.1 Hz, 1H), 7.84 (d, J = |
| | | | | | | | | | | | 2.1 Hz, 1H), 7.52-7.47 (m, 2H), |
| | | | | | | | | | | | 7.43 (d, J = 8.4 Hz, 1H), 6.37 (d, J = |
| | | | | | | | | | | | 7.5 Hz, 1H), 4.82-4.73 (m, 1H), |
| | | | | | | | | | | | 4.15 (d, J = 17.4 Hz, 1H), 3.79 (s, |
| | | | | | | | | | | | 3H), 3.72 (d, J = 17.4 Hz, 1H), 2.82 |
| | | | | | | | | | | | (q, J = 7.5 Hz, 2H), 1.52 (d, J = 7.5 |
| | | | | | | | | | | | Hz, 3H), 1.25 (t, J = 7.5 Hz, 3H). |
| 141 | CH₃ | CH₃ | H | CH | Cl | Cl | 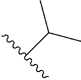 [SMILES: *C(C)C] | H | H | CH | ¹H NMR (300 MHz, Chloroform-d) δ 7.96 (d, J = 2.1 Hz, 1H), 7.85 (d, J = 2.1 Hz, 1H), 7.70-7.65 (m, 1H), 7.48-7.43 (m, 1H), 7.39 (d, J = 7.8 Hz, 1H), 6.34 (d, J = 7.5 Hz, 1H), |
| | | | | | | | | | | | 4.85-4.70 (m, 1H), 4.15 (d, J = 17.4 |
| | | | | | | | | | | | Hz, 1H), 3.79 (s, 3H), 3.71 (d, J = |
| | | | | | | | | | | | 17.4 Hz, 1H), 3.42-3.30 (m, |
| | | | | | | | | | | | 1H), 1.52 (d, J = 7.5 Hz, 3H), 1.32- |
| | | | | | | | | | | | 1.23 (m, 6H). |
| 142 | CH₃ | CH₃ | H | CH | Cl | Cl | 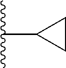 [SMILES: *C1CC1] | H | H | CH | |
| 143 | CH₃ | CH₃ | H | CH | Cl | Cl | F | H | H | CH | ¹H NMR (300 MHz, Chloroform-d) δ |
| | | | | | | | | | | | 8.19-8.14 (m, 1H), 7.91 (d, J = 6.3 |
| | | | | | | | | | | | Hz, 1H), 7.83 (d, J = 5.4 Hz, 1H), |
| | | | | | | | | | | | 7.59-7.44 (m, 2H), 7.37-7.32 (m, |
| | | | | | | | | | | | 1H), 4.88-4.73 (m, 1H), 4.13 (d, J = |
| | | | | | | | | | | | 17.4 Hz, 1H), 3.71 (d, J = 17.4 Hz, |
| | | | | | | | | | | | 1H), 3.82 (s, 3H), 1.56 (d, J = 1.2 |
| | | | | | | | | | | | Hz, 3H). |
| 144 | CH₃ | CH₃ | H | CH | Cl | Cl | Cl | H | H | CH | ¹H NMR (300 MHz, Chloroform-d) δ |
| | | | | | | | | | | | 7.93 (d, J = 2.1 Hz, 1H), 7.82 (d, J = |
| | | | | | | | | | | | 2.1 Hz, 1H), 7.80-7.68 (m, 1H), |
| | | | | | | | | | | | 7.65-7.58 (m, 1H), 7.62-7.59 (m, |
| | | | | | | | | | | | 1H), 6.90 (d, J = 7.2 Hz, 1H), 4.83- |
| | | | | | | | | | | | 4.73 (m, 1H) , 4.13 (d, J = 17.4 Hz, |
| | | | | | | | | | | | 1H), 3.80 (s, 3H), 3.71 (d, J = 17.4 |
| | | | | | | | | | | | Hz, 1H), 1.55 (d, J = 7.2 Hz, 3H). |
| 145 | CH₃ | CH₃ | H | CH | Cl | Cl | Br | H | H | CH | |
| 146 | CH₃ | CH₃ | H | CH | Cl | Cl | I | H | H | CH | |
| 147 | CH₃ | CH₃ | H | CH | Cl | Cl | CF₃ | H | H | CH | ¹H NMR (300 MHz, Chloroform-d) δ |
| | | | | | | | | | | | 8.01-7.81 (m, 4H), 7.63 (d, J = 7.8 |
| | | | | | | | | | | | Hz, 1H), 6.52 (d, J = 7.5 Hz, 1H), |
| | | | | | | | | | | | 4.83-4.72 (m, 1H), 4.17 (d, J = 17.4 |
| | | | | | | | | | | | Hz, 1H), 3.82-3.70 (m, 4H), 1.52 (d, |
| | | | | | | | | | | | J = 7.5 Hz, 3H). |
| 148 | CH₃ | CH₃ | H | CH | Cl | Cl | CH₂CF₃ | H | H | CH | |
| 149 | CH₃ | CH₃ | H | CH | Cl | Cl | CN | H | H | CH | |
| 150 | CH₃ | CH₃ | H | CH | H | Cl | F | H | H | CH | ¹H NMR (300 MHz, Chloroform-d) δ |
| | | | | | | | | | | | 8.19-8.14 (m, 1H), 7.81 (d, J = 2.1 |
| | | | | | | | | | | | Hz, 1H), 7.75 (d, J = 3.0 Hz, 1H), |
| | | | | | | | | | | | 7.70 (d, J = 4.2 Hz, 1H), 7.57-7.48 |
| | | | | | | | | | | | (m, 2H), 7.37-7.30 (m, 1H), 4.86- |
| | | | | | | | | | | | 4.76 (m, 1H), 4.14 (d, J = 17.4 Hz, |
| | | | | | | | | | | | 1H), 3.82 (s, 3H), 3.73 (d, J = 17.4 |
| | | | | | | | | | | | Hz, 1H), 1.56 (d, J = 1.2 Hz, 3H). |
| 151 | CH₃ | H | H | CH | Cl | Cl | CH₃ | H | H | CH | ¹H NMR (300 MHz, Chloroform-d) δ |
| | | | | | | | | | | | 11.35 (s, 1H), 7.94 (d, J = 2.1 Hz, |
| | | | | | | | | | | | 1H), 7.84 (d, J = 2.1 Hz, 1H), 7.52- |
| | | | | | | | | | | | 7.47 (m, 2H), 7.43 (d, J = 8.4 Hz, |
| | | | | | | | | | | | 1H), 6.39 (d, J = 7.2 Hz, 1H), 4.82- |
| | | | | | | | | | | | 4.72 (m, 1H), 4.13 (d, J = 17.1 Hz, |
| | | | | | | | | | | | 1H), 3.69 (d, J = 17.1 Hz, 1H), 2.48 |
| | | | | | | | | | | | (s, 3H), 1.57 (d, J = 7.2 Hz, 3H). |
| 152 | CH₂CH₃ | CH₃ | H | CH | F | Cl | CH₃ | H | H | CH | ¹H NMR (300 MHz, CDCl₃) δ 7.91 |
| | | | | | | | | | | | (dd, J = 6.0, 2.1 Hz, 1H), 7.75 (dd, |
| | | | | | | | | | | | J = 6.0, 2.1 Hz, 1H), 7.56-7.44 (m, |
| | | | | | | | | | | | 3H), 6.30 (d, J = 8.1 Hz, 1H), 4.82- |
| | | | | | | | | | | | 4.70 (m, 1H), 4.14 (d, J = 17.4 Hz, |
| | | | | | | | | | | | 1H), 3.80 (s, 3H), 3.71 (d, J = 17.4 |
| | | | | | | | | | | | Hz, 1H), 2.49 (s, 3H), 2.10-1.96 (m, |
| | | | | | | | | | | | 1H), 1.83 (m, 1H), 0.99 (t, J = 7.5 |
| | | | | | | | | | | | Hz, 3H). |
| 153 | CH₃ | CH₃ | H | CH | H | F | CH₃ | H | H | CH | ¹H NMR (300 MHz, Chloroform-d) δ |
| | | | | | | | | | | | 7.69-7.64(m, 1H), 7.61-7.55 (m, |
| | | | | | | | | | | | 1H), 7.54-7.50 (m, 2H), 7.49-7.38 |
| | | | | | | | | | | | (m, 2H), 6.34 (d, J = 7.5 Hz, 1H), |
| | | | | | | | | | | | 4.83-4.73 (m, 1H), 4.13 (d, J = 17.4 |
| | | | | | | | | | | | Hz, 1H), 3.80 (s, 3H), 3.73 (d, J = |
| | | | | | | | | | | | 17.4 Hz, 1H), 2.48 (s, 3H), 1.56 (d, |
| | | | | | | | | | | | J = 7.2 Hz, 3H). |
| 154 | CH₃ | CH₃ | H | N | Cl | Cl | CH₃ | H | H | CH | ¹H NMR (300 MHz, Chloroform-d) δ |
| | | | | | | | | | | | 8.69 (s, 1H), 8.50 (d, J = 7.8 Hz, |
| | | | | | | | | | | | 1H), 7.95 (d, J = 2.1 Hz, 1H), 7.87- |
| | | | | | | | | | | | 7.84 (m, 2H), 4.78-4.68 (m, 1H), |
| | | | | | | | | | | | 4.16 (d, J = 17.4 Hz, 1H), 3.79 (s, |
| | | | | | | | | | | | 3H), 3.73 (d, J = 17.4 Hz, 1H), 2.76 |
| | | | | | | | | | | | (s, 3H), 1.54 (d, J = 7.2 Hz, 3H). |
| 155 | CH₃ | CH₃ | H | N | H | Cl | CH₃ | H | H | CH | |
| 156 | CH₃ | CH₃ | H | N | F | Cl | CH₃ | H | H | CH | |
| 157 | CH₃ | CH₃ | CH₃ | N | Cl | Cl | CH₃ | H | H | CH | |
| 158 | CH₃ | CH₂CH₃ | H | N | Cl | Cl | CH₃ | H | H | CH | |
| 159 | CH₃ | 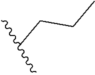 [SMILES: *CCC] | H | N | Cl | Cl | CH₃ | H | H | CH | |
| 160 | CH₃ | 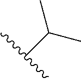 [SMILES: *C(C)C] | H | N | Cl | Cl | CH₃ | H | H | CH | |
| 161 | CH₃ | 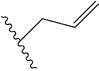 [SMILES: *CC=C] | H | N | Cl | Cl | CH₃ | H | H | CH | |
| 162 | CH | 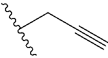 [SMILES: *CC#C] | H | N | Cl | Cl | CH₃ | H | H | CH | |
| 163 | CH₃ | 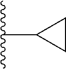 [SMILES: *C1CC1] | H | N | Cl | Cl | CH₃ | H | H | CH | |
| 164 | CH₃ | 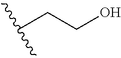 [SMILES: *CCO] | H | N | Cl | Cl | CH₃ | H | H | CH | |
| 165 | CH₃ | 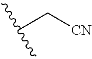 [SMILES: *CC#N] | H | N | Cl | Cl | CH₃ | H | H | CH | |
| 166 | CH₃ | 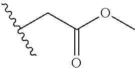 [SMILES: *CC(=O)OC] | H | N | Cl | Cl | CH₃ | H | H | CH | |
| 167 | CH₃ | 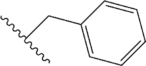 [SMILES: *Cc1ccccc1] | H | N | Cl | Cl | CH₃ | H | H | CH | |
| 168 | CH₃ | 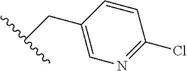 [SMILES: *Cc1ccc(Cl)nc1] | H | N | Cl | Cl | CH₃ | H | H | CH | |
| 169 | CH₃ | 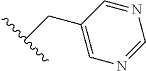 [SMILES: *Cc1cncnc1] | H | N | Cl | Cl | CH₃ | H | H | CH | |
| 170 | CH₂CH₃ | CH₃ | H | N | Cl | Cl | CH₃ | H | H | CH | |
| 171 | 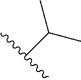 [SMILES: *C(C)C] | CH₃ | H | N | Cl | Cl | CH₃ | H | H | CH | |
| 172 | 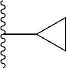 [SMILES: *C1CC1] | CH₃ | H | N | Cl | Cl | CH₃ | H | H | CH | |
| 173 | CH₃ | CH₃ | H | N | Cl | Cl | F | H | H | CH | |
| 174 | CH₃ | CH₃ | H | N | H | Cl | F | H | H | CH | |
| 175 | CH₃ | H | H | N | Cl | Cl | CH₃ | H | H | CH | |
| 176 | CH₂CH₃ | CH₃ | H | N | F | Cl | CH₃ | H | H | CH | |
| 177 | CH₃ | CH₃ | H | N | H | F | CH₃ | H | H | CH | |
| 178 | CF₃ | CH₃ | H | CH | F | Cl | CH₃ | H | H | CH | ¹H NMR (300 MHz, Chloroform-d) δ |
| | | | | | | | | | | | 8.76-8.73 (m, 1H), 8.50-8.42 (m, |
| | | | | | | | | | | | 2H), 7.93 (d, J = 6.3 Hz, 1H), 7.77 |
| | | | | | | | | | | | (d, J = 6.3 Hz, 1H), 7.74-7.70 (m, |
| | | | | | | | | | | | 1H), 7.52-7.46 (m, 1H), 4.20 (d, J = |
| | | | | | | | | | | | 17.4 Hz, 1H), 3.77 (d, J = 17.4 Hz, |
| | | | | | | | | | | | 1H), 2.72 (s, 3H), 1.60 (s, 3H). |
| 179 | 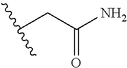 [SMILES: *CC(N)=O] | CH₃ | H | CH | Cl | Cl | CH₃ | H | H | CH | ¹H NMR (300 MHz, Chloroform-d) δ 7.94 (d, J = 2.1 Hz, 1H), 7.84 (d, J = 2.1 Hz, 1H), 7.57-7.43 (m, 3H), 7.18 (d, J = 8.1 Hz, 1H), 5.74 (s, 1H), 5.50 (s, 1H), 5.08-4.93 (m, |
| | | | | | | | | | | | 1H), 4.12 (d, J = 17.4 Hz, 1H), 3.80 |
| | | | | | | | | | | | (s, 3H), 3.69 (d, J = 17.4 Hz, 1H), |
| | | | | | | | | | | | 3.14-3.04 (m, 1H), 2.93-2.83 (m, |
| | | | | | | | | | | | 1H), 2.48 (s, 3H). |
| 180 | 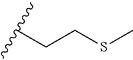 [SMILES: *CCSC] | CH₃ | H | CH | Cl | Cl | CH₃ | H | H | CH | ¹H NMR (300 MHz, Chloroform-d) δ 7.95 (d, J = 2.1 Hz, 1H), 7.84 (d, J = 2.1 Hz, 1H), 7.56-7.45 (m, 3H), 6.55 (d, J = 7.5 Hz, 1H), 4.91 (m, |
| | | | | | | | | | | | 1H), 4.14 (d, J = 17.1 Hz, 1H), 3.81 |
| | | | | | | | | | | | (s, 3H), 3.71 (d, J = 17.1 Hz, 1H), |
| | | | | | | | | | | | 2.60 (t, J = 7.2 Hz, 2H), 2.49 (s, |
| | | | | | | | | | | | 3H), 2.36-2.14 (m, 2H), 2.12 (s, |
| | | | | | | | | | | | 3H). |
| 181 | CH₃ | CH₃ | H | CH | F | F | CH₃ | H | H | CH | ¹H NMR (300 MHz, Chloroform-d) δ |
| | | | | | | | | | | | 7.76-7.68 (m, 1H), 7.64-7.61 (m, |
| | | | | | | | | | | | 1H), 7.56-7.49 (m, 2H), 7.47 (d, J = |
| | | | | | | | | | | | 8.4 Hz, 1H), 6.35 (d, J = 7.5 Hz, |
| | | | | | | | | | | | 1H), 4.83-4.73 (m, 1H), 4.13 (d, J = |
| | | | | | | | | | | | 17.1 Hz, 1H), 3.80 (s, 3H), 3.70 (d, |
| | | | | | | | | | | | J = 17.1 Hz, 1H), 2.48 (s, 3H), 1.53 |
| | | | | | | | | | | | (d, J = 7.2 Hz, 3H). |
| 182 | CH₃ | CH₃ | H | CH | F | Cl | CH₃ | H | H | N | ¹H NMR (300 MHz, Chloroform-d) δ |
| | | | | | | | | | | | 8.59 (s, 1H), 7.92-7.87 (m, 2H), |
| | | | | | | | | | | | 7.75 (d, J = 2.1 Hz, 1H), 6.43 (d, J = |
| | | | | | | | | | | | 7.2 Hz, 1H), 4.84-4.74 (m, 1H), |
| | | | | | | | | | | | 4.28 (d, J = 18.3 Hz, 1H), 3.88 (d, |
| | | | | | | | | | | | J = 18.3 Hz, 1H), 3.81 (s, 3H), 2.51 |
| | | | | | | | | | | | (s, 3H), 1.55 (d, J = 7.2 Hz, 3H). |
| 183 | CH₃ | CH₃ | H | CH | OCH₃ | Cl | CH₃ | H | H | CH | ¹H NMR (300 MHz, Chloroform-d) δ |
| | | | | | | | | | | | 7.89-7.84 (d, J = 1.8 Hz, 1H), 7.72 |
| | | | | | | | | | | | (d, J = 1.8 Hz, 1H), 7.55-7.49 (m, |
| | | | | | | | | | | | 2H), 7.46 (d, J = 8.4 Hz, 1H), 6.36 |
| | | | | | | | | | | | (d, J = 7.5 Hz, 1H), 4.84-4.72 (m, |
| | | | | | | | | | | | 1H), 4.11 (d, J = 17.4 Hz, 1H), 3.98 |
| | | | | | | | | | | | (s, 3H), 3.80 (s, 3H), 3.68 (d, J = |
| | | | | | | | | | | | 17.4 Hz, 1H), 2.47 (s, 3H), 1.53 (d, |
| | | | | | | | | | | | J = 7.2 Hz, 3H). |
| 184 | CH₃ | CH₃ | H | CH | Cl | Cl | 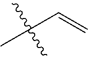 [SMILES: *C=C] | H | H | CH | ¹H NMR (300 MHz, DMSO-d₆) 8.92 (d, J = 6.9 Hz, 1H), 8.18 (s, 1H), 7.95-7.92 (m, 2H), 7.75-7.66 (m, 1H), 7.45 (d, J = 8.1 Hz, 1H), 6.96 |
| | | | | | | | | | | | (m, 1H), 5.96 (d, J = 17.4 Hz, 1H), |
| | | | | | | | | | | | 5.40 (d, J = 17.4 Hz, 1H), 4.51-4.43 |
| | | | | | | | | | | | (m, 3H), 3.66 (s, 3H), 1.35 (d, J = |
| | | | | | | | | | | | 6.9 Hz, 3H). |
| 185 | CH₃ | CH₃ | H | CH | Cl | Cl | H | Cl | H | CH | ¹H NMR (300 MHz, Chloroform-d) δ |
| | | | | | | | | | | | 7.92-7.90 (m, 2H), 7.85-7.69 (m, |
| | | | | | | | | | | | 3H), 6.76 (d, J = 7.2 Hz, 1H), 4.83- |
| | | | | | | | | | | | 4.73 (m, 1H), 4.34 (d, J = 17.1 Hz, |
| | | | | | | | | | | | 1H), 3.88 (d, J = 17.1 Hz, 1H), 3.81 |
| | | | | | | | | | | | (s, 3H), 1.54 (d, J = 6.9 Hz, 3H). |
| 186 | CH₃ | CH₃ | H | CH | Cl | Cl | H | Br | H | CH | ¹H NMR (300 MHz, Chloroform-d) δ |
| | | | | | | | | | | | 8.08 (d, J = 2.1 Hz, 1H), 7.93 (d, J = |
| | | | | | | | | | | | 2.1 Hz, 1H), 7.82-7.74 (m, 2H), |
| | | | | | | | | | | | 7.62 (d, J = 8.1 Hz, 1H), 6.79 (d, J = |
| | | | | | | | | | | | 7.2 Hz, 1H), 4.78 (m, 1H), 4.34 (d, |
| | | | | | | | | | | | J = 17.4 Hz, 1H), 3.88 (d, J = 17.4 |
| | | | | | | | | | | | Hz, 1H), 3.81 (s, 3H), 1.54 (d, J = |
| | | | | | | | | | | | 7.2 Hz, 3H). |
| 187 | CH₃ | CH₃ | H | CH | SCH₃ | Cl | CH₃ | H | H | CH | ¹H NMR (300 MHz, Chloroform-d) δ |
| | | | | | | | | | | | 7.95 (d, J = 2.1 Hz, 1H), 7.84 (d, J = |
| | | | | | | | | | | | 2.1 Hz, 1H), 7.52-7.44 (m, 3H), |
| | | | | | | | | | | | 6.39 (d, J = 7.2 Hz, 1H), 4.79-4.76 |
| | | | | | | | | | | | (m, 1H), 4.13 (d, J = 17.1 Hz, 1H), |
| | | | | | | | | | | | 3.79 (s, 3H), 3.72 (d, J = 17.1 Hz, |
| | | | | | | | | | | | 1H), 2.47 (s, 3H), 2.43 (s, 3H), 1.52 |
| | | | | | | | | | | | (d, J = 7.2 Hz, 3H). |
| 188 | H | CH₃ | H | CH | Cl | Cl | CH₃ | H | H | CH | ¹H NMR (300 MHz, Chloroform-d) δ |
| | | | | | | | | | | | 7.94 (d, J = 1.8 Hz, 1H), 7.84 (d, J = |
| | | | | | | | | | | | 1.8 Hz, 1H), 7.51-7.41 (m, 3H), |
| | | | | | | | | | | | 6.41 (t, J = 5.1 Hz, 1H), 4.20 (d, J = |
| | | | | | | | | | | | 5.1 Hz, 2H), 4.14 (d, J = 17.4 Hz, |
| | | | | | | | | | | | 1H), 3.79 (s, 3H), 3.73 (d, J = 17.4 |
| | | | | | | | | | | | Hz, 1H), 2.46 (s, 3H). |
| 189 | H | CH₂CH₃ | H | CH | F | Cl | CH₃ | H | H | CH | ¹H NMR (300 MHz, Chloroform-d) δ |
| | | | | | | | | | | | 7.93-7.89 (m, 1H), 7.77-7.73 (m, |
| | | | | | | | | | | | 1H), 7.52-7.47 (m, 2H), 7.43 (d, J = |
| | | | | | | | | | | | 8.4 Hz, 1H), 6.36 (t, J = 5.1 Hz, 1H), |
| | | | | | | | | | | | 4.29-4.19 (m, 4H), 4.13 (d, J = 17.1 |
| | | | | | | | | | | | Hz, 1H), 3.69 (d, J = 17.1 Hz, 1H), |
| | | | | | | | | | | | 2.48 (s, 3H), 1.31 (t, J = 6.9 Hz, |
| | | | | | | | | | | | 3H). |
| 190 | CH₃ | CH₃ | H | CH | Cl | Cl | CH₃ | H | H | N | |
| 191 | CH₃ | CH₃ | H | CH | F | Cl | CH₃ | H | CH₃ | CH | ¹H NMR (300 MHz, Chloroform-d) δ |
| | | | | | | | | | | | 7.93-7.89 (m, 1H), 7.77-7.73 (m, |
| | | | | | | | | | | | 1H), 7.52-7.50 (m, 2H), 7.44 (d, J = |
| | | | | | | | | | | | 8.4 Hz, 1H), 6.33 (s, 1H), 4.13 (d, |
| | | | | | | | | | | | J = 17.4 Hz, 1H), 3.80 (s, 3H), 3.70 |
| | | | | | | | | | | | (d, J = 17.4 Hz, 1H), 2.47 (s, 3H), |
| | | | | | | | | | | | 1.68 (s, 6H). |

Table A has the same structure as the above Table 1 does, except that the general formula I is replaced with the general formula I′ which has a chiral center

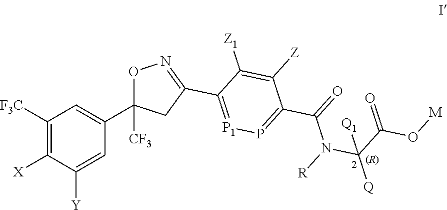

(i.e., Q and Q₁ are different); and in Table A, the items below the column heading “No.” are sequentially listed as 1 (2R)-190 (2R). For example, 1 (2R) corresponds to a compound that the 2-position is in R-configuration in Compound 1 in Table 1.

Table B has the same structure as the above Table 1 does, except that the general formula I is replaced by the general formula I″ with a chiral center

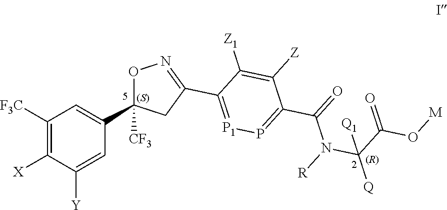

(i.e., Q and Q₁ are different); and in Table B, the items below the column heading “No.” are sequentially listed as 1 (5S, 2R)-190 (5S, 2R). For example, 1 (5S, 2R) corresponds to a compound that the 5-position is in S-configuration and the 2-position is in R-configuration in Compound 1 in Table 1.

Several methods for preparing the compounds of the present invention are illustrated in detail in the following schemes and examples. Raw materials can be purchased on the market or can be prepared by methods known in literatures or as shown in the detailed description. Those skilled in the art should understand that other synthetic routes can also be used to synthesize the compounds of the present invention. Although the specific raw materials and conditions in the synthetic route have been described below, they can be easily replaced with other similar raw materials and conditions. Various isomers and the like of the compounds resulting from these modifications or variants of the preparation method of the present invention are all included in the scope of the present invention. In addition, the preparation methods as described below can be further modified according to the disclosure of the present invention using conventional chemical methods well known to those skilled in the art. For example, protection for appropriate groups during the reaction, etc.

Examples of methods provided below are used to enhance further understanding of the preparation methods of the present invention, and the specific substances, varieties and conditions employed are determined to be further illustrations of the present invention and not to limit its reasonable scope. Reagents used in the synthesis of the compounds shown in the following tables are either commercially available or can be easily prepared by those of ordinary skill in the art.

Examples of representative compounds are as follows. The synthesis methods of other compounds are similar and will not be described in detail here.

### 1. Synthesis of Compound 3 (5S, 2R)

(1) A 50 ml round-bottom flask was taken and added with 10 ml of DCM, Compound 3-1 (0.3 g, 1 eq), D-alanine methyl ester hydrochloride (0.35 g, 1.5 eq), 1-ethyl-3-(3′-dimethylaminopropyl)carbodiimide hydrochloride (EDCI, 0.64 g, 2 eq) and 2-(7-azabenzotriazol-1-yl)-N,N,N′,N′-tetramethyluronium hexafluorophosphate (HATU, 1.28 g, 2 eq). The reaction solution was reacted at 45° C. for 16 h. After the reaction was completed, the solvent was removed by decompressing concentration, and the white solid product 3-2 (367 mg, 82% yield) was obtained by silica gel column chromatography.

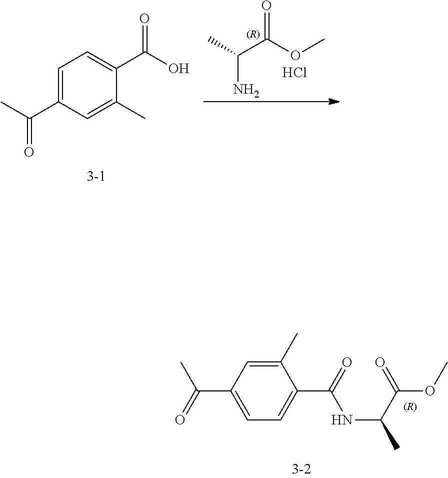

(2) A 50 ml round-bottom flask was taken and added with 10 ml of dichloroethane, Compound 3-2 (0.15 g, 1 eq), Compound 3-3 (0.18 g, 1 eq), potassium carbonate (0.09 g, 1.2 eq) and triethylamine (0.07 g, 1.2 eq). The reaction solution was heated up to 80° C. and reacted for 16 h. After the reaction was completed, the solvent was removed by decompressing concentration, and the white solid product 3-4 (156 mg, 49% yield) was obtained by silica gel column chromatography.

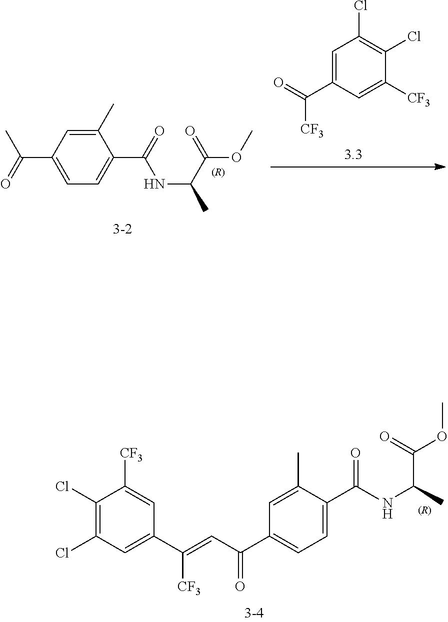

(3) A 50 ml round-bottom flask was taken and added with 10 ml of DCM and Compound 3-4 (0.15 g, 1 eq). The catalyst (35 mg) was added at 0° C. and stirred evenly; 0.06 g (6 eq) of sodium hydroxide was dissolved in 5 times the volume of water, added with hydroxyamine hydrochloride (0.09 g, 5 eq, containing 50% water), and stirred for 30 min. The obtained mixture was slowly dropped into the reaction system and reacted at 0° C. for 2 h. After the reaction was completed, the solution was extracted with DCM. The organic layer was concentrated by decompression to remove the solvent. The white solid product 3 (5S, 2R) (82 mg, 97% purity, ee 53, 53% yield) was obtained by silica gel column chromatography.

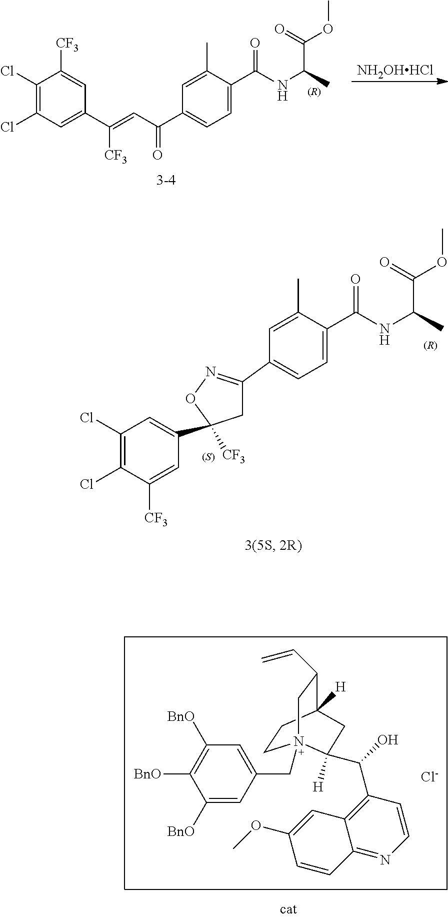

### 2. Synthesis of Compound 3 (2R)

Compound 3-5 (1 g, 1 eq) was dissolved in 25 ml of DCM, sequentially added with D-alanine methyl ester hydrochloride (574.17 mg, 2 eq), HOBT (416.89 mg, 1.5 eq) and triethylamine (312.20 mg, 1.5 eq), stirred for 5 min, then added with EDCI (591.44 mg, 1.5 eq) and reacted at room temperature for 3 h. After the reaction was completed, water was added to the system for extraction. The aqueous layer was further extracted twice with DCM. The organic phase was washed with saturated sodium chloride solution for 3-5 times, added with anhydrous sodium sulfate for drying and concentrated by decompression to remove the solvent. The white solid product 3 (2R) (800 mg, 95% purity, 68% yield) was obtained by normal phase purification.

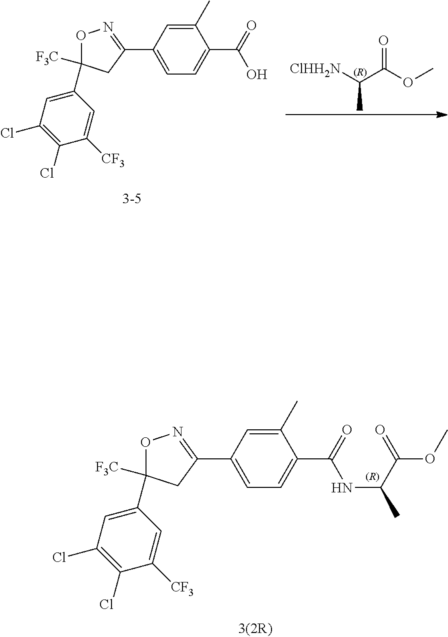

### 3. Synthesis of Compound 151 (2R)

Compound 3 (2R) (0.5 g, 0.87 mmol) was dissolved in 20 ml of THF, added dropwise with LiOH (83.83 mg, 4 eq, 3.5 mmol) aqueous (5 ml) solution under the ice bath and reacted at room temperature for 12 h. After the LCMS detected that the reaction had been completed, the THE was removed by decompressing concentration. Then 2 M diluted hydrochloric acid was added to adjust the pH to 4, and EA was added for extraction. The organic phase was concentrated by decompression. Compound 151 (2R) (400 mg, 82% yield, 95% purity) was obtained by normal phase purification.

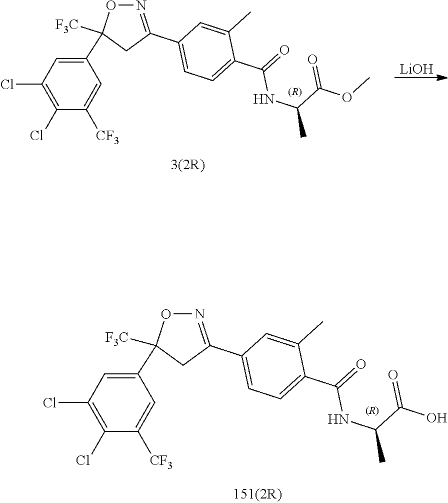

### 4. Synthesis of Compound 4 (2R)

Compound 151 (2R) (100 mg, 1 eq) was dissolved in 5 mL of DMF, added sequentially with cesium carbonate (175 mg, 3 eq) and methyl iodide (128 mg, 5 eq) under stirring, heated up to 45° C. and reacted for 12 h. After the LCMS detected that the reaction had been completed, the reaction solution was added with water and then extracted with ethyl acetate. The organic phase was washed with saturated saline solution, dried with anhydrous sodium sulfate and concentrated by decompression. Compound 4 (2R) (90 mg, 86% yield, 96% purity) was obtained by normal phase purification.

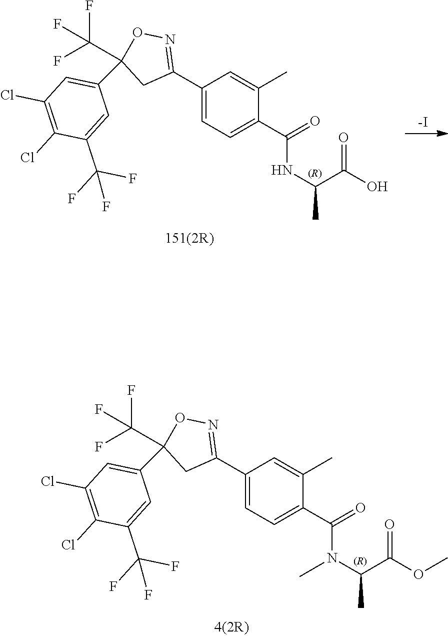

### 5. Synthesis of Compound 30 (2R)

Compound 151 (2R) (0.15 g, 269.17 umol) was dissolved in 10 mL of DMF, added with ethyl iodide (62.97 mg, 1.5 eq, 403.75 umol) and potassium carbonate (111.60 mg, 3 eq, 807.51 umol), and reacted at room temperature for 6 h. After the LCMS detected that the reaction had been completed, the reaction solution was extracted with water and EA. The aqueous phase was extracted twice with EA. The organic phases were combined, washed with saturated sodium chloride solution for 3 times and concentrated by decompression. Compound 30 (2R) (100 mg, 63% yield, 95% purity) was obtained by normal phase purification.

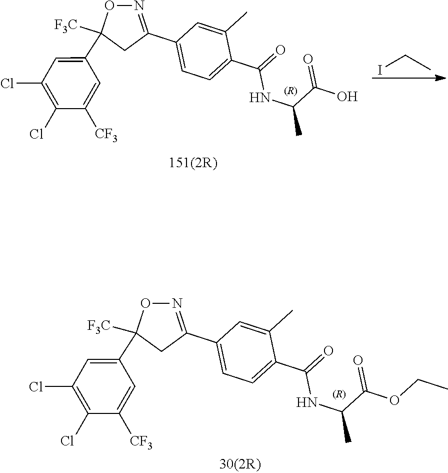

### 6. Synthesis of Compound 80 (2R)

A 50 ml round-bottom flask was taken and added with 15 ml of DCM solvent, acid compound 151 (2R) (0.18 g, 1 eq), acetone oxime (0.04 g, 1.5 eq), EDCI (0.09 g, 1.5 eq) and HOBT (0.07 g, 1.5 eq). The reaction solution was displaced by nitrogen, and stirred at room temperature for 1 h. After the reaction was completed, dichloromethane and water were used for extraction for three times. The organic phase was concentrated by decompression to remove the solvent. The white solid product 80 (2R) (120 mg, 61%) was obtained by normal phase separation.

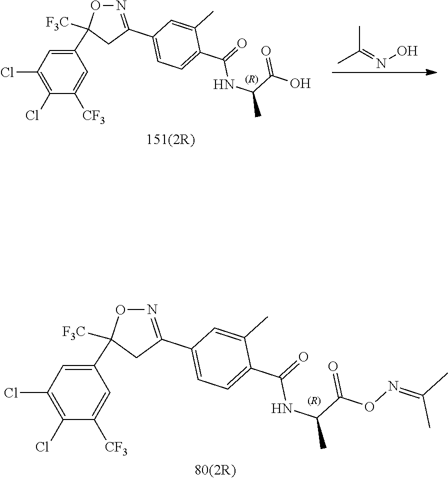

### 7. Synthesis of Compound 154 (2R)

Compound 154-1 (800 mg, 1.0 eq) was added to EtOH, and NH₂OH HCl (340 mg, 1.1 eq) was added dropwise at 0° C. After the addition, the reaction was carried out at room temperature for 1 h. After the In-Process Control was completed, the solvent was removed by decompressing concentration. The reaction solution was added with water and added with EA to extract twice. The organic phase was washed twice with saturated saline solution, dried with anhydrous sodium sulfate, and concentrated by decompression to obtain the crude product 154-2 (740 mg) for direct use in the next step.

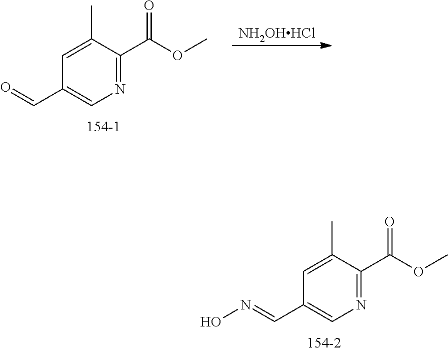

Compound 154-2 (740 mg, 1 eq) was dissolved in 10 ml of DMF, heated up to 35° C., added with NCS (560 mg, 1.1 eq) and reacted for 1.5 h. After the In-Process Control was completed, the reaction solution was cooled down to room temperature, added with water and EA, and extracted twice with EA. The organic phase was washed twice with saturated saline solution, dried with anhydrous sodium sulfate, and concentrated by decompression to obtain the crude product 154-3 (850 mg) for direct use in the next step.

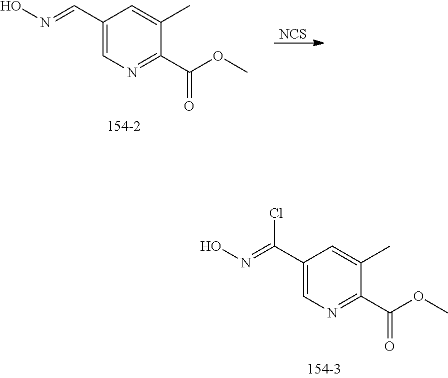

Compound 154-3 (850 mg, 1 eq) was dissolved in 10 ml of DCM, added with triethylamine (750 mg, 2 eq) and added dropwise with Compound a (1.15 g, 1 eq) under the ice bath. After the addition, the reaction was carried out for 2 h. After the In-Process Control was completed, water and DCM were added for extraction. The organic phase was purified by normal phase chromatography to obtain Compound 154-4 (480 mg).

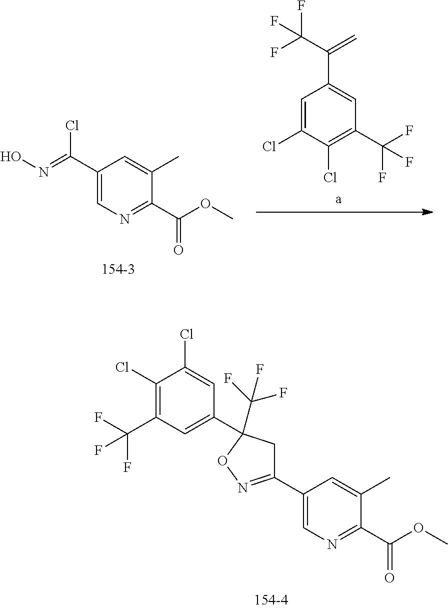 [SMILES: COC(=O)c1ncc(/C(Cl)=N/O)cc1C ; C=C(c1cc(Cl)c(Cl)c(C(F)(F)F)c1)C(F)(F)F ; COC(=O)c1ncc(C2=NOC(c3cc(Cl)c(Cl)c(C(F)(F)F)c3)(C(F)(F)F)C2)cc1C]

Compound 154-4 (480 mg, 1 eq) was dissolved in 20 ml of THF, added dropwise with NaOH (154 mg, 4 eq) aqueous solution under the ice bath, heated up to 60° C. and reacted for 4 h. After the In-Process Control was completed, the reaction solution was cooled down to room temperature. And 2 M diluted hydrochloric acid was added to adjust the pH to 4 and EA was added for extraction. The organic phase was concentrated by decompression to obtain Compound 154-5 (400 mg).

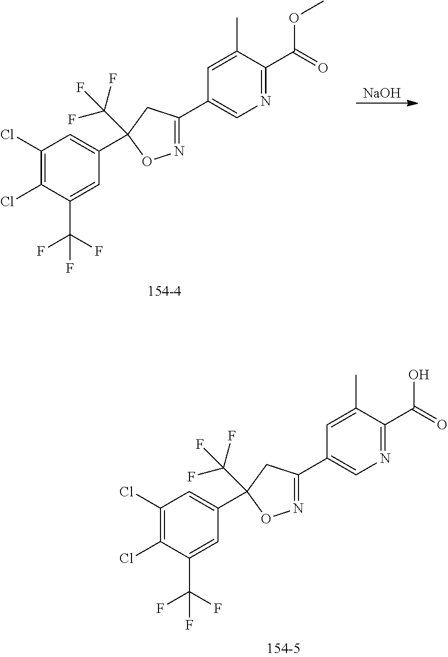

Compound 154-5 (100 mg, 1 eq) was dissolved in 10 mL of DCM, sequentially added with D-alanine methyl ester hydrochloride (42 mg, 1.5 eq), HOBT (41 mg, 1.5 eq) and triethylamine (41 mg, 2 eq), stirred at room temperature for 5 min, and finally added with EDCI (59 mg, 1.5 eq) and reacted at room temperature for 0.5 h. After the In-Process Control was completed, water and DCM were added for extraction. The aqueous phase was further extracted twice with DCM. The organic phases were combined, washed with saturated sodium chloride solution for 3 times and concentrated by decompression. Compound 154 (2R) (60 mg) was obtained by normal phase purification.

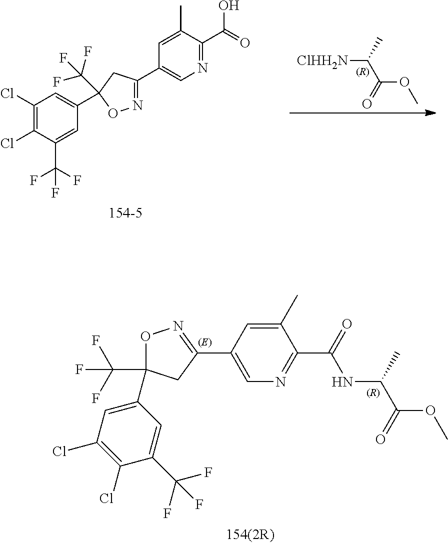

### 8. Synthesis of Compound 144 (2R)

The 50 ml of DMF was added with the 144-1 (1.15 g, 1.0 eq). The NCS (1.05 eq) was added dropwise to the reaction solution at 35° C. After 1 hour of reaction, the LCMS detected disappearance of the raw materials and the main peak was the product peak. The EA and water were added for extraction. The reaction solution was extracted with ethyl acetate and washed with saturated saline solution (100 ml*3). The organic phase was concentrated to obtain the crude product 144-2 (1.1 g) for direct use in the next step.

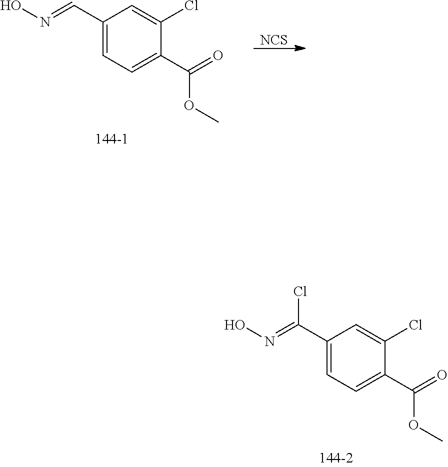

At 0° C., Compound a (1.2 eq) was dissolved in DCM, and added dropwise with triethylamine (2 eq). After the addition, Compound 144-2 (0.2 g, 1 eq) was then added dropwise, heated up to 40° C., and stirred overnight. The LCMS exhibited the presence of the product. The reaction solution was washed with saturated saline solution (150 ml*3), and added with EA for extraction to obtain an oily matter. Compound 144-3 (0.23 g, 54.0% yield) was obtained by column chromatography separation.

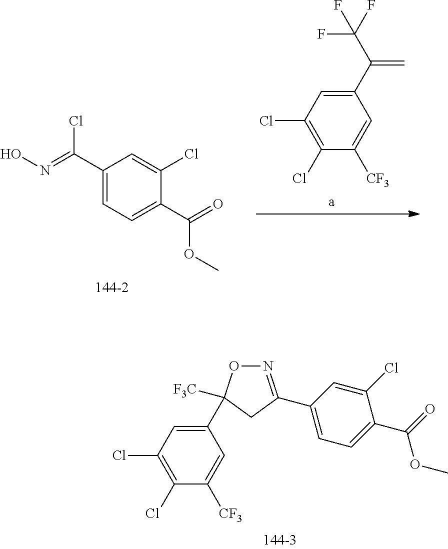 [SMILES: COC(=O)c1ccc(/C(Cl)=N/O)cc1Cl ; C=C(c1cc(Cl)c(Cl)c(C(F)(F)F)c1)C(F)(F)F ; COC(=O)c1ccc(C2=NOC(c3cc(Cl)c(Cl)c(C(F)(F)F)c3)(C(F)(F)F)C2)cc1Cl]

Compound 144-3 (0.23 g, 1 eq) was added to a mixed solvent of 12 ml of THF and 4 ml of water. Sodium hydroxide (3 eq) was added in batches at room temperature under stirring, heated up to 40° C. and stirred overnight. The LCMS detected the presence of the product. The solvent was concentrated, added with hydrochloric acid to adjust the pH to acidic, and added with ethyl acetate for extraction. The organic phase was concentrated to obtain Compound 144-4 (0.2 g, 89% yield).

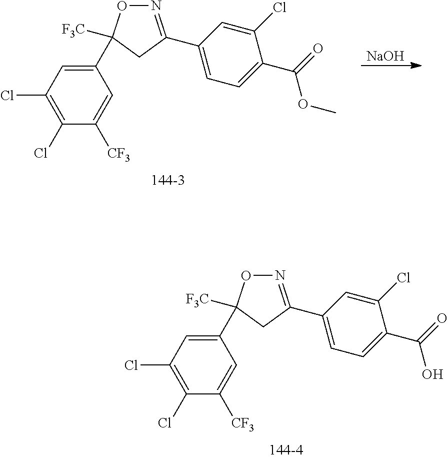

Compound 144-4 (100 mg, 1.0 eq), D-alanine methyl ester hydrochloride (1.1 eq) and 8 mL of dichloromethane were added into a 100 mL single-necked flask, and then triethylamine (2.0 eq) and HOBT (1.5 eq) were added and stirred under the ice bath for 5 min. EDCI (1.5 eq) was added and continued to stir for 1 h. The LCMS detection showed that the raw materials had been almost completely consumed. A major new peak was formed, and the MS signal was correct. The reaction solution was added with 20 ml of water, separated and extracted for three times. The organic phases were combined, washed with saturated saline solution for three times, dried by anhydrous sodium sulfate and then column-purified to obtain Compound 144 (2R) (90 mg, 76.7% yield) (white solid).

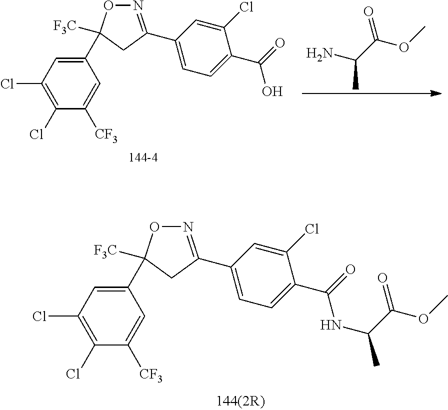

### Evaluation of Biological Activity

### (1) Insecticidal Activity Assay:

The compound of the present invention was dissolved in acetone, and then diluted with water into liquids with gradients of different concentrations (ppm). Corn leaves were soaked in liquids of different concentrations for about 20 seconds, air-dried and then placed in petri dishes where 3^{rd} instar Spodoptera frugiperda were placed, respectively; kale leaves were soaked in liquids of different concentrations for about 20 seconds, air-dried and then placed in petri dishes where 3^{rd} instar Spodoptera frugiperda were placed, respectively; peanut leaves were soaked in liquids of different concentrations for about 20 seconds, air-dried and then placed in petri dishes where Tetranychus urticae (adult mite) and Aphis craccivora (adult aphid) were placed, respectively; water bamboo leaves were soaked in liquids of different concentrations for about 20 seconds, air-dried and then placed in petri dishes where 4^{th} instar Chilo suppressalis were placed, respectively. Each petri dish was placed with 10 insects, covered and then stored at 25° C. in a constant temperature room. The number of dead insects were checked after 48 h. The dead insect rate was calculated by the formula: Death rate (%)=(Number of dead insects/Number of test insects)×100. Furthermore, the experiment was conducted in two replications.

TABLE 2
Results of insecticidal tests

| | Spodoptera | | Spodoptera | | Aphis | | Tetranychus | | Chilo | |
|---|---|---|---|---|---|---|---|---|---|---|
| | frugiperda | | exigua | | craccivora | | urticae | | suppressalis | |
| | | Liquid | | Liquid | | Liquid | | Liquid | | Liquid |
| | Death | concen- | Death | concen- | Death | concen- | Death | concen- | Death | concen- |
| | rate/ | tration/ | rate/ | tration/ | rate/ | tration/ | rate/ | tration/ | rate/ | tration/ |
| No. | % | ppm | % | ppm | % | ppm | % | ppm | % | ppm |
| 1 (2R) | 100 | 0.3 | 90 | 0.3 | 83 | 8.1 | 83 | 8.1 | 100 | 0.4 |
| 2 (2R) | 100 | 0.3 | 90 | 0.3 | 90 | 8.1 | 100 | 0.9 | 100 | 0.4 |
| 2 | 100 | 0.3 | 100 | 0.3 | 100 | 8.1 | 100 | 0.9 | 100 | 0.4 |
| (5S, 2R) | | | | | | | | | | |
| 3 (2R) | 100 | 0.3 | 77 | 0.3 | 93 | 8.1 | 100 | 0.9 | 100 | 0.4 |
| 4 (2R) | 100 | 0.3 | N | N | N | N | N | N | N | N |
| 30 (2R) | 90 | 0.3 | N | N | N | N | 80 | 0.9 | N | N |
| 41 (2R) | N | N | N | N | N | N | 75 | 0.9 | N | N |
| 43 (2R) | N | N | N | N | N | N | 85 | 0.9 | N | N |
| 57 (2R) | N | N | N | N | N | N | 90 | 8.1 | N | N |
| 80 (2R) | 100 | 0.3 | 100 | 0.3 | 100 | 8.1 | 100 | 8.1 | 100 | 0.4 |
| 111 | N | 0.3 | 60 | 0.3 | 40 | 8.1 | N | 8.1 | N | 0.4 |
| 118 | N | 0.3 | 60 | 0.3 | 40 | 8.1 | N | 8.1 | N | 0.4 |
| 124 | 80 | 0.3 | 70 | 0.3 | N | N | 77 | 8.1 | N | N |
| 131 (2R) | N | N | N | N | N | N | 80 | 8.1 | N | N |
| 140 (2R) | 100 | 0.3 | 100 | 0.3 | 100 | 8.1 | 100 | 8.1 | 100 | 0.4 |
| 141 (2R) | 90 | 0.3 | N | N | N | N | N | N | N | N |
| 144 (2R) | 100 | 0.3 | 100 | 0.3 | 100 | 8.1 | 100 | 8.1 | 100 | 0.4 |
| 147 (2R) | 100 | 0.3 | 100 | 0.3 | 100 | 8.1 | 100 | 8.1 | 100 | 0.4 |
| 151 (2R) | N | N | N | N | N | N | 100 | 8.1 | N | N |
| 151 | N | N | N | N | N | N | 100 | 8.1 | N | N |
| (5S, 2R) | | | | | | | | | | |
| 154 (2R) | 100 | 0.3 | 77 | 0.3 | 93 | 8.1 | 100 | 0.9 | 100 | 0.4 |
| 178 | N | N | N | N | N | N | 50 | 8.1 | N | N |
| 179 (2R) | N | N | N | N | N | N | 80 | 8.1 | N | N |
| 180 (2R) | N | N | N | N | N | N | 45 | 8.1 | N | N |
| 181 (2R) | N | N | N | N | N | N | 70 | 8.1 | N | N |
| 182 (2R) | N | N | N | N | N | N | 100 | 8.1 | N | N |
| 183 (2R) | 100 | 0.3 | 100 | 0.3 | N | N | N | N | N | N |
| 183 | N | N | N | N | N | N | 70 | 8.1 | N | N |
| (5S, 2R) | | | | | | | | | | |
| 184 | N | N | N | N | N | N | 100 | 8.1 | N | N |
| 185 (2R) | N | N | N | N | N | N | 50 | 8.1 | N | N |
| 187 (2R) | N | N | N | N | N | N | 63 | 8.1 | N | N |
| 188 (5S) | 100 | 0.3 | 100 | 0.3 | 100 | 8.1 | 100 | 8.1 | 100 | 0.4 |
| Control | 70 | 0.3 | 30 | 0.3 | 20 | 8.1 | 25 | 8.1 | 40 | 0.4 |
| com- | | | | | | | | | | |
| pound A | | | | | | | | | | |

Note:
N represents no data; Control compound A: 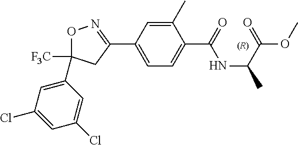
(derived from the patent WO2005/085216).

### (2) Assay of Insecticidal Activity of Compositions:

### 2.1) Experimental Conditions and Operation Procedures

Test targets: Spodoptera frugiperda (3^{rd} instar) and Aphis gossypii (adult aphid)

Referring to the above-mentioned Insecticidal activity assay, when the test target was Aphis gossypii, the zucchini leaves were selected.

### 2.2) Qualitative Evaluation of Synergism

Different ratios within the selected ratio range were set for toxicity determination, and the optimal ratio was selected according to synergistic effects. When the synergistic effect >0, it indicated a synergistic effect; when the synergistic effect is close to 0, it indicated an addition effect; when the synergistic effect <0, it indicated an antagonistic effect.

$$Synergistic effect = Actual death rate - Theoretical death rate \\ Theoretical death rate = 1 - ( 1 - {P}_{1} ) ( 1 - {P}_{2} )$$

wherein, P₁, P₂—Death rate by each individual agent in a mixed formulation.

TABLE 3
Qualitative evaluation results of synergisms of compositions

| | | | | | Death rate by | | | |
|---|---|---|---|---|---|---|---|---|
| | A + B | | Death rate by | Death rate by | mixed | Theoretical | Synergistic | |
| | Dosage/ | | individual A | individual B | formulation | death rate | effect | |
| A + B | ppm | A:B | (%) | (%) | (%) | (%) | (%) | Test target |
| 3 (2R) + | 0.1 + 0.5 | 1:5 | 72 | 50 | 98 | 86 | 12 | Spodoptera |
| Chlorantraniliprole | | | | | | | | frugiperda |
| 3 (2R) + Spinetoram | 0.1 + 0.5 | 1:5 | 72 | 62 | 99 | 89 | 10 | Spodoptera |
| | | | | | | | | frugiperda |
| 3 (2R) + lambda- | 0.1 + 2 | 1:20 | 72 | 41 | 94 | 83 | 11 | Spodoptera |
| cyhalothrin | | | | | | | | frugiperda |
| 3 (2R) + Emamectin- | 0.1 + 2 | 1:20 | 72 | 37 | 98 | 82 | 16 | Spodoptera |
| benzoate | | | | | | | | frugiperda |
| 3 (2R) + Abamectin | 0.1 + 100 | 1:1000 | 72 | 25 | 89 | 79 | 10 | Spodoptera |
| | | | | | | | | frugiperda |
| 3 (2R) + Indoxacarb | 0.1 + 0.5 | 1:5 | 72 | 36 | 96 | 82 | 14 | Spodoptera |
| | | | | | | | | frugiperda |
| 3 (2R) + Lufenuron | 0.1 + 50 | 1:500 | 72 | 38 | 94 | 83 | 11 | Spodoptera |
| | | | | | | | | frugiperda |
| 3 (2R) + Chlorfenapyr | 0.1 + 50 | 1:500 | 72 | 59 | 99 | 89 | 10 | Spodoptera |
| | | | | | | | | frugiperda |
| 3 (2R) + Spirotetramat | 0.1 + 100 | 1:1000 | 72 | 47 | 98 | 85 | 13 | Spodoptera |
| | | | | | | | | frugiperda |
| 3 (2R) + Triflumezopyrim | 0.1 + 100 | 1:1000 | 72 | 33 | 96 | 81 | 15 | Spodoptera |
| | | | | | | | | frugiperda |
| 3 (2R) + Diafenthiuron | 0.1 + 100 | 1:1000 | 72 | 49 | 97 | 86 | 11 | Spodoptera |
| | | | | | | | | frugiperda |
| 3 (2R) + Cyetpyrafen | 0.1 + 100 | 1:1000 | 72 | 30 | 95 | 80 | 15 | Spodoptera |
| | | | | | | | | frugiperda |
| 3 (2R) + Acephate | 0.1 + 300 | 1:3000 | 72 | 38 | 96 | 83 | 13 | Spodoptera |
| | | | | | | | | frugiperda |
| 3 (2R) + Bifenthrin | 0.1 + 1 | 1:10 | 72 | 22 | 89 | 78 | 11 | Spodoptera |
| | | | | | | | | frugiperda |
| 3 (2R) + Methoxyfenozide | 0.1 + 2 | 1:20 | 72 | 31 | 93 | 81 | 12 | Spodoptera |
| | | | | | | | | frugiperda |
| 3 (2R) + Spiromesifen | 0.1 + 100 | 1:1000 | 72 | 49 | 97 | 86 | 11 | Spodoptera |
| | | | | | | | | frugiperda |
| 3 (2R) + Fipronil | 0.1 + 1 | 1:10 | 72 | 43 | 96 | 84 | 12 | Spodoptera |
| | | | | | | | | frugiperda |
| 3 (2R) + Spiropidion | 0.1 + 2 | 1:20 | 72 | 39 | 96 | 83 | 13 | Spodoptera |
| | | | | | | | | frugiperda |
| 3 (2R) + Buprofezin | 0.1 + 10 | 1:100 | 72 | 36 | 98 | 82 | 16 | Spodoptera |
| | | | | | | | | frugiperda |
| 3 (2R) + Pymetrozine | 0.1 + 50 | 1:500 | 72 | 30 | 92 | 80 | 12 | Spodoptera |
| | | | | | | | | frugiperda |
| 3 (2R) + Clothianidin | 0.1 + 5 | 1:50 | 38 | 42 | 78 | 64 | 14 | Aphis |
| | | | | | | | | gossypii |
| 3 (2R) + Nitenpyram | 0.1 + 1 | 1:10 | 38 | 36 | 80 | 60 | 20 | Aphis |
| | | | | | | | | gossypii |
| 3 (2R) + Acetamiprid | 0.1 + 1 | 1:10 | 38 | 52 | 92 | 70 | 22 | Aphis |
| | | | | | | | | gossypii |
| 3 (2R) + Afidopyropen | 0.1 + 1 | 1:10 | 38 | 68 | 97 | 80 | 17 | Aphis |
| | | | | | | | | gossypii |
| 3 (2R) + Cyantraniliprole | 0.1 + 5 | 1:50 | 38 | 36 | 74 | 60 | 14 | Aphis |
| | | | | | | | | gossypii |
| 3 (2R) + Dinotefuran | 0.1 + 5 | 1:50 | 38 | 40 | 81 | 63 | 18 | Aphis |
| | | | | | | | | gossypii |
| 3 (2R) + Ethiprole | 0.1 + 5 | 1:50 | 38 | 33 | 72 | 58 | 14 | Aphis |
| | | | | | | | | gossypii |
| 3 (2R) + Imidacloprid | 0.1 + 1 | 1:10 | 38 | 53 | 87 | 71 | 16 | Aphis |
| | | | | | | | | gossypii |
| 3 (2R) + Sulfoxaflor | 0.1 + 2 | 1:20 | 38 | 46 | 79 | 67 | 12 | Aphis |
| | | | | | | | | gossypi |
| 3 (2R) + Thiamethoxam | 0.1 + 1 | 1:10 | 38 | 64 | 88 | 78 | 10 | Aphis |
| | | | | | | | | gossypii |
| 3 (2R) + Tolfenpyrad | 0.1 + 1 | 1:10 | 38 | 35 | 75 | 60 | 15 | Aphis |
| | | | | | | | | gossypii |
| 3 (2R) + dimpropyridaz | 0.1 + 2 | 1:20 | 38 | 48 | 82 | 68 | 14 | Aphis |
| | | | | | | | | gossypii |

Meanwhile, it has been found after extensive experimentations that, many of the compounds of the present invention and compositions thereof have good controlling activity against agricultural pests such as Lepidoptera (e.g., Ostrinia nubilalis, Chilo suppressalis, Plutella xylostella, Spodoptera litura, Spodoptera exigua, Helicoverpa armigera, Spodoptera frugiperda, Mythimna separata, etc.), Homoptera (e.g., Aphis gossypii, Lipaphis erysimi, Acyrthosiphon pisum, Aphis craccivora, Apolygus lucorum, etc.), Acarina (e.g., Tetranychus urticae, Tetranychus truncatus, Tetranychus turkestani, etc.), Diptera (e.g., Bradysia odoriphaga, etc.), Coleoptera (e.g., Phyllotreta striolata, Phaedon brassicae, etc.) and Thripidae (e.g., Thrips palmi, Thrips alliorum, Thrips tabaci, etc.) and the like, as well as sanitary pests such as Blattidae (e.g., termite, cockroach, etc.), Muscidae (e.g., fly, mosquito, etc.) and the like, not only possessing the characteristics of broad spectrum, high efficiency and strong systemic property, but also capable of controlling resistant pest insects, which have certain commercial values.

1. An isoxazoline-substituted benzamide compound as shown in Formula I, a stereoisomer and an agriculturally or veterinarily acceptable salt thereof: 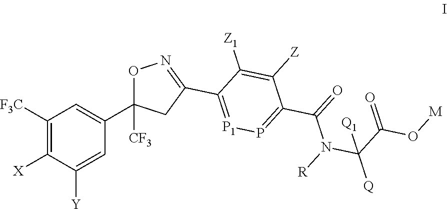
wherein, X represents hydrogen, halogen, alkoxy or alkylthio;
Y represents halogen or haloalkyl;
Z and Z₁ each independently represent hydrogen, halogen, alkyl, alkenyl, alkynyl, cycloalkyl, haloalkyl or cyano;
P and P₁ each independently represent CH or N;
Q and Q₁ each independently represent hydrogen, halogen, hydroxyl, cyano, amino, nitro, formyl, cyanoalkyl, hydroxyalkyl, alkyl, alkenyl, alkynyl, cycloalkyl, cycloalkylalkyl, alkoxy, alkylthio, alkylamino, haloalkoxy, haloalkylthio, alkylcarbonyl, alkoxycarbonyl, alkoxyalkyl, haloalkoxyalkyl, alkylaminoalkyl, aryl, heterocyclyl, arylalkyl or heterocyclylalkyl; wherein, the “alkyl”, “alkenyl” or “alkynyl” are each independently unsubstituted or substituted by at least one group of halogen, —OR₁, —SR₁, —(CO)OR₁, —(CO)N(R₁)₂, —(SO₂)R₁ or —N(R₁)₂; the “cycloalkyl”, “cycloalkylalkyl”, “aryl”, “heterocyclyl”, “arylalkyl” or “heterocyclylalkyl” are each independently unsubstituted or substituted by at least one group selected from oxo, halogen, cyano, nitro, alkyl, alkenyl, alkynyl, cycloalkyl, haloalkyl, haloalkenyl, haloalkynyl, halocycloalkyl, cycloalkyl substituted by alkyl, —OR₁, —SR₁, —(CO)OR₁, —(SO₂)R₁, —N(R₁)₂ or —O-alkyl-(CO)OR₁, or two adjacent carbon atoms on the ring form a fused ring with —OCH₂CH₂— or —OCH₂O— that is unsubstituted or substituted by halogen;
M represents hydrogen, alkyl, alkenyl, alkynyl, cycloalkyl, cycloalkenyl, heterocyclyl, aryl, 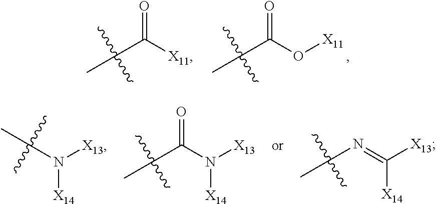
wherein, the “alkyl” “alkenyl” or “alkynyl” are each independently unsubstituted or substituted by at least one group selected from halogen, cyano, nitro, cycloalkyl, trialkylsilyl, cycloalkenyl, heterocyclyl, aryl, 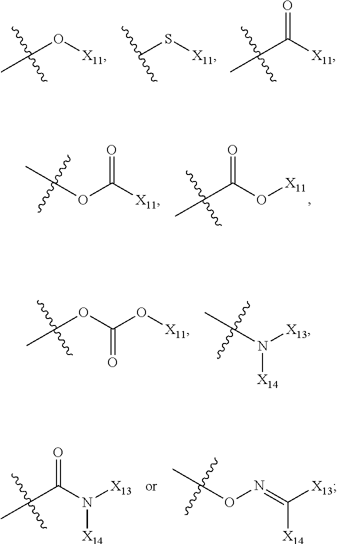
the “cycloalkyl”, “cycloalkenyl”, “heterocyclyl” or “aryl” are each independently unsubstituted or substituted by at least one group selected from oxo, halogen, cyano, nitro, alkyl, alkenyl, alkynyl, cycloalkyl, haloalkyl, haloalkenyl, haloalkynyl, halocycloalkyl, cycloalkyl substituted by alkyl, —OR₁, —SR₁, —(CO)OR₁, —(SO₂)R₁, —N(R₁)₂ or —O-alkyl-(CO)OR₁, or two adjacent carbon atoms on the ring form a fused ring with —OCH₂CH₂— or —OCH₂O— that is unsubstituted or substituted by halogen;
R represents hydrogen, alkyl, alkenyl, alkynyl, cycloalkyl, cycloalkenyl, heterocyclyl, aryl, 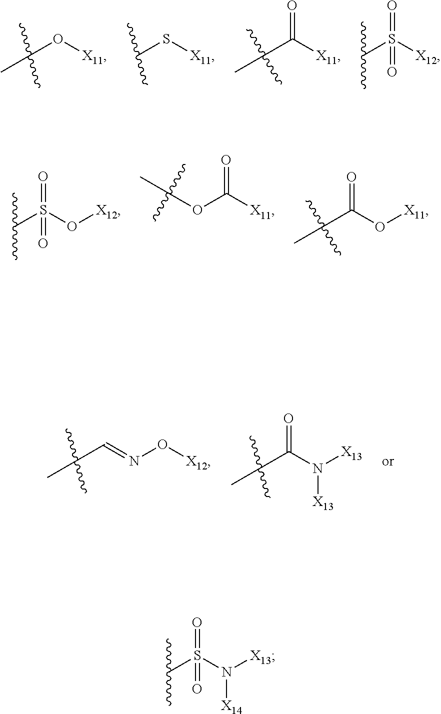
wherein, the “alkyl”, “alkenyl” or “alkynyl” are each independently unsubstituted or substituted by at least one group selected from halogen, cyano, nitro, cycloalkyl, trialkylsilyl, cycloalkenyl, heterocyclyl, aryl, 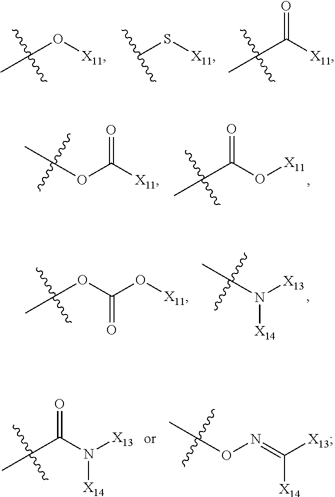
the “cycloalkyl”, “cycloalkenyl”, “heterocyclyl” or “aryl” are each independently unsubstituted or substituted by at least one group selected from oxo, halogen, cyano, nitro, alkyl, alkenyl, alkynyl, cycloalkyl, haloalkyl, haloalkenyl, haloalkynyl, halocycloalkyl, cycloalkyl substituted by alkyl, —OR₁, —SR₁, —(CO)OR₁, —(SO₂)R₁, —N(R₁)₂ or —O-alkyl-(CO)OR₁, or two adjacent carbon atoms on the ring form a fused ring with —OCH₂CH₂— or —OCH₂O— that is unsubstituted or substituted by halogen;
X₁₁ each independently represents hydrogen, alkyl, alkenyl, alkynyl, cycloalkyl, cycloalkylalkyl, cycloalkenyl, cycloalkenylalkyl, heterocyclyl, heterocyclylalkyl, aryl or arylalkyl; wherein, the “alkyl”, “alkenyl” or “alkynyl” are each independently unsubstituted or substituted by at least one group of halogen, —OR₁, —SR₁, —(CO)OR₁, —(SO₂)R₁ or —N(R₁)₂; the “cycloalkyl”, “cycloalkylalkyl”, “cycloalkenyl”, “cycloalkenylalkyl”, “heterocyclyl”, “heterocyclylalkyl”, “aryl” or “arylalkyl” are each independently unsubstituted or substituted by at least one group selected from oxo, halogen, cyano, nitro, alkyl, alkenyl, alkynyl, cycloalkyl, haloalkyl, haloalkenyl, haloalkynyl, halocycloalkyl, cycloalkyl substituted by alkyl, —OR₁, —SR₁, —(CO)OR₁, —(SO₂)R₁, —N(R₁)₂ or —O-alkyl-(CO)OR₁, or two adjacent carbon atoms on the ring form a fused ring with —OCH₂CH₂— or —OCH₂O— that is unsubstituted or substituted by halogen;
X₁₂ each independently represents alkyl, alkenyl, alkynyl, cycloalkyl, cycloalkylalkyl, cycloalkenyl, cycloalkenylalkyl, heterocyclyl, heterocyclylalkyl, aryl or arylalkyl; wherein, the “alkyl”, “alkenyl” or “alkynyl” are each independently unsubstituted or substituted by at least one group of halogen, —OR₁, —SR₁, —(CO)OR₁, —(SO₂)R₁ or —N(R₁)₂; the “cycloalkyl”, “cycloalkylalkyl”, “cycloalkenyl”, “cycloalkenylalkyl”, “heterocyclyl”, “heterocyclylalkyl”, “aryl” or “arylalkyl” are each independently unsubstituted or substituted by at least one group selected from oxo, halogen, cyano, nitro, alkyl, alkenyl, alkynyl, cycloalkyl, haloalkyl, haloalkenyl, haloalkynyl, halocycloalkyl, cycloalkyl substituted by alkyl, —OR₁, —SR₁, —(CO)OR₁, —(SO₂)R₁, —N(R₁)₂ or —O-alkyl-(CO)OR₁, or two adjacent carbon atoms on the ring form a fused ring with —OCH₂CH₂— or —OCH₂O— that is unsubstituted or substituted by halogen;
X₁₃ and X₁₄ each independently represent hydrogen, halogen, cyano, alkoxy, alkoxyalkyl, alkylcarbonyl, alkoxycarbonyl, alkylsulfonyl, alkyl, alkenyl, alkynyl, cycloalkyl, cycloalkylalkyl, cycloalkenyl, cycloalkenylalkyl, aryl, arylalkyl, heterocyclyl or heterocyclylalkyl, or the CX₁₃X₁₄ group together forms an unsubstituted or substituted ring structure, or the NX₁₃X₁₄ group together forms an unsubstituted or substituted heterocyclyl with nitrogen atom at 1-position; wherein, the “alkyl”, “alkenyl” or “alkynyl” are each independently unsubstituted or substituted by at least one group of halogen, —OR₁, —SR₁, —(CO)OR₁, —(SO₂)R₁ or —N(R₁)₂; the “cycloalkyl”, “cycloalkylalkyl”, “cycloalkenyl”, “cycloalkenylalkyl”, “aryl”, “arylalkyl”, “heterocyclyl” or “heterocyclylalkyl” are each independently unsubstituted or substituted by at least one group selected from oxo, halogen, cyano, nitro, alkyl, alkenyl, alkynyl, cycloalkyl, haloalkyl, haloalkenyl, haloalkynyl, halocycloalkyl, cycloalkyl substituted by alkyl, —OR₁, —SR₁, —(CO)OR₁, —(SO₂)R₁, —N(R₁)₂ or —O-alkyl-(CO)OR₁, or two adjacent carbon atoms on the ring form a fused ring with —OCH₂CH₂— or —OCH₂O— that is unsubstituted or substituted by halogen;
R₁ each independently represents hydrogen, alkyl, haloalkyl, phenyl or phenyl substituted by at least one group selected from halogen, cyano, nitro, alkyl, haloalkyl, alkoxycarbonyl, alkylthio, alkylsulfonyl, alkoxy or haloalkoxy.

2. The isoxazoline-substituted benzamide compound, the stereoisomer and the agriculturally or veterinarily acceptable salt thereof according to claim 1, wherein,
X represents hydrogen, halogen, C1-C8 alkoxy or C1-C8 alkylthio;
Y represents halogen or halo C1-C8 alkyl;
Z and Z₁ each independently represent hydrogen, halogen, C1-C8 alkyl, C2-C8 alkenyl, C2-C8 alkynyl, C3-C8 cycloalkyl, halo C1-C8 alkyl or cyano;
Q and Q₁ each independently represent hydrogen, halogen, hydroxyl, cyano, amino, nitro, formyl, cyano C1-C8 alkyl, hydroxyl C1-C8 alkyl, C1-C8 alkyl, C2-C8 alkenyl, C2-C8 alkynyl, C3-C8 cycloalkyl, C3-C8 cycloalkyl C1-C8 alkyl, C1-C8 alkoxy, C1-C8 alkylthio, C1-C8 alkylamino, halo C1-C8 alkoxy, halo C1-C8 alkylthio, C1-C8 alkylcarbonyl, C1-C8 alkoxycarbonyl, C1-C8 alkoxy C1-C8 alkyl, halo C1-C8 alkoxy C1-C8 alkyl, C1-C8 alkylamino C1-C8 alkyl, aryl, heterocyclyl, aryl C1-C8 alkyl or heterocyclyl C1-C8 alkyl; wherein, the “C1-C8 alkyl”, “C2-C8 alkenyl” or “C2-C8 alkynyl” are each independently unsubstituted or substituted by at least one group of halogen, —OR₁, —SR₁, —(CO)OR₁, —(CO)N(R₁)₂, —(SO₂)R₁ or —N(R₁)₂; the “C3-C8 cycloalkyl”, “C3-C8 cycloalkyl C1-C8 alkyl”, “aryl”, “heterocyclyl”, “aryl C1-C8 alkyl” or “heterocyclyl C1-C8 alkyl” are each independently unsubstituted or substituted by at least one group selected from oxo, halogen, cyano, nitro, C1-C8 alkyl, C2-C8 alkenyl, C2-C8 alkynyl, C3-C8 cycloalkyl, halo C1-C8 alkyl, halo C2-C8 alkenyl, halo C2-C8 alkynyl, halo C3-C8 cycloalkyl, C3-C8 cycloalkyl substituted by C1-C8 alkyl, —OR₁, —SR₁, —(CO)OR₁, —(SO₂)R₁, —N(R₁)₂ or —O—(C1-C8 alkyl)-(CO)OR₁, or two adjacent carbon atoms on the ring form a fused ring with —OCH₂CH₂— or —OCH₂O— that is unsubstituted or substituted by halogen;
M represents hydrogen, C1-C8 alkyl, C2-C8 alkenyl, C2-C8 alkynyl, C3-C8 cycloalkyl, C3-C8 cycloalkenyl, heterocyclyl, aryl, 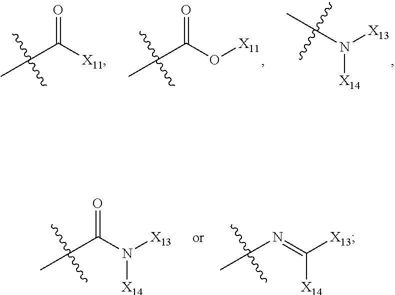
wherein, the “C1-C8 alkyl”, “C2-C8 alkenyl” or “C2-C8 alkynyl” are each independently unsubstituted or substituted by at least one group selected from halogen, cyano, nitro, C3-C8 cycloalkyl, tri(C1-C8)alkylsilyl, C3-C8 cycloalkenyl, heterocyclyl, aryl, 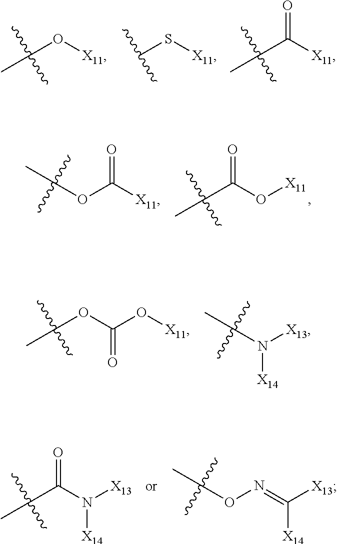
the “C3-C8 cycloalkyl”, “C3-C8 cycloalkenyl”, “heterocyclyl” or “aryl” are each independently unsubstituted or substituted by at least one group selected from oxo, halogen, cyano, nitro, C1-C8 alkyl, C2-C8 alkenyl, C2-C8 alkynyl, C3-C8 cycloalkyl, halo C1-C8 alkyl, halo C2-C8 alkenyl, halo C2-C8 alkynyl, halo C3-C8 cycloalkyl, C3-C8 cycloalkyl substituted by C1-C8 alkyl, —OR₁, —SR₁, —(CO)OR₁, —(SO₂)R₁, —N(R₁)₂ or —O—(C1-C8 alkyl)-(CO)OR₁, or two adjacent carbon atoms on the ring form a fused ring with —OCH₂CH₂— or —OCH₂O— that is unsubstituted or substituted by halogen;
R represents hydrogen, C1-C8 alkyl, C2-C8 alkenyl, C2-C8 alkynyl, C3-C8 cycloalkyl, C3-C8 cycloalkenyl, heterocyclyl, aryl, 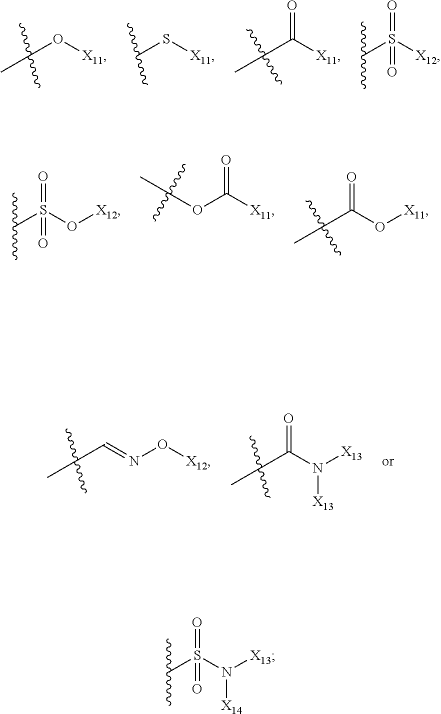
wherein, the “C1-C8 alkyl”, “C2-C8 alkenyl” or “C2-C8 alkynyl” are each independently unsubstituted or substituted by at least one group selected from halogen, cyano, nitro, C3-C8 cycloalkyl, tri(C1-C8)alkylsilyl, C3-C8 cycloalkenyl, heterocyclyl, aryl, 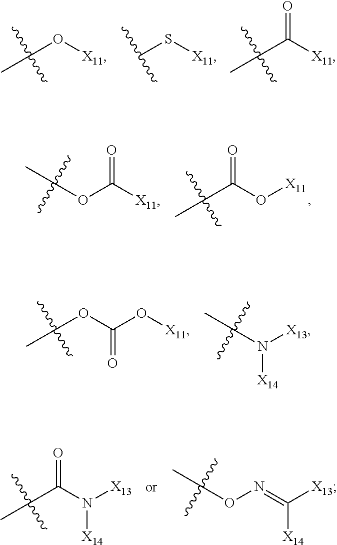
the “C3-C8 cycloalkyl”, “C3-C8 cycloalkenyl”, “heterocyclyl” or “aryl” are each independently unsubstituted or substituted by at least one group selected from oxo, halogen, cyano, nitro, C1-C8 alkyl, C2-C8 alkenyl, C2-C8 alkynyl, C3-C8 cycloalkyl, halo C1-C8 alkyl, halo C2-C8 alkenyl, halo C2-C8 alkynyl, halo C3-C8 cycloalkyl, C3-C8 cycloalkyl substituted by C1-C8 alkyl, —OR₁, —SR₁, —(CO)OR₁, —(SO₂)R₁, —N(R₁)₂ or —O—(C1-C8 alkyl)-(CO)OR₁, or two adjacent carbon atoms on the ring form a fused ring with —OCH₂CH₂— or —OCH₂O— that is unsubstituted or substituted by halogen;
X₁₁ each represents hydrogen, C1-C8 alkyl, C2-C8 alkenyl, C2-C8 alkynyl, C3-C8 cycloalkyl, C3-C8 cycloalkyl C1-C8 alkyl, C3-C8 cycloalkenyl, C3-C8 cycloalkenyl C1-C8 alkyl, heterocyclyl, heterocyclyl C1-C8 alkyl, aryl or aryl C1-C8 alkyl; wherein, the “C1-C8 alkyl”, “C2-C8 alkenyl” or “C2-C8 alkynyl” are each independently unsubstituted or substituted by at least one group of halogen, —OR₁, —SR₁, —(CO)OR₁, —(SO₂)R₁ or —N(R₁)₂;
the “C3-C8 cycloalkyl”, “C3-C8 cycloalkyl C1-C8 alkyl”, “C3-C8 cycloalkenyl”, “C3-C8 cycloalkenyl C1-C8 alkyl”, “heterocyclyl”, “heterocyclyl C1-C8 alkyl”, “aryl” or “aryl C1-C8 alkyl” are each independently unsubstituted or substituted by at least one group selected from oxo, halogen, cyano, nitro, C1-C8 alkyl, C2-C8 alkenyl, C2-C8 alkynyl, C3-C8 cycloalkyl, halo C1-C8 alkyl, halo C2-C8 alkenyl, halo C2-C8 alkynyl, halo C3-C8 cycloalkyl, C3-C8 cycloalkyl substituted by C1-C8 alkyl, —OR₁, —SR₁, —(CO)OR₁, —(SO₂)R₁, —N(R₁)₂ or —O—(C1-C8 alkyl)-(CO)OR₁, or two adjacent carbon atoms on the ring form a fused ring with —OCH₂CH₂— or —OCH₂O— that is unsubstituted or substituted by halogen;
X₁₂ each independently represents C1-C8 alkyl, C2-C8 alkenyl, C2-C8 alkynyl, C3-C8 cycloalkyl, C3-C8 cycloalkyl C1-C8 alkyl, C3-C8 cycloalkenyl, C3-C8 cycloalkenyl C1-C8 alkyl, heterocyclyl, heterocyclyl C1-C8 alkyl, aryl or aryl C1-C8 alkyl; wherein, the “C1-C8 alkyl”, “C2-C8 alkenyl” or “C2-C8 alkynyl” are each independently unsubstituted or substituted by at least one group of halogen, —OR₁, —SR₁, —(CO)OR₁, —(SO₂)R₁ or —N(R₁)₂; the “C3-C8 cycloalkyl”, “C3-C8 cycloalkyl C1-C8 alkyl”, “C3-C8 cycloalkenyl”, “C3-C8 cycloalkenyl C1-C8 alkyl”, “heterocyclyl”, “heterocyclyl C1-C8 alkyl”, “aryl” or “aryl C1-C8 alkyl” are each independently unsubstituted or substituted by at least one group selected from oxo, halogen, cyano, nitro, C1-C8 alkyl, C2-C8 alkenyl, C2-C8 alkynyl, C3-C8 cycloalkyl, halo C1-C8 alkyl, halo C2-C8 alkenyl, halo C2-C8 alkynyl, halo C3-C8 cycloalkyl, C3-C8 cycloalkyl substituted by C1-C8 alkyl, —OR₁, —SR₁, —(CO)OR₁, —(SO₂)R₁, —N(R₁)₂ or —O—(C1-C8 alkyl)-(CO)OR₁, or two adjacent carbon atoms on the ring form a fused ring with —OCH₂CH₂— or —OCH₂O— that is unsubstituted or substituted by halogen;
X₁₃ and X₁₄ each independently represent hydrogen, halogen, cyano, C1-C8 alkoxy, C1-C8 alkoxy C1-C8 alkyl, C1-C8 alkylcarbonyl, C1-C8 alkoxycarbonyl, C1-C8 alkylsulfonyl, C1-C8 alkyl, C2-C8 alkenyl, C2-C8 alkynyl, C3-C8 cycloalkyl, C3-C8 cycloalkyl C1-C8 alkyl, C3-C8 cycloalkenyl, C3-C8 cycloalkenyl C1-C8 alkyl, aryl, aryl C1-C8 alkyl, heterocyclyl or heterocyclyl C1-C8 alkyl, or the CX₁₃X₁₄ group together forms a 5-8 membered carbocycle or oxygen-, sulfur- or nitrogen-containing heterocycle, or the NX₁₃X₁₄ group together forms 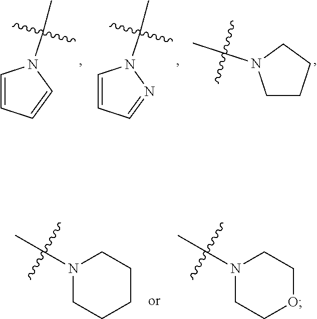
wherein, the “C1-C8 alkyl”, “C2-C8 alkenyl” or “C2-C8 alkynyl” are each independently unsubstituted or substituted by at least one group of halogen, —OR₁, —SR₁, —(CO)OR₁, —(SO₂)R₁ or —N(R₁)₂; the “C3-C8 cycloalkyl”, “C3-C8 cycloalkyl C1-C8 alkyl”, “C3-C8 cycloalkenyl”, “C3-C8 cycloalkenyl C1-C8 alkyl”, “aryl”, “aryl C1-C8 alkyl”, “heterocyclyl” or “heterocyclyl C1-C8 alkyl” are each independently unsubstituted or substituted by at least one group selected from oxo, halogen, cyano, nitro, C1-C8 alkyl, C2-C8 alkenyl, C2-C8 alkynyl, C3-C8 cycloalkyl, halo C1-C8 alkyl, halo C2-C8 alkenyl, halo C2-C8 alkynyl, halo C3-C8 cycloalkyl, C3-C8 cycloalkyl substituted by C1-C8 alkyl, —OR₁, —SR₁, —(CO)OR₁, —(SO₂)R₁, —N(R₁)₂ or —O—(C1-C8 alkyl)-(CO)OR₁, or two adjacent carbon atoms on the ring form a fused ring with —OCH₂CH₂— or —OCH₂O— that is unsubstituted or substituted by halogen; the “5-8 membered carbocycle or oxygen-, sulfur- or nitrogen-containing heterocycle” is unsubstituted or substituted by at least one group selected from C1-C8 alkyl, C1-C8 alkoxycarbonyl or benzyl, or forms a fused ring structure with aryl or heterocyclyl; the 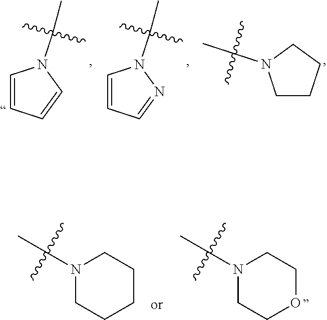 [SMILES: *n1cccc1 ; *n1cccn1 ; *N1CCCC1 ; *N1CCCCC1 ; *N1CCOCC1]
is unsubstituted or substituted by at least one group selected from oxo, C1-C8 alkyl or C1-C8 alkoxycarbonyl;
R₁ each independently represents hydrogen, C1-C8 alkyl, halo C1-C8 alkyl, phenyl or phenyl substituted by at least one group selected from halogen, cyano, nitro, C1-C8 alkyl, halo C1-C8 alkyl, C1-C8 alkoxycarbonyl, C1-C8 alkylthio, C1-C8 alkylsulfonyl, C1-C8 alkoxy or halo C1-C8 alkoxy.

3. The isoxazoline-substituted benzamide compound, the stereoisomer and the agriculturally or veterinarily acceptable salt thereof according to claim 1, wherein
X represents hydrogen, halogen, C1-C6 alkoxy or C1-C6 alkylthio;
Y represents halogen or halo C1-C6 alkyl;
Z and Z₁ each independently represent hydrogen, halogen, C1-C6 alkyl, C2-C6 alkenyl, C2-C6 alkynyl, C3-C6 cycloalkyl, halo C1-C6 alkyl or cyano;
Q and Q₁ each independently represent hydrogen, halogen, hydroxyl, cyano, amino, nitro, formyl, cyano C1-C6 alkyl, hydroxyl C1-C6 alkyl, C1-C6 alkyl, C2-C6 alkenyl, C2-C6 alkynyl, C3-C6 cycloalkyl, C3-C6 cycloalkyl C1-C6 alkyl, C1-C6 alkoxy, C1-C6 alkylthio, C1-C6 alkylamino, halo C1-C6 alkoxy, halo C1-C6 alkylthio, C1-C6 alkylcarbonyl, C1-C6 alkoxycarbonyl, C1-C6 alkoxy C1-C6 alkyl, halo C1-C6 alkoxy C1-C6 alkyl, C1-C6 alkylamino C1-C6 alkyl, aryl, heterocyclyl, aryl C1-C6 alkyl or heterocyclyl C1-C6 alkyl; wherein, the “C1-C6 alkyl”, “C2-C6 alkenyl” or “C2-C6 alkynyl” are each independently unsubstituted or substituted by at least one group of halogen, —OR₁, —SR₁, —(CO)OR₁, —(CO)N(R₁)₂, —(SO₂)R₁ or —N(R₁)₂; the “C3-C6 cycloalkyl”, “C3-C6 cycloalkyl C1-C6 alkyl”, “aryl”, “heterocyclyl”, “aryl C1-C6 alkyl” or “heterocyclyl C1-C6 alkyl” are each independently unsubstituted or substituted by at least one group selected from oxo, halogen, cyano, nitro, C1-C6 alkyl, C2-C6 alkenyl, C2-C6 alkynyl, C3-C6 cycloalkyl, halo C1-C6 alkyl, halo C2-C6 alkenyl, halo C2-C6 alkynyl, halo C3-C6 cycloalkyl, C3-C6 cycloalkyl substituted by C1-C6 alkyl, —OR₁, —SR₁, —(CO)OR₁, —(SO₂)R₁, —N(R₁)₂ or —O—(C1-C6 alkyl)-(CO)OR₁, or two adjacent carbon atoms on the ring form a fused ring with —OCH₂CH₂— or —OCH₂O— that is unsubstituted or substituted by halogen;
M represents hydrogen, C1-C6 alkyl, C2-C6 alkenyl, C2-C6 alkynyl, C3-C6 cycloalkyl, C3-C6 cycloalkenyl, heterocyclyl, aryl, 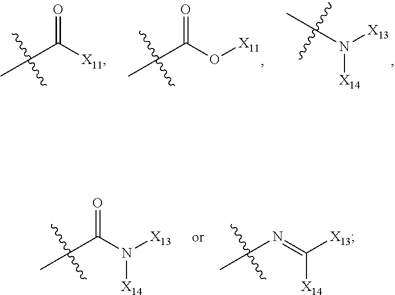
wherein, the “C1-C6 alkyl”, “C2-C6 alkenyl” or “C2-C6 alkynyl” are each independently unsubstituted or substituted by at least one group selected from halogen, cyano, nitro, C3-C6 cycloalkyl, tri(C1-C6)alkylsilyl, C3-C6 cycloalkenyl, heterocyclyl, aryl, 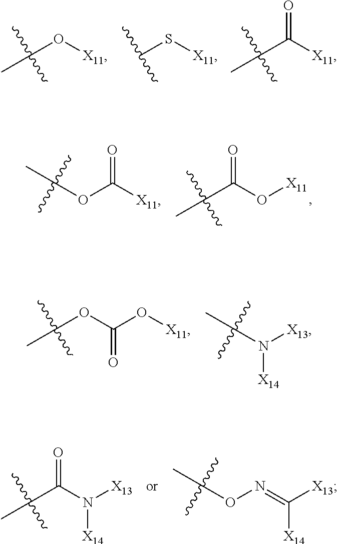
the “C3-C6 cycloalkyl”, “C3-C6 cycloalkenyl”, “heterocyclyl” or “aryl” are each independently unsubstituted or substituted by at least one group selected from oxo, halogen, cyano, nitro, C1-C6 alkyl, C2-C6 alkenyl, C2-C6 alkynyl, C3-C6 cycloalkyl, halo C1-C6 alkyl, halo C2-C6 alkenyl, halo C2-C6 alkynyl, halo C3-C6 cycloalkyl, C3-C6 cycloalkyl substituted by C1-C6 alkyl, —OR₁, —SR₁, —(CO)OR₁, —(SO₂)R₁, —N(R₁)₂ or —O—(C1-C6 alkyl)-(CO)OR₁, or two adjacent carbon atoms on the ring form a fused ring with —OCH₂CH₂— or —OCH₂O— that is unsubstituted or substituted by halogen;
R represents hydrogen, C1-C6 alkyl, C2-C6 alkenyl, C2-C6 alkynyl, C3-C6 cycloalkyl, C3-C6 cycloalkenyl, heterocyclyl, aryl, 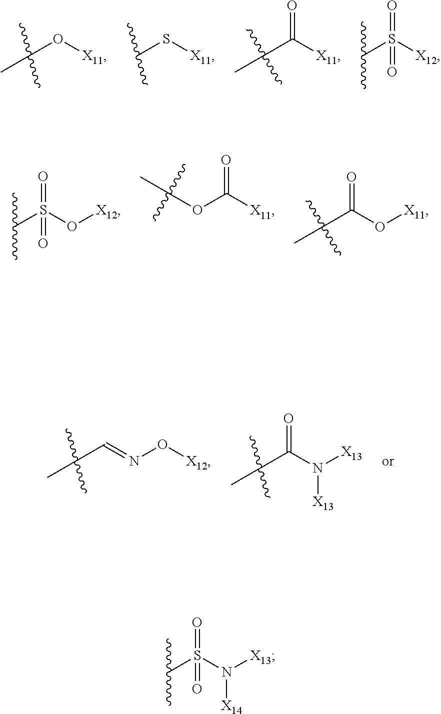
wherein, the “C1-C6 alkyl”, “C2-C6 alkenyl” or “C2-C6 alkynyl” are each independently unsubstituted or substituted by at least one group selected from halogen, cyano, nitro, C3-C6 cycloalkyl, tri(C1-C6)alkylsilyl, C3-C6 cycloalkenyl, heterocyclyl, aryl, 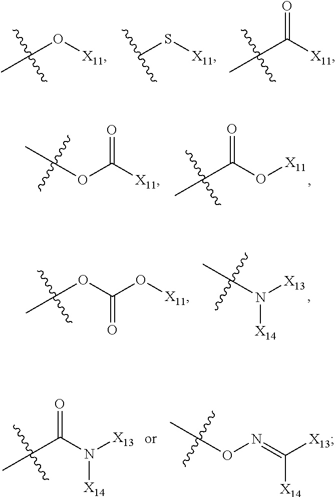
the “C3-C6 cycloalkyl”, “C3-C6 cycloalkenyl”, “heterocyclyl” or “aryl” are each independently unsubstituted or substituted by at least one group selected from oxo, halogen, cyano, nitro, C1-C6 alkyl, C2-C6 alkenyl, C2-C6 alkynyl, C3-C6 cycloalkyl, halo C1-C6 alkyl, halo C2-C6 alkenyl, halo C2-C6 alkynyl, halo C3-C6 cycloalkyl, C3-C6 cycloalkyl substituted by C1-C6 alkyl, —OR₁, —SR₁, —(CO)OR₁, —(SO₂)R₁, —N(R₁)₂ or —O—(C1-C6 alkyl)-(CO)OR₁, or two adjacent carbon atoms on the ring form a fused ring with —OCH₂CH₂— or —OCH₂O— that is unsubstituted or substituted by halogen;
X₁₁ each independently represents hydrogen, C1-C6 alkyl, C2-C6 alkenyl, C2-C6 alkynyl, C3-C6 cycloalkyl, C3-C6 cycloalkyl C1-C6 alkyl, C3-C6 cycloalkenyl, C3-C6 cycloalkenyl C1-C6 alkyl, heterocyclyl, heterocyclyl C1-C6 alkyl, aryl or aryl C1-C6 alkyl; wherein, the “C1-C6 alkyl”, “C2-C6 alkenyl” or “C2-C6 alkynyl” are each independently unsubstituted or substituted by at least one group of halogen, —OR₁, —SR₁, —(CO)OR₁, —(SO₂)R₁ or —N(R₁)₂; the “C3-C6 cycloalkyl”, “C3-C6 cycloalkyl C1-C6 alkyl”, “C3-C6 cycloalkenyl”, “C3-C6 cycloalkenyl C1-C6 alkyl”, “heterocyclyl”, “heterocyclyl C1-C6 alkyl”, “aryl” or “aryl C1-C6 alkyl” are each independently unsubstituted or substituted by at least one group selected from oxo, halogen, cyano, nitro, C1-C6 alkyl, C2-C6 alkenyl, C2-C6 alkynyl, C3-C6 cycloalkyl, halo C1-C6 alkyl, halo C2-C6 alkenyl, halo C2-C6 alkynyl, halo C3-C6 cycloalkyl, C3-C6 cycloalkyl substituted by C1-C6 alkyl, —OR₁, —SR₁, —(CO)OR₁, —(SO₂)R₁, —N(R₁)₂ or —O—(C1-C6 alkyl)-(CO)OR₁, or two adjacent carbon atoms on the ring form a fused ring with —OCH₂CH₂ or —OCH₂O— that is unsubstituted or substituted by halogen;
X₁₂ each independently represents C1-C6 alkyl, C2-C6 alkenyl, C2-C6 alkynyl, C3-C6 cycloalkyl, C3-C6 cycloalkyl C1-C6 alkyl, C3-C6 cycloalkenyl, C3-C6 cycloalkenyl C1-C6 alkyl, heterocyclyl, heterocyclyl C1-C6 alkyl, aryl or aryl C1-C6 alkyl; wherein, the “C1-C6 alkyl”, “C2-C6 alkenyl” or “C2-C6 alkynyl” are each independently unsubstituted or substituted by at least one group of halogen, —OR₁, —SR₁, —(CO)OR₁, —(SO₂)R₁ or —N(R₁)₂; the “C3-C6 cycloalkyl”, “C3-C6 cycloalkyl C1-C6 alkyl”, “C3-C6 cycloalkenyl”, “C3-C6 cycloalkenyl C1-C6 alkyl”, “heterocyclyl”, “heterocyclyl C1-C6 alkyl”, “aryl” or “aryl C1-C6 alkyl” are each independently unsubstituted or substituted by at least one group selected from oxo, halogen, cyano, nitro, C1-C6 alkyl, C2-C6 alkenyl, C2-C6 alkynyl, C3-C6 cycloalkyl, halo C1-C6 alkyl, halo C2-C6 alkenyl, halo C2-C6 alkynyl, halo C3-C6 cycloalkyl, C3-C6 cycloalkyl substituted by C1-C6 alkyl, —OR₁, —SR₁, —(CO)OR₁, —(SO₂)R₁, —N(R₁)₂ or —O—(C1-C6 alkyl)-(CO)OR₁, or two adjacent carbon atoms on the ring form a fused ring with —OCH₂CH₂— or —OCH₂O— that is unsubstituted or substituted by halogen;
X₁₃ and X₁₄ each independently represent hydrogen, halogen, cyano, C1-C6 alkoxy, C1-C6 alkoxy C1-C6 alkyl, C1-C6 alkylcarbonyl, C1-C6 alkoxycarbonyl, C1-C6 alkylsulfonyl, C1-C6 alkyl, C2-C6 alkenyl, C2-C6 alkynyl, C3-C6 cycloalkyl, C3-C6 cycloalkyl C1-C6 alkyl, C3-C6 cycloalkenyl, C3-C6 cycloalkenyl C1-C6 alkyl, aryl, aryl C1-C6 alkyl, heterocyclyl or heterocyclyl C1-C6 alkyl, or the CX₁₃X₁₄ group together forms a 5-8 membered carbocycle or oxygen-, sulfur- or nitrogen-containing heterocycle, or the NX₁₃X₁₄ group together forms 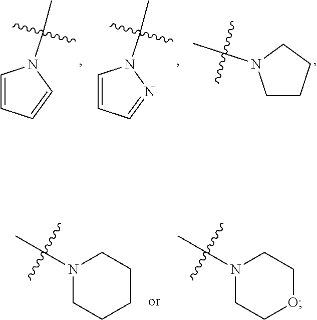
wherein, the “C1-C6 alkyl”, “C2-C6 alkenyl” or “C2-C6 alkynyl” are each independently unsubstituted or substituted by at least one group of halogen, —OR₁, —SR₁, —(CO)OR₁, —(SO₂)R₁ or —N(R₁)₂; the “C3-C6 cycloalkyl”, “C3-C6 cycloalkyl C1-C6 alkyl”, “C3-C6 cycloalkenyl”, “C3-C6 cycloalkenyl C1-C6 alkyl”, “aryl”, “aryl C1-C6 alkyl”, “heterocyclyl” or “heterocyclyl C1-C6 alkyl” are each independently unsubstituted or substituted by at least one group selected from oxo, halogen, cyano, nitro, C1-C6 alkyl, C2-C6 alkenyl, C2-C6 alkynyl, C3-C6 cycloalkyl, halo C1-C6 alkyl, halo C2-C6 alkenyl, halo C2-C6 alkynyl, halo C3-C6 cycloalkyl, C3-C6 cycloalkyl substituted by C1-C6 alkyl, —OR₁, —SR₁, —(CO)OR₁, —(SO₂)R₁, —N(R₁)₂ or —O—(C1-C6 alkyl)-(CO)OR₁, or two adjacent carbon atoms on the ring form a fused ring with —OCH₂CH₂— or —OCH₂O— that is unsubstituted or substituted by halogen; the “5-8 membered carbocycle or oxygen-, sulfur- or nitrogen-containing heterocycle” is unsubstituted or substituted by at least one group selected from C1-C6 alkyl, C1-C6 alkoxycarbonyl or benzyl, or forms a fused ring structure with aryl or heterocyclyl; the 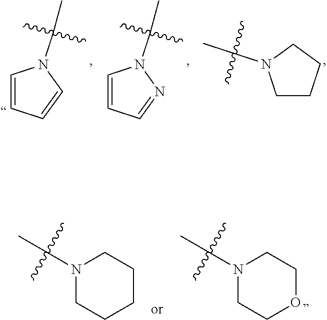 [SMILES: *n1cccc1 ; *n1cccn1 ; *N1CCCC1 ; *N1CCCCC1 ; *N1CCOCC1]
is unsubstituted or substituted by at least one group selected from oxo, C1-C6 alkyl or C1-C6 alkoxycarbonyl;
R₁ each independently represents hydrogen, C1-C6 alkyl, halo C1-C6 alkyl, phenyl or phenyl substituted by at least one group selected from halogen, cyano, nitro, C1-C6 alkyl, halo C1-C6 alkyl, C1-C6 alkoxycarbonyl, C1-C6 alkylthio, C1-C6 alkylsulfonyl, C1-C6 alkoxy or halo C1-C6 alkoxy.

4. An isoxazoline-substituted benzamide compound with a chiral center as shown in Formula I′, a stereoisomer and an agriculturally or veterinarily acceptable salt thereof: 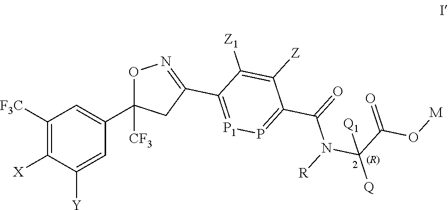
wherein, the definitions of substituent groups X, Y, Z, Z₁, P, P₁, R, Q, Q₁ and M are as shown in claim 1; the carbon atom at position 2 is a chiral center; based on the contents of stereoisomers that have R- and S-configurations at this position, it has a stereochemical purity of 60-100% (R).

5. An isoxazoline-substituted benzamide with a chiral center as shown in Formula I″, a stereoisomer and an agriculturally or veterinarily acceptable salt thereof: 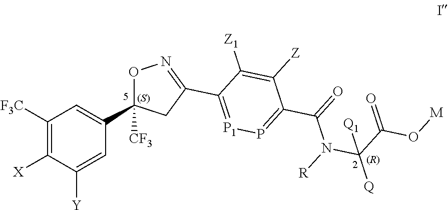
wherein, the definitions of substituent groups X, Y, Z, Z₁, P, P₁, R, Q, Q₁ and M are as shown in claim 1;
based on the contents of stereoisomers that have R- and S-configurations at position 5, it has a stereochemical purity of 60-100% (S);
and the carbon atom at position 2 is a chiral center; based on the contents of stereoisomers that have R- and S-configurations at this position, it has a stereochemical purity of 60-100% (R).

6. The isoxazoline-substituted benzamide compound, the stereoisomer and the agriculturally or veterinarily acceptable salt thereof according to claim 1, wherein they are characterized in that the compound is selected from any one of Table 1, Table A and Table B.

7. A preparation method of the isoxazoline-substituted benzamide compound, the stereoisomer and the agriculturally or veterinary acceptable salt thereof according to claim 1, comprising the following steps:
in the presence of a condensing agent, the compound as shown in Formula I is prepared by reacting the compound shown in Formula II with the compound shown in Formula III, and the chemical reaction equation is as follows: 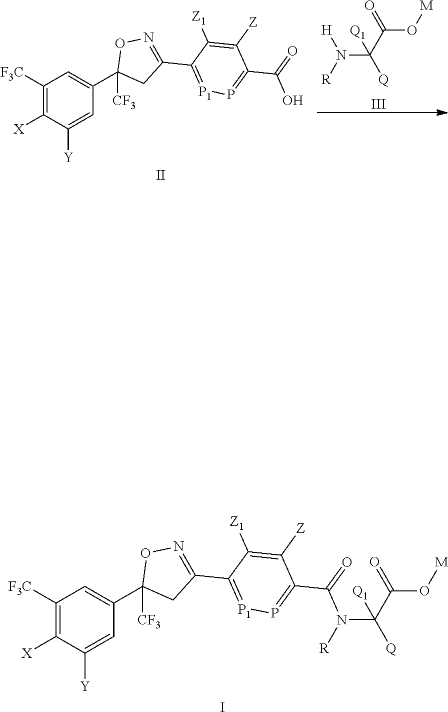
wherein, the definitions of substituent groups X, Y, Z, Z₁, P, P₁, R, Q, Q₁ and M are as described in claim 1; the reaction is carried out in the presence of a base and a solvent.

8. An insecticidal composition, wherein the insecticidal composition comprises a biologically effective dose of at least one of the isoxazoline-substituted benzamide compound, the stereoisomer and the agriculturally or veterinary acceptable salt thereof according to claim 1, and a formulation auxiliary.

9. A method for controlling pest insects, wherein it is characterized in that it comprises exposing a pest insect or its environment to a biologically effective dose of the isoxazoline-substituted benzamide compound, the stereoisomer and the agriculturally or veterinary acceptable salt thereof according to claim 1.

10. (canceled)

11. The isoxazoline-substituted benzamide compound, the stereoisomer and the agriculturally or veterinarily acceptable salt thereof according to claim 1, wherein
X represents hydrogen, fluorine, chlorine, methoxy or methylthio;
Y represents chlorine or fluorine;
Z represents hydrogen, methyl, ethyl, isopropyl, chlorine, trifluoromethyl or vinyl;
Z₁ represents hydrogen or chlorine;
Q represents hydrogen, methyl, isopropyl, cyclopropyl, methoxy, trifluoromethyl, benzyl, 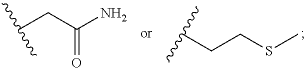
Q₁ represents hydrogen;
P represents CH or N;
P₁ represents CH or N;
M represents hydrogen, methyl, ethyl, allyl, propargyl, 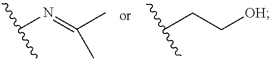
R represents hydrogen or methyl.

12. The isoxazoline-substituted benzamide compound with a chiral center, a stereoisomer and an agriculturally or veterinarily acceptable salt thereof according to claim 4, wherein it has a stereochemical purity of 80-100% (R).

13. The isoxazoline-substituted benzamide compound with a chiral center, a stereoisomer and an agriculturally or veterinarily acceptable salt thereof according to claim 12, wherein it has a stereochemical purity of 90-100% (R).

14. The isoxazoline-substituted benzamide compound with a chiral center, a stereoisomer and an agriculturally or veterinarily acceptable salt thereof according to claim 13, wherein it has a stereochemical purity of 95-100% (R).

15. The isoxazoline-substituted benzamide with a chiral center, a stereoisomer and an agriculturally or veterinarily acceptable salt thereof according to claim 5, wherein based on the contents of stereoisomers that have R- and S-configurations at position 5, it has a stereochemical purity of 80-100% (S),
and the carbon atom at position 2 is a chiral center; based on the contents of stereoisomers that have R- and S-configurations at this position, it has a stereochemical purity of 80-100% (R).

16. The isoxazoline-substituted benzamide with a chiral center, a stereoisomer and an agriculturally or veterinarily acceptable salt thereof according to claim 15, wherein based on the contents of stereoisomers that have R- and S-configurations at position 5, it has a stereochemical purity of 90-100% (S),
and the carbon atom at position 2 is a chiral center; based on the contents of stereoisomers that have R- and S-configurations at this position, it has a stereochemical purity of 90-100% (R).

17. The isoxazoline-substituted benzamide with a chiral center, a stereoisomer and an agriculturally or veterinarily acceptable salt thereof according to claim 16, wherein based on the contents of stereoisomers that have R- and S-configurations at position 5, it has a stereochemical purity of 95-100% (S),
and the carbon atom at position 2 is a chiral center; based on the contents of stereoisomers that have R- and S-configurations at this position, it has a stereochemical purity of 95-100% (R).

18. The preparation method according to claim 7, wherein the condensing agent is PyBOP, HATU, HOBt-EDCI, CDI, DCC or DBU.

19. The insecticidal composition according to claim 8, wherein the another active ingredient is also included, the another active ingredient is chlorantraniliprole, spinetoram, lambda-cyhalothrin, emamectin-benzoate, abamectin, indoxacarb, lufenuron, chlorfenapyr, spirotetramat, triflumezopyrim, diafenthiuron, cyetpyrafen, acephate, bifenthrin, methoxyfenozide, spiromesifen, fipronil, spiropidion, buprofezin, pymetrozine, clothianidin, nitenpyram, acetamiprid, afidopyropen, cyantraniliprole, dinotefuran, ethiprole, imidacloprid, sulfoxaflor, thiamethoxam, tolfenpyrad or dimpropyridaz.

20. A method for controlling pest insects, wherein it is characterized in that it comprises exposing a pest insect or its environment to a biologically effective dose of the composition according to claim 8.
